(19) United States
(12) Patent Application Publication
(10) Pub. No.: US 2022/0000791 A1
(43) Pub. Date: Jan. 6, 2022

(54) MULTI-LAYERED PARTICLES

(71) Applicant: SPHERA ENCAPSULATION SRL, Verona (IT)

(72) Inventors: Francesca ZANONI, Cerro Veronese (IT); Gianni ZOCCATELLI, Bussolengo (IT); Martina VAKARELOVA, Verona (IT); Roberto CHIGNOLA, Lavagno (IT)

(21) Appl. No.: 17/295,722

(22) PCT Filed: Nov. 20, 2019
(86) PCT No.: PCT/IB2019/059991

§ 371 Date: May 20, 2021

Related U.S. Application Data
(60) Provisional application No. 62/769,642, filed on Nov. 20, 2018.

(51) Int. Cl. A61K 9/51; A61K 31/122; A61K 31/12; A61K 31/202; A61K 31/522; A61K 31/015; A61K 35/60; A61K 36/28; A61K 31/575; A61K 31/593; A61K 31/352; A61K 36/185

(52) U.S. Cl. CPC A61K 9/5169; A61K 9/5161; A61K 31/122; A61K 31/12; A61K 31/202; A61K 31/522; A61K 9/5192; A61K 35/60; A61K 36/28; A61K 31/575; A61K 31/593; A61K 31/352; A61K 36/185; A61K 31/015

(57) ABSTRACT

Multi-layered particles and compositions comprising a compound are disclosed. Processes of preparing the particles and compositions and uses thereof are also disclosed.

### CROSS REFERENCE TO RELATED APPLICATIONS

This application claims the benefit of priority to U.S. Provisional Patent Application No. 62/769,642 filed Nov. 20, 2018 entitled “MULTI-LAYERED PARTICLES”, the contents of which are incorporated herein by reference in their entirety.

### FIELD OF THE INVENTION

The present invention is in the field of multi-layered particles, compositions comprising same, processes of preparing such compositions and uses thereof.

### BACKGROUND OF THE INVENTION

The efficacy of many bioactive agents is based on their ability to reach the selected target sites and remain present in effective concentrations for sufficient periods of time to accomplish the desired therapeutic or diagnostic purpose. The molecular properties of a bioactive compound may impair the absorption through a given delivery route, thereby resulting in a substantial reduction in efficacy. Lipophilic substances possessing low water solubility often have poor oral bioavailability. These substances, being hydrophobic by nature, show wetting difficulties and poor dissolution. These properties obviously represent a rate-limiting step in their absorption from solid oral dosage forms and, in turn, cause a subsequent reduction in their bioavailability.

There is an increased need for overcoming the limitations of the conventional encapsulation techniques, and for finding methods of providing solid or liquid, lipophilic active substances and other active ingredients in a stable way, allowing for their controlled release without losing their activity, protection of the same activity when in contact with other components, the masking of unpleasant odor when needed, and masking of taste.

### SUMMARY OF THE INVENTION

According to some embodiments of the present invention, there is provided a particle comprising at least one compound having a protein-based shell at least partially surrounding the at least one compound, and a coating comprising a polysaccharide encapsulating the at least one shelled compound.

In some embodiments, there is provided a particle having a diameter of 5 nm to 10000 nm. In some embodiments, there is provided a particle having a diameter of 1 μm to 500 μm. In some embodiments, at least one compound and a shell have a diameter of 5 nm to 300 nm.

In some embodiments, a protein-based shell at least partially surrounding the compound is surrounding at least 85% of the total surface of at least one compound.

In some embodiments, the particle comprises 1% to 80% (w/w) of the compound.

In some embodiments, the particle comprises 0.1% to 99% (w/w) of the protein-based shell.

In some embodiments, the concentration of a bioactive compound in a particle is 0.01 mg/g to 500 mg/g.

In some embodiments, at least one compound is soluble in an organic solvent.

In some embodiments, at least one compound is selected from the group consisting of a lipophilic compound, volatile organic compound, fragrance, protein, aroma, vitamin, lipophilic metabolite, partially lipophilic metabolite, or any combination thereof.

In some embodiments, the protein-based shell is selected from the group consisting of whey protein, soya protein, pea protein, fava bean protein, collagen or any combination thereof.

In some embodiments, the at least one compound is selected from the group consisting of a astaxanthin, curcumin, omega 3, caffeine, beta-carotene, fish oil, sunflower oil, phytosterol, epigallocatechin gallate, coenzyme Q10, cannabinoid or a functional derivative thereof, vitamin D, or any combination thereof.

In some embodiments, the particle further comprises a cationic polymer interacting with at least a portion of a protein-based shell.

In some embodiments, the interaction is an electrostatic interaction.

In some embodiments, the particle has a diameter of 50 nm to 300 nm.

According to some embodiments of the present invention, there is provided a composition comprising a plurality of particles as disclosed herein.

In some embodiments, the composition is selected from the group consisting of an edible composition, a dietary supplement composition, a pharmaceutical composition, an agrochemical composition, and a cosmetic composition.

In some embodiments, the composition is in the form of a powder. In some embodiments, the powder comprises 1% to 80% (w/w) of the at least one compound.

In some embodiments, the powder has a content of 0.5 mg/g to 500 mg/g of at least a compound.

In some embodiments, at least 80% of the particles have a size in the rage of 5 nm to 300 nm when re-dispersed in water.

In some embodiments, the composition has a polydispersity index of 0.05 to 0.7.

In some embodiments, the at least one compound having a protein-based shell at least partially surrounding the at least one compound has a zeta potential of −50 mV to −10 mV.

In some embodiments, the composition has an antioxidant activity.

In some embodiments, at least 80% of the particles have a size of 50 nm to 300 nm.

In some embodiments, the composition having a polydispersity index of 0.05 to 0.7.

In some embodiments, the particles have a zeta potential of 0 mV to 100 mV.

In some embodiments, 20% to 90% of the compound is released in the intestinal phase under physiological conditions.

According to some embodiments of the present invention, there is provided a method for encapsulating a compound. In some embodiments, there is provided a method comprising the steps of a. mixing a compound and a solvent, b. mixing the compound and the solvent with a protein, or a polysaccharide, or both, thereby obtaining a nano-emulsion, c. evaporating the solvent, thereby obtaining a particle, and d. drying the particle with a protein, a polysaccharide, or a mixture thereof, thereby encapsulating the compound.

In some embodiments, the particle has a diameter of 5 nm to 300 nm.

In some embodiments, the method comprises the step of adding a cationic polymer prior to the drying.

In some embodiments, drying is spray drying, granulating, agglomerating, or any combination thereof, the particles.

In some embodiments, a compound is in a suspension.

In some embodiments, the solvent has a boiling point in the range of 35° C. to 80° C. In some embodiments, the solvent comprises ethyl acetate.

In some embodiments, the compound and the protein are in a ratio of 4:1 to 1:50 (w/w).

In some embodiments, the method has an encapsulation yield of 60% to 95%.

In some embodiments, the method has an encapsulation efficacy of 80% to 100%.

In some embodiments, the concentration of a compound in a particle is 0.01 mg/g to 500 mg/g.

According to some embodiments of the present invention, there is provided a particle produced by a method as described elsewhere herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a bar graph with astaxanthin nanoparticles (NPs ASX) dimension without chitosan (NPs ASX WPI), and with chitosan at two different concentrations;

FIG. 2 is a graph of the zeta potential distribution of the NPs ASX with whey protein isolate (WPI) and NPs ASX WPI after the addition of chitosan;

FIG. 3 is a graph of the size distribution of NPs ASX powder formulation, obtained with whey protein isolate (WPI) and maltodextrin (MD), upon re-dispersion in water;

FIG. 4 shows a HPLC analysis of ASX extract from the powder containing ASX particles before and after spray dry;

FIGS. 5A-D show powder particles morphology evaluation by stereomicroscope image 20× (A), DLS analysis of particles diameters upon dispersion in water (B), and SEM micrograph (C and D);

FIGS. 6A-B are graphs showing the ASX release from whey protein concentrate (WPC) ASX NPs and from powder containing ASX particles during in vitro simulated digestion;

FIG. 7 is a bar graph of the relative concentration of the different ester forms of ASX (free form, monoester and diester) before digestion and after 2 hours of intestinal digestion of the NPs ASX form;

FIG. 8 is a bar graph of the relative concentration of the different ester forms of ASX (free form, monoester and diester) in the powder containing ASX particles before and after digestion;

FIGS. 9A-C are confocal microscopy images showing the cell uptake of the ASX NPs; Caco2 cell line incubated with NPs preparation (average size: 107 nm) (FIG. 9A), HepG2 cell line incubated with NPs preparation (average size: 107 nm (FIG. 9B), J774A1 cell line incubated with resuspended powder containing ASX particles (average size: 220 nm) (FIG. 9C);

FIG. 10 is a graph showing particle size distribution upon resuspension in water of the powder containing ASX particles in comparison to the agglomerates containing ASX particles obtained by fluid bed;

FIG. 11 is a graph showing particle size distribution of curcumin WPC NPs;

FIG. 12 is a graph showing particle size distribution of fish oil WPC NPs;

FIGS. 13A-F are graphs showing the variation of Z-average and PDI (FIG. 13A and FIG. 13C) and zeta-potential (FIG. 13B and FIG. 13D) as a function of protein concentration (FIG. 13A and FIG. 13B) and the H.p. oleoresin concentration (FIG. 13C and FIG. 13D) used to produce NPs. Statistically significant differences (P<0.05) between values are indicated by different letters. In panels A and C only the significance of the values relative to Z-average are indicated, since no differences for PDI were observed (P>0.05). Appearance of the nanoparticles produced as function of protein concentration (FIG. 13E) and H.p. oleoresin concentration (FIG. 13F);

FIGS. 14A-C are graphs showing DLS analysis of the NPs obtained with 1% WPC and 4.5% of H.p. oleoresin; the percentage distributions are reported by number (FIG. 14A), intensity (FIG. 14B) and volume (FIG. 14C). Particles with diameter around 1400 nm in B and C are probably due to the presence of dust and not dependent on the encapsulation process;

FIG. 15 is a graph showing the comparison between the absorption spectra of the H.p. oleoresin and NPs;

FIGS. 16A-B are HPLC chromatograms of H.p. oleoresin (FIG. 16A) and NPs extract composition (FIG. 16B) showing free, mono-esters and di-esters of ASX before and after encapsulation;

FIG. 17 is a bar graph showing the stability of the NPs at different pH values expressed as turbidity measured spectrophotometrically at 660 nm;

FIG. 18 is a graph showing the comparison between ASX retained in NPs and in H.p. oleoresin after exposure to UV rays; the values are given as mean values±standard deviation;

FIG. 19 is a graph showing the comparison between ASX retained in NPs and H.p. oleoresin after exposure to FeCl₃; the values are given as mean values±standard deviation;

FIG. 20 is a graph showing the degradation kinetics of NPs and H.p. oleoresin at 65° C.; the values are given as mean values±standard deviation;

FIG. 21 is a graph showing the release of ASX during in-vitro simulated digestion of NPs; the values are given as mean values±standard deviation;

FIGS. 22A-B are bar graphs showing the relative concentration of the different ASX esterification forms at time zero and after 60 min digestion in SGF and 120 min digestion in SIF of NPs (FIG. 22A) and H.p. oleoresin (FIG. 22B);

FIGS. 23A-B are pictures of the dissolution behavior of the granules containing curcumin particles, in water (FIG. 23A) and a picture of the appearance of the solution with a Becker illuminated from the bottom;

FIG. 24 is a microscope image of the granules containing curcumin particles taken by an optical microscope;

FIGS. 25A-B are pictures of the dissolution behavior of the granules containing Coenzyme Q10 particles, in water (FIG. 23A) and a picture of the appearance of the solution with a Becker illuminated from the bottom;

FIG. 26 is a microscope image of the granules containing Coenzyme Q10 particles taken by an optical microscope;

FIGS. 27A-B are pictures of the dissolution behavior of the granules containing beta-carotene particles in water (FIG. 23A) and a picture of the appearance of the solution with a Becker illuminated from the bottom;

FIG. 28 is a microscope image of the granules containing beta-carotene particles taken by an optical microscope;

FIGS. 29A-B are pictures of the dissolution behavior of the granules containing fish oil particles in water (FIG. 23A) and a picture of the appearance of the solution with a Becker illuminated from the bottom;

FIG. 30 is a microscope image of the granules containing fish oil particles taken by an optical microscope;

FIGS. 31A-B are pictures of the dissolution behavior of the granules containing phytosterol particles in water (FIG. 23A) and a picture of the appearance of the solution with a Becker illuminated from the bottom;

FIG. 32 is a microscope image of the granules containing phytosterol particles taken by an optical microscope;

FIG. 33 are pictures of the dissolution behavior of the powder containing caffeine particles in water;

FIG. 34 is a microscope image of the powder containing caffeine particles taken by an optical microscope;

FIG. 35 are pictures of the dissolution behavior of the powder containing epigallocatechin gallate particles in water;

FIG. 36 is a microscope image of the powder containing epigallocatechin gallate particles taken by an optical microscope;

FIG. 37 is a graph showing DLS analysis of the NPs emulsion obtained with WPC containing 3% of caffeine; the size distribution is reported by number as %;

FIG. 38 is a graph showing DLS analysis of the NPs powder obtained with WPC containing 3% of caffeine; the size distribution is reported by number as %;

FIG. 39 is a bar graph showing the cell viability of HepG2 cells incubated at different concentrations of H.p. oleoresin and WPC ASX NPs;

FIGS. 40A-D are graphs showing the cells fluorescence unit variance in response to different radical generator;

FIGS. 41A-B are graphs showing the cellular antioxidant activity tested in adult mice macrophages cells (J774A.1) via flow cytometry with WPC ASX NPs, H.p. oleoresin and WPC (FIG. 41A) and the comparison between the antioxidant properties of Trolox and WPC ASX NPs (FIG. 41B);

FIG. 42 are micrograph pictures obtained by confocal microscope of HepG2 and Caco2 cells incubated for different times with WPC ASX NPs labelled with the use of fluorescein isothiocyanate (FITC);

FIGS. 43A-B are graphs showing the cellular uptake inhibition of WPC ASX NPs in presence of a blocking condition in HepG2 (FIG. 43A) and Caco2 cells (FIG. 43B);

FIGS. 44A-B are graphs showing the variation of size and PDI (FIG. 44A) and z-potential (FIG. 44B) as a consequence of the different protein concentrations used to produce ASX soya protein isolate (SPI) NPs. Differences between values indicated by the same letter are statistically significant (P<0.05);

FIGS. 45A-B are graphs showing the variation of size and PDI (FIG. 45A) and z-potential (FIG. 45B) as a function of the different H.p. oleoresin concentrations used to produce ASX SPI NPs. Differences between values indicated by the same letter are statistically significant (P<0.05);

FIGS. 46A-B are pictures of the appearance of ASX SPI NPs as a function of the different protein concentrations (FIG. 46A) and H.p. oleoresin concentrations (FIG. 46B);

FIGS. 47A-B are graphs showing the variation of size and PDI (FIG. 47A) and z-potential (FIG. 47B) as a consequence of the different protein concentrations used to produce ASX pea protein isolate (PPI) NPs. Differences between values indicated by the same letter are statistically significant (P<0.05). Capital letter correspond to the significance of PDI;

FIGS. 48A-B are graphs showing the variation of size and PDI (FIG. 48A) and z-potential (FIG. 48B) as a consequence of the different H.p. oleoresin concentrations used to produce ASX PPI NPs. Differences between values indicated by the same letter are statistically significant (P<0.05). Capital letter correspond to the significance of PDI;

FIG. 49 is a picture of the appearance of ASX SPI NPs produced with differently treated proteins: H (Heat), N (non-treated), pH and pH+H (pH+heat);

FIGS. 50A-B are graphs showing the dependence of ASX SPI NPs size and PDI (FIG. 50A) and Z-potential (FIG. 50B) on the different protein treatments; differences between values indicated by the same letter are statistically significant (P<0.05);

FIG. 51 is a picture of the appearance of ASX PPI NPs produced with the differently treated proteins: H (Heat), N (non-treated), pH and pH+H (pH+heat);

FIGS. 52A-B are graphs showing the dependence of ASX PPI NPs size and PDI (FIG. 52A) and Z-potential (FIG. 52B) on the different protein treatments;

FIG. 53 is a picture of the appearance of the ASX suspensions using rice protein isolate RPI as stabilizer;

FIG. 54 is a graph showing the ASX release from ASX WPC NPs, ASX SPI NPs and ASX PPI NPs during in-vitro simulated digestion;

FIGS. 55A-B are optical microscope images showing the agglomeration of ASX NPs during gastric stage (FIG. 55A) and (FIG. 55B) their disappearance in the intestinal stage;

FIGS. 56A-C are pictures of sunflower oil non encapsulated (FIG. 56A), sunflower oil NPs preparation (50% w/w) (FIG. 56B), and sunflower oil NPs preparation (71% w/w) (FIG. 56B); and

FIG. 57 is a graph showing the particle size distribution of NPs produced with hydrolyzed proteins.

### DETAILED DESCRIPTION OF THE INVENTION

According to some embodiments, the present invention provides a particle comprising at least one compound having a protein-based shell at least partially surrounding the at least one compound. In some embodiments, a protein-based shell is at least partially surrounding a plurality of compounds. In some embodiments, a particle comprises a protein-based shell at least partially surrounding a bioactive compound. In some embodiments, a protein-based shell is at least partially surrounding a plurality of bioactive compounds. In some embodiments, a particle according to the present invention comprises a coating. In some embodiments a coating comprises a polysaccharide. In some embodiments, a coating is encapsulating a shelled compound. In some embodiments, a coating is encapsulating a plurality of shelled compounds. In some embodiments, a compound is a lipophilic compound. In some embodiments, a compound is a bioactive compound.

According to some embodiments, the present invention provides a particle comprising (i) at least one compound having a protein-based shell at least partially surrounding the at least one compound, and (ii) a coating comprising a polysaccharide encapsulating the at least one shelled compound.

According to some embodiments, the present invention provides a particle comprising at least one compound and at least one protein-based shell. In some embodiments, the present invention provides a particle comprising at least one compound, at least one protein-based shell and at least one cationic polymer. In some embodiments, the present invention provides a particle comprising at least one compound, at least one protein-based shell, at least one cationic polymer and at least one coating. In some embodiments, a coating is encapsulating the at least one compound, at least one protein-based shell and at least one cationic polymer. In some embodiments, a compound is a bioactive compound.

### The Particle

In some embodiments, the present invention provides a particle having a diameter of about 5 nm to 10000 nm, 5 nm to 9000 nm, 5 nm to 1000 nm, 5 nm to 900 nm, 5 nm to 700 nm, 5 nm to 500 nm, 5 nm to 300 nm, 10 nm to 10000 nm, 10 nm to 9000 nm, 10 nm to 1000 nm, 10 nm to 900 nm, 10 nm to 700 nm, 10 nm to 500 nm, 10 nm to 300 nm, 30 nm to 10000 nm, 30 nm to 9000 nm, 30 nm to 1000 nm, 30 nm to 900 nm, 30 nm to 700 nm, 30 nm to 500 nm, 30 nm to 300 nm, 50 nm to 5000 nm, 50 nm to 4000 nm, 50 nm to 3000 nm, 50 nm to 2000 nm, 50 nm to 1000 nm, 50 nm to 900 nm, 50 nm to 700 nm, 50 nm to 500 nm, or 50 nm to 300 nm, including any range therebetween.

In some embodiments, the present invention provides a particle having a diameter of about 1 μm to 500 μm, about 1 μm to 500 μm, about 1 μm to 400 μm, about 1 μm to 300 inn, about 1 μm to 250 μm, about 1 μm to 200 μm, about 1 μm to 100 μm, about 1 μm to 50 μm, about 50 μm to 500 μm, about 100 μm to 500 μm, about 150 μm to 500 μm, or about 50 μm to 300 μm, including any range therebetween.

In some embodiments, a protein-based shell has a thickness of about 1 nm to 30 nm, about 2 nm to 30 nm, about 3 nm to 30 nm, about 4 nm to 30 nm, about 5 nm to 30 nm, about 5 nm to 25 nm, about 5 nm to 20 nm, about 1 nm to 25 nm, about 1 nm to 20 nm, about 1 nm to 18 nm, or about 1 nm to 15 nm, including any range therebetween.

In some embodiments, a coating has a thickness of about 1 nm to 30 nm. In some embodiments, a coating has a thickness of about 2 nm to 30 nm, about 3 nm to 30 nm, about 4 nm to 30 nm, about 5 nm to 30 nm, about 5 nm to 25 nm, about 5 nm to 20 nm, about 1 nm to 25 nm, about 1 nm to 20 nm, about 1 nm to 18 nm, or about 1 nm to 15 nm, including any range therebetween.

In some embodiments, a compound and a protein-based shell have a diameter of about 5 nm to 300 nm. In some embodiments, a compound and a protein-based shell have a diameter of about 10 nm to 300 nm, about 20 nm to 280 nm, about 50 nm to 280 nm, about 50 nm to 250 nm, about 50 nm to 230 nm, about 50 nm to 200 nm, about 50 nm to 180 nm, about 50 nm to 160 nm, about 50 nm to 150 nm, about 50 nm to 130 nm, about 50 nm to 100 nm, about 70 nm to 200 nm, about 70 nm to 250 nm, about 100 nm to 250 nm, or about 100 nm to 300 nm, including any range therebetween.

In some embodiments, a protein-based shell partially surrounding a compound is of at least 85% of the total surface of compound. In some embodiments, a protein-based shell is partially surrounding a compound at least 87%, at least 90%, at least 93%, at least 95%, at least 98%, or at least 99% of the total surface of bioactive compound, including any value therebetween. In some embodiments, a protein-based shell is partially surrounding a compound about 85% to 100% of the total surface of the compound. In some embodiments, a protein-based shell is partially surrounding a compound about 85% to 99%, 85% to 98%, 85% to 95%, 85% to 90%, 85% to 89%, or 85% to 70% of the total surface of the compound, including any range therebetween. In some embodiments, a protein-based shell partially surrounding a compound is of at least 85% of the total mass of nanoparticle. In some embodiments, a protein-based shell is partially surrounding a compound at least 87%, at least 90%, at least 93%, at least 95%, at least 98%, or at least 99% of the total mass of nanoparticle, including any value therebetween. In some embodiments, a protein-based shell is partially surrounding a compound about 85% to 100% of the total mass of nanoparticle. In some embodiments, a protein-based shell is partially surrounding a compound about 85% to 99%, 85% to 98%, 85% to 95%, 85% to 90%, 85% to 89%, or 85% to 70% of the total mass of nanoparticle, including any range therebetween.

In some embodiments, the particle comprises 1% to 80% (w/w), 1% to 70% (w/w), 1% to 60% (w/w), 1% to 50% (w/w), 1% to 40% (w/w), 2% to 40% (w/w), 5% to 40% (w/w), 10% to 70% (w/w), 10% to 40% (w/w), 15% to 40% (w/w), 25% to 40% (w/w), 1% to 35% (w/w), 1% to 25% (w/w), 1% to 20% (w/w), 1% to 15% (w/w), 1% to 10% (w/w), 5% to 70% (w/w), 5% to 55% (w/w), 5% to 35% (w/w), 5% to 25% (w/w), 5% to 20% (w/w), 5% to 15% (w/w), or 5% to 10% (w/w), of the compound, including any range therebetween.

In some embodiments, the particle comprises 0.1% to 99% (w/w), 0.1% to 97% (w/w), 0.1% to 95% (w/w), 0.1% to 90% (w/w), 0.1% to 50% (w/w), 0.1% to 30% (w/w), 0.5% to 30% (w/w), 0.9% to 30% (w/w), 1% to 99% (w/w), 1% to 97% (w/w), 1% to 95% (w/w), 1% to 90% (w/w), 1% to 50% (w/w), 1% to 30% (w/w), 1% to 20% (w/w), 1% to 15% (w/w), 1% to 10% (w/w), 0.1% to 15% (w/w), 0.1% to 10% (w/w), 5% to 30% (w/w), 5% to 25% (w/w), 5% to 20% (w/w), 5% to 15% (w/w), or 5% to 10% (w/w), of the protein-based shell, including any range therebetween. In some embodiments, the protein-based shell content depends on the final concentration of the encapsulated compound needed. If a low concentration of compound is needed, the protein-based shell content can be increased up to 99.9%.

In some embodiments, the concentration of a compound in a particle is about 0.01 mg/g to 500 mg/g. In some embodiments, the concentration of a compound in a particle is about 0.01 mg/g to 450 mg/g, about 0.01 mg/g to 400 mg/g, about 0.01 mg/g to 350 mg/g, about 0.01 mg/g to 300 mg/g, about 0.01 mg/g to 250 mg/g, about 0.01 mg/g to 200 mg/g, 0.01 mg/g to 180 mg/g, about 0.01 mg/g to 150 mg/g, about 0.01 mg/g to 100 mg/g, about 0.01 mg/g to 80 mg/g, about 0.01 mg/g to 50 mg/g, about 0.01 mg/g to 30 mg/g, about 0.01 mg/g to 20 mg/g, about 0.01 mg/g to 10 mg/g, about 0.5 mg/g to 200 mg/g, about 0.5 mg/g to 150 mg/g, about 0.05 mg/g to 50 mg/g, about 0.1 mg/g to 5 mg/g, about 0.5 mg/g to 5 mg/g, about 0.5 mg/g to 3 mg/g, about 1 mg/g to 100 mg/g, about 1 mg/g to 50 mg/g, about 1 mg/g to 30 mg/g, or about 1 mg/g to 5 mg/g, including any range therebetween.

In some embodiments, the at least one compound is soluble in an organic solvent.

In some embodiments, a particle as described herein is stable when in solution at a pH of more than 6. In some embodiments, a particle as described herein is stable when in solution at a pH of more than 6.5, more than 6.7, more than 7, more than 7.5, more than 8, more than 8.5, more than 9, more than 9.5, more than 10, more than 10.5, or more than 11, including any value therebetween.

In some embodiments, a compound is a bioactive compound. In some embodiments, a bioactive compound is a lipophilic compound. In some embodiments, a compound is one or more lipophilic compound, volatile organic compound, fragrance, protein, aroma, vitamin, lipophilic metabolite, partially lipophilic metabolite, or any combination thereof. In some embodiments, a compounds comprises astaxanthin, curcumin, omega 3, caffeine, beta-carotene, fish oil, sunflower oil, phytosterol, epigallocatechin gallate, Coenzyme Q10, vitamin D, cannabinoid (e.g. cannabidiol (CBD), tetrahydrocannabinol (THC)), or any functional derivative thereof, or any combination thereof.

As used herein, the term “lipophilic compound” refers to compounds and substances that do not dissolve in water and have the ability to dissolve in non-polar substances such as lipids. Lipophilic substances can be characterized by having an affinity for oil or fat, or being at least partially soluble in organic solvents.

As used herein, the terms “bioactive compound” and “bioactive agent” are used interchangeably to refer to a compound having a beneficial effect on the human or animal metabolism. In some embodiments, the bioactive compound is obtained, extracted, enriched or purified starting from a plant, microorganism, yeast or product of animal origin.

As used herein, the term “obtained” refers to a bioactive product that is directly available commercially. The term “extracted” refers to a bioactive principle that has been extracted. The term “enriched” refers to a bioactive product where the non-bioactive compounds have been separated as much as possible. The term “purified” refers to a bioactive product where only the bioactive compound is recovered.

In some embodiments, the bioactive compound is selected from the group consisting of carotenoids, cannabinoids, fatty acids, polyphenols, lipophilic substances, vitamins, lipophilic vitamins, flavonoids, isoflavones, curcuminoids, ceramides, pro-anthocyanidins, terpenoids, sterols, phytosterols, essential oils, edible oils and fractions, tocopherols and tocotrienols, lipophilic tetrapyrroles, sterol esters, squalene and retinoids, gum resins, or any combination thereof.

In some embodiments, the bioactive compound is astaxanthin. In some embodiments, the bioactive compound is omega 3. In some embodiments, the bioactive compound is curcumin. In some embodiments, the bioactive compound is beta-carotene. In some embodiments, the bioactive compound is fucoxanthin. In some embodiments, the bioactive compound is flax seed oil. In some embodiments, the bioactive compound is fish oil. In some embodiments, the bioactive compound is cannabidiol (CBD) containing extract. In some embodiments, the bioactive compound is catechin.

In some embodiments, the particles and compositions as described herein, comprise in some embodiments, cannabidiol (CBD), or any functional derivative thereof (i.e. a CBD derivative possessing similar, equivalent, or increased efficacy).

The phrase “CBD or any functional derivative thereof”, according to some embodiments, refers to compounds and/or compositions that comprise at least 80% CBD or any functional derivative thereof, at least 90% CBD or any functional derivative thereof, at least 92% CBD or any functional derivative thereof, at least 95% CBD or any functional derivative thereof, at least 97% CBD or any functional derivative thereof, or at least 99% CBD or any functional derivative thereof, including any value therebetween. In some embodiments, CBD or any functional derivative thereof, comprises tetrahydrocannabinol (THC).

As used herein, the term “cannabinoid” includes naturally occurring and non-natural derivatives of cannabinoids which can be obtained by derivation of natural cannabinoids. The cannabinoid used in the formulations of the invention is natural, semi-synthetic, or synthetic. The cannabinoid is included in its free form, or in the form of a salt; an acid addition salt of an ester; an amide; an enantiomer; an isomer; a tautomer; a prodrug; a derivative of an active agent of the present invention; different isomeric forms (for example, enantiomers and diastereoisomers), both in pure form and in admixture, including racemic mixtures; enol forms. The term “cannabinoid” is also meant to encompass derivatives that are produced from another compound of similar structure by the replacement of, e.g., substitution of one atom, molecule or group by another such as 11-hydroxy-delta-8-tetrahydrocannabinol and 11-hydroxy-delta-9-tetrahydrocannabinol. The term “cannabinoid”, as used in the present invention, further includes delta-8-tetrahydrocannabinol, delta-9-tetrahydrocannabinol, cannabidiol, cannabinol, cannabigerol, nabilone, delta-9-tetrahydro cannabinotic acid, the non-psychotropic cannabinoid 3-dimethylnepty 11 carboxylic acid homologine 8. (J. Med. Chem. 35, 3135, 1992 herein incorporated by reference in its entirety). The term cannabinoid also includes prodrugs of cannabinoids, as well as pharmaceutically acceptable salts and complexes of cannabinoids. An example of a suitable prodrug is THC-hemisuccinate. The term “cannabinoid” is further meant to encompass natural cannabinoids that have been purified or modified, and synthetically derived cannabinoids.

In one embodiment, a particle or composition as described herein comprises a CBD extract. In one embodiment, a particle or composition as described herein comprises CBD oleoresin. In one embodiment, a particle or composition as described herein comprises a plant material. In one embodiment, a composition as described herein comprises CBD enriched plant material.

As used herein, the term “plant material” refers to whole plants, plant extracts and also parts thereof which contain the principal medically active constituents, for example the aerial parts of the plant or isolated leaves, stems, flowering heads, fruits or roots.

In one embodiment, plant material refers to any plant material known to contain cannabinoids. In one embodiment, the plant material is derived from one or more cannabis plants.

The term “Cannabis plant (s)” encompasses wild type Cannabis sativa and also variants thereof, including cannabis chemovars which naturally contain different amounts of the individual cannabinoids, Cannabis sativa subspecies indica including the variants var. indica and var. kafiristanica, Cannabis indica and also plants which are the result of genetic crosses, self-crosses or hybrids thereof. The term “Cannabis plant material” is to be interpreted accordingly as encompassing plant material derived from one or more cannabis plants. For the avoidance of doubt, it is hereby stated that “cannabis plant material” includes dried cannabis biomass.

Non-limiting examples of astaxanthin used under the compositions and methods of the present inventions include the free form of astaxanthin, trans-astaxanthin, 9-cis and 13-cis-astaxanthin isomeric forms, astaxanthin fatty acid monoesters, and astaxanthin fatty acid diesters. Unless otherwise noted, these components are collectively referred to as “astaxanthin” herein. Examples of fatty acids in astaxanthin fatty acid esters include lauric acid, myristic acid, pentadecanoic acid, palmitic acid, palmitoleic acid, heptadecanoic acid, elaidic acid, ricinoleic acid, petroselinic acid, vaccenic acid, eleostearic acid, punicic acid, licanic acid, parinaric acid, gadoleic acid, 5-eicosenoic acid, 5-docosenoic acid, cetoleic acid, erucic acid, 5,13-docosaclienoic acid, selacholeic acid, decenoic acid, dodecenoic acid, oleic acid, stearic acid, eicosapentaenoic acid, docosahexaenoic acid, linoleic acid, α-linolenic acid, and arachidonic acid.

In some embodiments, astaxanthin of the present invention is produced synthetically. In some embodiments, astaxanthin of the present invention is natural astaxanthin. In some embodiments, astaxanthin of the present invention is obtained from microscopic plants. In another embodiment, the plant is the micro-alga Haematococcus pluvialis. In some embodiments, astaxanthin of the present invention is a mixture of synthetically produced astaxanthin and natural astaxanthin. Each possibility represents a separate embodiment of the present invention.

In some embodiments, a protein-based shell comprises whey protein, soya protein, pea protein, fava bean protein, collagen or any combination thereof.

In some embodiments, a protein-based shell comprises a protein. In some embodiments, a protein-based shell comprises whey protein. In some embodiments, a protein-based shell comprises collagen.

As used herein, the terms “peptide”, “polypeptide” and “protein” are used interchangeably to refer to a polymer of amino acid residues. The terms “peptide”, “polypeptide” and “protein” as used herein encompass native peptides, peptidomimetics (typically including non-peptide bonds or other synthetic modifications) and the peptide analogs peptoids and semi-peptoids or any combination thereof. In another embodiment, the terms “peptide”, “polypeptide” and “protein” apply to amino acid polymers in which at least one amino acid residue is an artificial chemical analog of a corresponding naturally occurring amino acid.

In some embodiments, the polypeptide of the protein extract described herein, is selected from, without being limited thereto, an animal protein, a plant protein, or an algae protein. In some embodiments, the polypeptide of the protein extract described herein is selected from: a purified protein, a concentrated protein, an isolated protein fraction, a protein hydrolysate, or any combination thereof.

As used here, the term “protein hydrolysate” includes all hydrolyzed products of proteins prepared by using a proteolytic enzyme preparation, a microorganism containing suitable proteolytic activity or acid hydrolysis or any combination thereof, and having serum lipid profile improving effect. Commercially available hydrolysates can be used, or hydrolysates can be prepared. In some embodiments, hydrolysates have a molecular weight of 300-100 000 Da. In some embodiments, hydrolysates have a molecular weight of 500-50 000 Da. In some embodiments, hydrolysates have a molecular weight of 500-30 000 Da. In some embodiments, hydrolysates are only slightly soluble in water.

Plant, animal or microbial proteins and/or their mixtures can be used as protein sources for the hydrolysates. In some embodiments, the protein is of vegetable origin. In some embodiments, the protein is of grain or legume origin. Suitable vegetable protein sources are for example soybean protein, wheat protein, wheat gluten, corn protein, oat protein, rye protein, rice protein, rapeseed or canola protein, barley protein, flaxseed protein, potato protein, pea protein, lupin protein, sunflower protein, hemp protein, fava bean protein and buckwheat protein.

In some embodiments, the protein is of animal origin. Suitable animal protein sources are for example milk proteins, such as caseins and whey protein, and their fractions, egg proteins, collagens and gelatins. In some embodiments, proteins can be used in different commercially available purified or non-purified forms as source for the hydrolysates. In some embodiments, materials containing these proteins and other major constituents, such as carbohydrates, are used as source for the hydrolysates.

In some embodiments, the protein extract is a plant protein. In some embodiments, the plant protein is extracted from, without being limited thereto, potato, pea, soy, chickpea, quinoa, wheat, lentils, fava or bean. In some embodiments, the plant protein is extracted from a potato. In some embodiments, the plant protein is extracted from a pea. In some embodiments, the plant protein is extracted from a chickpea.

In some embodiments, a protein extract is an animal protein. In some embodiments, the protein is extracted from, without being limited thereto, a mammal, a bird or an insect. In some embodiments, the protein is selected from an egg protein or a whey protein. In some embodiments, the protein is whey protein.

As used herein, the term “whey protein” refers to a product of dairy origin, which comes from the watery part of milk that separates from the curd, as in the process of making cheese, left over after butterfat, casein and albumin are removed. In some embodiments, “whey protein” refers to a product comprising at least 80% of whey proteins. In some embodiments, “whey protein” refers to a product comprising at least 85% of whey proteins. In some embodiments, “whey protein” refers to a product comprising at least 90% of whey proteins.

As used herein, the term “polysaccharide” refers to a large molecule made of many smaller monosaccharides connected via glycosidic bonds. Special enzymes bind these small monomers together creating large sugar polymers, or polysaccharides. A polysaccharide is also called a glycan. A polysaccharide can be a homopolysaccharide, in which all the monosaccharides are the same, or a heteropolysaccharide in which the monosaccharides vary. A molecule with a straight chain of monosaccharides is called a linear polysaccharide, while a chain that has arms and turns is known as a branched polysaccharide. In some embodiments, the term polysaccharide refers to gums, dextrans, celluloses, and heteropolysaccharides, and derivatives thereof, hydrolysates thereof, crosslinked products thereof and combinations thereof. In some embodiments, a polysaccharide is maltodextrin.

As used herein, the term “maltodextrin” refers to glucose polymers having a dextrose equivalent (DE) of less than 20. In some embodiments, maltodextrin have a DE less than or equal to 10. In some embodiments, maltodextrin have a DE of less than 5. The term “dextrose equivalent” refers to the reducing power (or the reducing sugar content) of starch hydrolysates calculated as dextrose (dextrose or glucose has a DE=100) on a dry weight basis. Maltodextrins having a high DE have lower molecular weights (are more highly converted) than those having a low DE Maltodextrin can be made from any suitable edible starch, e.g., starch from corn, rice, wheat, beets, potatoes, tapioca and sorghum.

Maltodextrin is typically generated by hydrolyzing a starch slurry with heat-stable α-amylase at temperatures at 85-90° C. until the desired degree of hydrolysis is reached and then inactivating the α-amylase by a second heat treatment. The maltodextrin can be purified by filtration and then spray dried to a final product. Maltodextrins are typically characterized by their dextrose equivalent (DE) value, which is related to the degree of hydrolysis defined as: DE=MW dextrose/number-averaged MW starch hydrolysate X 100. Generally, maltodextrins are considered to have molecular weights that are less than amylose molecules.

In some embodiments, a particle further comprises a cationic polymer interacting with at least a portion of a protein-based shell. In some embodiments, interacting is electrostatic interactions.

As used herein, the term “cationic polymer” refers to naturally and synthetically derived cationic polymers.

In some embodiments, cationic polymer comprises a cationic polysaccharide.

As used herein, the term “cationic polysaccharide” refers to polymers based on 5 or 6 carbon sugars and derivatives thereof which have been made cationic by engraphing of cationic moieties on the polysaccharide backbone. They may be composed of one type of sugar or of more than one type, i.e. copolymers of the above derivatives and cationic materials. The monomers may be in straight chain or branched chain geometric arrangements. Non-limiting examples of cationic polysaccharide polymers include the following: cationic celluloses and hydroxyethylcelluloses; cationic starches and hydroxyalkyl starches; cationic polymers based on arabinose monomers such as those which could be derived from arabinose vegetable gums; cationic polymers derived from xylose polymers found in materials such as wood, straw, cottonseed hulls, and corn cobs; cationic polymers derived from fucose polymers found as a component of cell walls in seaweed; cationic polymers derived from fructose polymers such as inulin found in certain plants; cationic polymers based on acid-containing sugars such as galacturonic acid and glucuronic acid; cationic polymers based on amine sugars such as galactosamine and glucosamine; cationic polymers based on 5 and 6 membered ring polyalcohols; cationic polymers based on galactose monomers which occur in plant gums and mucilages; cationic polymers based on mannose monomers such as those found in plants, yeasts, and red algae; cationic polymers based on the galactomannan copolymer known as guar gum obtained from the endosperm of the guar bean.

In some embodiments, cationic polymer comprises chitosan.

As used herein the term “chitosan” refers to a polymer of natural origin derived from chitin (poly-N-acetyl-D-glucosamine), where an important part of the acetyl groups of the N have been eliminated by hydrolysis. In some embodiments, the degree of deacetylation is greater than 40%. In some embodiments, the degree of deacetylation is greater than 60%. In some embodiments, the degree of deacetylation is in the range of 60% to 98%. Chitosan has an amino-polysaccharide structure and cationic character.

In some embodiments, chitosan used in the present invention is characterized by having a low molecular weight. In some embodiments, chitosan such as described above, or a derivative thereof has a molecular weight less than 90 kDa. In some embodiments, chitosan has a molecular weight in the range of 1 kDa to 90 kDa. In some embodiments, chitosan has a molecular weight in the range of 1 kDa to 75 kDa. In some embodiments, chitosan has a molecular weight in the range of 2 kDa to 50 kDa. In some embodiments, chitosan has a molecular weight in the range of 2 kDa to 30 kDa. In some embodiments, chitosan has a molecular weight in the range of 2 kDa to 15 kDa. The chitosan with this molecular weight is obtained by methods well known to a skilled person in the art, such as oxidative reduction of the chitosan polymer using different proportions of NaNO₂.

In some embodiments, an alternative to chitosan, or a derivative thereof is used in the present invention. In some embodiments, a chitosan has a molecular weight less 90 kDa wherein one or more hydroxyl groups and/or one or more amine groups have been modified, with the aim of increasing the solubility of the chitosan or increasing the adhesive nature thereof. These derivatives include, among others, acetylated, alkylated or sulfonated chitosans, thiolated derivatives.

In some embodiments, a chitosan derivative is selected from O-alkyl ethers, O-acyl esters, trimethyl chitosan, or chitosans modified with polyethylene glycol. Other possible derivatives are salts, such as citrate, nitrate, lactate, phosphate, glutamate, etc. In any case, a person skilled in the art knows how to identify the modifications which can be made on the chitosan without affecting the stability and commercial feasibility of the formulation.

In some embodiments, a cationic polymer interacts with at least a portion of a protein-based shell via electrostatic interactions.

In some embodiments, a particle further comprising a cationic polymer interacting with at least a portion of a protein-based shell, has a diameter of about 50 nm to 300 nm. In some embodiments, a particle further comprising a cationic polymer interacting with at least a portion of a protein-based shell, has a diameter of about 50 nm to 250 nm, 50 nm to 230 nm, about 50 nm to 200 nm, about 50 nm to 180 nm, about 50 nm to 160 nm, about 50 nm to 150 nm, about 50 nm to 130 nm, about 50 nm to 100 nm, about 70 nm to 200 nm, about 70 nm to 250 nm, about 100 nm to 250 nm, including any range therebetween.

In some embodiments, a particle as described herein is stable when in solution at a pH in the range of 1 to 5.5. In some embodiments, a particle as described herein is stable when in solution at a pH in the range of 1 to 5.5, 1 to 3, 1 to 2.5, or 1 to 3.5, including any range therebetween.

### The Composition

According to some embodiments, the present invention provides a composition comprising a plurality of particles as described elsewhere herein. In some embodiments, the present invention provides a composition comprising a plurality of particles comprising (i) at least one compound having a protein-based shell at least partially surrounding the at least one compound, and (ii) a coating comprising a polysaccharide encapsulating the at least one shelled compound.

In some embodiments, a composition as described herein, is an edible composition. In some embodiments, a composition is a cosmetic composition. In some embodiments, a composition as described herein, is a dietary supplement composition. In some embodiments, a composition as described herein, is a pharmaceutical composition. In some embodiments, a composition as described herein, is an agrochemical composition.

In some embodiments, a composition as described herein, is in the form of a powder. In some embodiments, a composition as described herein, is in the form of a granulate. In some embodiments, a composition as described herein, is in the form of an agglomerate. In some embodiments, the composition is in the form of a highly water-soluble or dispersible composition. In some embodiments a powder is soluble or dispersible in water at ambient temperature.

In some embodiments, a powder as described herein is stable at ambient temperature. In some embodiments, a powder as described herein is stable at 25° C. to 40° C. In some embodiments, a powder as described herein is stable at 25° C. to 40° C., 25° C. to 37° C., 25° C. to 35° C., 25° C. to 32° C., or 25° C. to 30° C., including any range therebetween.

In some embodiments, the composition comprises 1% to 80% (w/w), 1% to 70% (w/w), 1% to 60% (w/w), 1% to 50% (w/w), 1% to 40% (w/w), 2% to 40% (w/w), 5% to 40% (w/w), 10% to 70% (w/w), 10% to 40% (w/w), 15% to 40% (w/w), 25% to 40% (w/w), 1% to 35% (w/w), 1% to 25% (w/w), 1% to 20% (w/w), 1% to 15% (w/w), 1% to 10% (w/w), 5% to 70% (w/w), 5% to 55% (w/w), 5% to 40% (w/w), 5% to 35% (w/w), 5% to 25% (w/w), 5% to 20% (w/w), 5% to 15% (w/w), or 5% to 10% (w/w), of the at least one compound, including any range therebetween.

In some embodiments, a powder has a content of 0.5 mg/g to 500 mg/g of at least one compound. In some embodiments, a powder has a content of 0.5 mg/g to 450 mg/g, 0.5 mg/g to 400 mg/g, 0.5 mg/g to 350 mg/g, 0.5 mg/g to 300 mg/g, 0.5 mg/g to 250 mg/g, 0.5 mg/g to 200 mg/g, 0.5 mg/g to 150 mg/g, 0.5 mg/g to 100 mg/g, 0.5 mg/g to 50 mg/g, 0.5 mg/g to 30 mg/g, 0.5 mg/g to 20 mg/g, 0.5 mg/g to 10 mg/g, 0.5 mg/g to 5 mg/g, 0.5 mg/g to 15 mg/g, 0.5 mg/g to 8 mg/g, 0.5 mg/g to 7 mg/g, 0.5 mg/g to 5 mg/g, 0.5 mg/g to 3 mg/g, 0.5 mg/g to 2 mg/g, 0.5 mg/g to 1.5 mg/g, 0.5 mg/g to 1 mg/g, 0.8 mg/g to 2 mg/g, 1 mg/g to 5 mg/g, 1 mg/g to 10 mg/g, 1 mg/g to 20 mg/g, 1 mg/g to 50 mg/g, 1 mg/g to 100 mg/g, 1 mg/g to 250 mg/g, or 1 mg/g to 3 mg/g of at least one compound, including any range therebetween.

In some embodiments, at least 80% of particles of a composition as described herein, have a diameter in the rage of 5 nm to 300 nm. In some embodiments, at least 80%, at least 85%, at least 89%, at least 90%, at least 95% of particles of a composition as described herein, have a diameter in the rage of 5 nm to 300 nm.

In some embodiments, particles of a composition as described herein, have a diameter the rage of 5 nm to 300 nm when re-dispersed in water. In some embodiments, particles of a composition as described herein, have a diameter of about 5 nm to 280 nm, about 5 nm to 250 nm, about 5 nm to 230 nm, about 5 nm to 200 nm, about 5 nm to 180 nm, about 5 nm to 160 nm, about 5 nm to 150 nm, about 5 nm to 130 nm, about 5 nm to 100 nm, about 15 nm to 280 nm, about 15 nm to 250 nm, about 15 nm to 230 nm, about 15 nm to 200 nm, about 15 nm to 180 nm, about 15 nm to 160 nm, about 15 nm to 150 nm, about 15 nm to 130 nm, about 15 nm to 100 nm, about 50 nm to 280 nm, about 50 nm to 250 nm, about 50 nm to 230 nm, about 50 nm to 200 nm, about 50 nm to 180 nm, about 50 nm to 160 nm, about 50 nm to 150 nm, about 50 nm to 130 nm, about 50 nm to 100 nm, about 70 nm to 200 nm, about 70 nm to 250 nm, about 100 nm to 250 nm, or about 100 nm to 300 nm when re-dispersed in water, including any range therebetween. In some embodiments, the particles are in the form of powder or granules.

In some embodiments, a composition as described herein has a polydispersity index in the range of about 0.05 to 0.7. In some embodiments, a composition as described herein has a polydispersity index in the range of about 0.05 to 0.5, 0.05 to 0.3, about 0.05 to 0.25, about 0.08 to 0.3, about 0.1 to 0.3, about 0.12 to 0.3, or about 0.15 to 0.3, including any range therebetween.

In some embodiments, a composition as described herein has a zeta potential in the range of about −50 mV to −10 mV. In some embodiments, a composition as described herein has a zeta potential in the range of about −35 mV to −10 mV, about −34 mV to −10 mV, about −33 mV to −10 mV, about −50 mV to 0 mV, about −50 mV to −5 mV, about −35 mV to −5 mV, or about −35 mV to 0 mV, including any range therebetween.

In some embodiments, a composition as described herein has antioxidant activity. In some embodiments, a composition as described herein increases the antioxidant activity of a compound. In some embodiments, a compound is a bioactive compound. In some embodiments, a composition as described herein comprising an encapsulated compound has antioxidant activity 1 to 100 times higher than the compound non-encapsulated. In some embodiments, a composition as described herein comprising an encapsulated compound has antioxidant activity 1 to 90, 1 to 80, 1 to 70, 1 to 60, 1 to 50, 1 to 40, 1 to 30, 1 to 20, 1 to 15, 1 to 10, 1 to 9, 1 to 8, 1 to 7, 1 to 6, or 1 to 5 times higher than the compound non-encapsulated, including any range therebetween.

According to some embodiments, the present invention provides a composition comprising a plurality of particles as described elsewhere herein. In some embodiments, the present invention provides a composition comprising a plurality of particles comprising (i) at least one compound having a protein-based shell at least partially surrounding the at least one compound, and (ii) a coating comprising a polysaccharide encapsulating the at least one shelled compound, and a cationic polymer interacting with at least a portion of a protein-based shell.

In some embodiments, at least 80% of particles of a composition as described herein, have a diameter in the rage of 50 nm to 250 nm. In some embodiments, at least 80%, at least 85%, at least 89%, at least 90%, at least 95% of particles of a composition as described herein, have a diameter in the rage of 50 nm to 250 nm.

In some embodiments, particles of a composition as described herein, have a diameter in the rage of 50 nm to 250 nm. In some embodiments, particles of a composition as described herein, have a diameter of about 50 nm to 230 nm, about 50 nm to 200 nm, about 50 nm to 180 nm, about 50 nm to 160 nm, about 50 nm to 150 nm, about 50 nm to 130 nm, about 50 nm to 100 nm, about 70 nm to 200 nm, about 70 nm to 250 nm, or about 100 nm to 250 nm, including any range therebetween.

In some embodiments, a composition as described herein has a polydispersity index in the range of about 0.05 to 0.7. In some embodiments, a composition as described herein has a polydispersity index in the range of about 0.05 to 0.5, 0.05 to 0.3, about 0.05 to 0.25, about 0.08 to 0.3, about 0.1 to 0.3, about 0.12 to 0.3, or about 0.15 to 0.3, including any range therebetween.

In some embodiments, a composition as described herein has a zeta potential in the range of about 0 mV to 100 mV. In some embodiments, a composition as described elsewhere herein has a zeta potential in the range of about 0 mV to 80 mV, about 0 mV to 70 mV, about 0 mV to 60 mV, about 0 mV to 50 mV, about 0 mV to 45 mV, or about 0 mV to 40 mV, including any range therebetween.

As used herein the term “zeta potential” refers to a scientific term for electrokinetic potential in colloidal systems. In the colloidal chemistry literature, it is usually denoted using the Greek letter zeta, hence ζ-potential. Zeta potential is a measure of the magnitude of the repulsion or attraction between particles. Zeta potential is an index of the magnitude of interaction between colloidal particles and measurements of zeta potential are used to access the stability of colloidal systems.

In aqueous media, the pH of the sample affects its zeta potential. For example, if alkali is added to a suspension with a negative zeta potential the particles tend to acquire more negative charge. If sufficient acid is added to the suspension, then a point will be reached where the charge will be neutralized. Further addition of acid will cause a buildup of positive charge.

In some embodiments, a composition as described herein has a zeta potential at 25° C. In some embodiments, a composition as described herein has a zeta potential in the range of about 0.5 to 100 mV or about −0.5 to −100 mV. In some embodiments the zeta potential is in the range of about 1 to 60 mV or about −1 to −60 mV, about 14 to 50 mV or about −14 to −50 mV, about 30 to 50 mV or about −30 to −50 mV. In some embodiments the zeta potential is in the range of about 0.5 to 100 mV or about −0.5 to −100 mV, about 1 to 60 mV or about −1 to −60 mV, about 14 to 50 mV or about −14 to −50 mV, about 30 to 50 mV or about −30 to −50 mV, including any range therebetween.

In some embodiments, a compound is released from a particle in the intestinal phase under physiological conditions. In some embodiments, 20% to 90% of a compound is released in the intestinal phase under physiological conditions. In some embodiments, 20% to 85%, 20% to 60%, 30% to 90%, 40% to 90%, 50% to 90%, 50% to 85%, 50% to 80%, 50% to 75%, 50% to 70%, 55% to 90%, 60% to 90%, or 55% to 85%, of a compound is released in the intestinal phase under physiological conditions, including any range therebetween. In some embodiments, a bioactive compound is released from a particle in the intestinal phase under physiological conditions. In some embodiments, 20% to 90% of a bioactive compound is released in the intestinal phase under physiological conditions. In some embodiments, 20% to 85%, 20% to 60%, 30% to 90%, 40% to 90%, 50% to 90%, 50% to 85%, 50% to 80%, 50% to 75%, 50% to 70%, 55% to 90%, 60% to 90%, or 55% to 85%, of a bioactive compound is released in the intestinal phase under physiological conditions, including any range therebetween.

In some embodiments, the composition is a gastro-resistant composition. In some embodiments, the particles are gastro-resistant particles.

As used herein, the term “particle” refers to both nano-scale and micro-scale particles and, except where otherwise noted, is generally synonymous with the term “nanoparticle”.

In some embodiments, the nanoparticles as described herein are on the nanoscale. In some embodiments, nanoscale nanoparticles measure between 1 and 1000 nanometers in at least one measurable dimension. In some embodiments, the nanoparticles may measure greater than 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, or 240 nm in at least one measurable dimension. In some embodiments, nanoparticles may measure less than 250 nm, 240 nm, 230 nm, 220 nm, 210 nm, 200 nm, 190 nm, 180 nm, 170 nm, 160 nm, 150 nm, 140 nm, 130 nm, 120 nm, 110 nm, 100 nm, 90 nm, 80 nm, 70 nm, 60 nm, 50 nm, or 40 nm in at least one measurable dimension. In some embodiments, nanoparticles have various shapes, including rods, spheres, and platelets.

In some embodiments, the particle diameter is an average particle diameter. In some embodiments, the particle size is an average particle size. In some embodiments, particle size and particle diameter refers to Z-average. The term “average particle size” refers to a length dimension which is designated herein as Z-average, and as used herein refers to the intensity weighted mean hydrodynamic size of an ensemble collection of particles measured by dynamic light scattering (DLS). The Z average is derived from a cumulants analysis of a measured autocorrelation curve, wherein a single particle size is assumed and a single exponential fit is applied to the autocorrelation function. In some embodiments, the diameter is analyzed through number %, when is important to know the exact size of the particle without the influence of the signal of bigger particles that are present in low percentage in the sample.

In some embodiments, particles refer to the emulsion particles of the discontinuous phase. In some embodiments, the particles increase the bioavailability of incorporated bioactive agents. In some embodiments, a bioactive agent is a lipophilic agent. In some embodiments, the particles increase the bioavailability of an incorporated bioactive agent at the physiological target site due to their relatively large surface area and effective encapsulation of the bioactive compound. Bioactive agents which may be encapsulated in accordance with the present invention include pharmaceutical compositions or compounds, cosmetic formulations, nutraceutical compositions or compounds, nutritional components, or biologically active components.

In some embodiments, the present invention provides a composition comprising a compound which has a better bioavailability than when the compound is provided alone. In some embodiments, the present invention provides a composition comprising a bioactive compound which has a better bioavailability than when the compound is provided alone.

### The Method

According to some embodiments, the present invention provides a method for encapsulating a compound. In some embodiments, a compound is a bioactive compound. In some embodiments, the present invention provides a method for encapsulating a compound comprising the steps of a) mixing a compound and a solvent, b) mixing a compound and a solvent with a protein, or a polysaccharide, or both, thereby obtaining a nanoemulsion; c) evaporating a solvent thereby obtaining a particle; and d) drying a particle with a protein, a polysaccharide, or a mixture thereof, thereby encapsulating a compound. In some embodiments, the particle is a nanoparticle.

In some embodiments, the present invention provides a method for encapsulating a compound comprising the steps of a) mixing a compound and a solvent, b) mixing a compound and a solvent with a protein, or a polysaccharide, or both, thereby obtaining a nanoemulsion; c) evaporating a solvent thereby obtaining a nanoparticle; and d) drying a nanoparticle with a protein, a polysaccharide, or a mixture thereof, thereby encapsulating a compound.

In some embodiments, the method comprises the step of adding a cationic polymer prior to the drying.

In some embodiments, the compound is one or more lipophilic compound, volatile organic compound, fragrance, protein, aroma, vitamin, lipophilic metabolite, partially lipophilic metabolite, or any combination thereof. In some embodiments, the compound comprises astaxanthin, curcumin, omega 3, caffeine, beta-carotene, fish oil, phytosterol, epigallocatechin gallate, Coenzyme Q10, or any combination thereof.

In some embodiments, the drying is spray drying, granulating, agglomerating, or any combination thereof, the particles.

In some embodiments, a compound is in a suspension. In some embodiments, a compound is a suspension in a solvent.

In some embodiments, a solvent has a boiling point in the range of 35° C. to 80° C. In some embodiments, a solvent has a boiling point in the range of 35° C. to 79° C., 35° C. to 78° C., 37° C. to 80° C., 38° C. to 80° C., or 38° C. to 78° C., including any range therebetween. In some embodiments, a solvent comprises ethyl acetate. In some embodiments, a solvent comprises ethyl acetate, dichloromethane, pentane, chloroform, 1,4 dioxane, benzene, toluene, N-pentane, N-hexane, cyclohexane, or any combination thereof.

In some embodiments, a compound and a protein are used in a ratio of 4:1 to 1:50 (w/w). In some embodiments, a bioactive compound and a protein are used in a ratio of 3:1 to 1:50 (w/w), 2:1 to 1:50 (w/w), 1:1 to 1:50 (w/w), 4:1 to 1:40 (w/w), 4:1 to 1:35 (w/w), 4:1 to 1:30 (w/w), 4:1 to 1:25 (w/w), 4:1 to 1:20 (w/w), 4:1 to 1:10 (w/w), 2:1 to 1:40 (w/w), 2:1 to 1:35 (w/w), 2:1 to 1:30 (w/w), 2:1 to 1:25 (w/w), 2:1 to 1:20 (w/w), 2:1 to 1:10 (w/w), 1:0.1 to 1:5 (w/w), 1:0.1 to 1:4 (w/w), 1:0.1 to 1:3 (w/w), 1:0.1 to 1:2 (w/w), 0.1:0.1 to 0.1:10 (w/w), 0.5:0.1 to 0.5:10 (w/w), 0.5:0.1 to 1:10 (w/w). 0.1:0.8 to 0.1:10 (w/w), 0.1:0.9 to 0.1:10 (w/w), 0.1:1 to 0.1:10 (w/w), 0.1:0.8 to 0.1:9 (w/w), 0.1:0.8 to 0.1:8 (w/w), 0.1:0.8 to 0.1:7 (w/w), 0.1:0.8 to 0.1:6 (w/w), 0.1:0.8 to 0.1:5 (w/w), or 0.1:0.8 to 0.1:4 (w/w), including any range therebetween.

In some embodiments a protein is in a solution. In some embodiments, a protein is in an aqueous solution. In some embodiments, the concentration of a protein in an aqueous solution is 0.5% to 25% (w/w). In some embodiments, the concentration of a protein in an aqueous solution is 1% to 25% (w/w), 0.5% to 23% (w/w), 0.5% to 20% (w/w), 0.5% to 19% (w/w), 0.5% to 17% (w/w), 0.5% to 15% (w/w), 0.5% to 14% (w/w), 0.5% to 13% (w/w), 0.5% to 12% (w/w), 0.5% to 11% (w/w), or 0.5% to 10% (w/w), including any range therebetween. In some embodiments, the protein concentration depends on the final concentration of the encapsulated compound needed. If a low concentration of compound is needed, the protein-based shell concentration can be increased up to 99.9% (w/w).

In some embodiments, mixing a compound and a solvent with a protein is using an ultra-sonicator. In some embodiments, mixing a compound and a solvent with a protein is using an ultra-sonicator for 5 seconds to 30 minutes. In some embodiments, mixing a compound and a solvent with a protein is using an ultra-sonicator for 10 seconds to 30 minutes, 20 seconds to 30 minutes, 50 seconds to 30 minutes, 5 seconds to 10 minutes, 5 seconds to 20 minutes, 1 minute to 25 minutes, 1 minute to 20 minutes, 1 minute to 15 minutes, 1 minute to 10 minutes, or 1 minute to 5 minutes, including any range therebetween.

In some embodiments, mixing a compound and a solvent with a protein is using a high shear homogenizer. In some embodiments, mixing a compound and a solvent with a protein is using a high shear homogenizer at 17.500 rpm to 24000 rpm. In some embodiments, mixing compound and a solvent with a protein is using a high shear homogenizer for 5 minutes to 30 minutes. In some embodiments, mixing a compound and a solvent with a protein is using a high shear homogenizer for 5 minutes to 20 minutes, 5 minutes to 25 minutes, 5 minutes to 20 minutes, 5 minutes to 15 minutes, 1 minute to 10 minutes, or 1 minute to 5 minutes, including any range therebetween.

In some embodiments, mixing a compound and a solvent with a protein is using a combination of high shear homogenizer and an ultra-sonicator. In some embodiments, mixing a compound and a solvent with a protein is using a microfluidic mixer. In some embodiments, mixing a compound and a solvent with a protein is using a microfluidic mixer with pressures up to 3000 bar. In some embodiments, mixing a compound and a solvent with a protein is using membrane emulsification.

In some embodiments, the method comprises the step of adding a cationic polymer. In some embodiments, the method comprises the step of adding a cationic polymer prior to drying. In some embodiments the method comprises the step of adding a cationic polymer after step b). In some embodiments the method comprises the step of adding a cationic polymer after step b) and before step c). In some embodiments the method comprises the step of adding a cationic polymer to the nanoemulsion. In some embodiments the method comprises the step of adding a cationic polymer after step c). In some embodiments the method comprises the step of adding a cationic polymer after evaporating a solvent. In some embodiments, the cationic polymer comprises chitosan.

In some embodiments, evaporating a solvent is using a nitrogen flow, nitrogen flow in the dark, an evaporator, a rotary evaporator such as circulation evaporator, falling film evaporator, rising film evaporator, climbing and falling film plate evaporator, multiple-effect evaporator, agitated thin film evaporator air current, or any combination thereof.

In some embodiments, step c) can be skipped using a closed-loop spray drying system or similar instruments capable of condensing solvents.

In some embodiments, the method comprises the step of granulating or agglomerating the particles. In some embodiments, the step of granulating or agglomerating the particles solution is performed instead of spray drying. In some embodiments, the step of agglomerating the particles is performed after spray drying. In some embodiments, a granulate is obtained by wet granulation process. In some embodiments, a granulate is obtained by a wet granulation process after step c). In some embodiments, a granulate is obtained by a wet granulation process by using closed-loop instruments. As used herein, “wet granulation”, refers to any suitable wet granulation process known in the art. In some embodiments, wet granulation process is selected from the group consisting of fluidized bed granulation, mixing granulation, extruder granulation, disc granulation, and roller granulation.

In some embodiments, the method of the present invention has an encapsulation yield of 60% to 95%. In some embodiments, the method of the present invention has an encapsulation yield of 65% to 95%, 60% to 90%, 70% to 90%, 70% to 85%, 75% to 80%, or 75% to 90%, including any range therebetween.

In some embodiments, the method of the present invention has an encapsulation efficacy of 80% to 100%. In some embodiments, the method of the present invention has an encapsulation efficacy of 81% to 100%, 82% to 100%, 85% to 100%, 87% to 100%, 89% to 100%, 90% to 100%, 80% to 95%, or 80% to 90%, including any range therebetween.

In some embodiments, the concentration of a compound in a particle is about 0.01 mg/g to 500 mg/g. In some embodiments, the concentration of a compound in a particle is about 0.01 mg/g to 450 mg/g, about 0.01 mg/g to 400 mg/g, about 0.01 mg/g to 350 mg/g, about 0.01 mg/g to 300 mg/g, about 0.01 mg/g to 250 mg/g, about 0.01 mg/g to 200 mg/g, 0.01 mg/g to 180 mg/g, about 0.01 mg/g to 150 mg/g, about 0.01 mg/g to 100 mg/g, about 0.01 mg/g to 80 mg/g, about 0.01 mg/g to 50 mg/g, about 0.01 mg/g to 30 mg/g, about 0.01 mg/g to 20 mg/g, about 0.01 mg/g to 10 mg/g, about 0.5 mg/g to 200 mg/g, about 0.5 mg/g to 150 mg/g, about 0.05 mg/g to 50 mg/g, about 0.1 mg/g to 5 mg/g, about 0.5 mg/g to 5 mg/g, about 0.5 mg/g to 3 mg/g, about 1 mg/g to 100 mg/g, about 1 mg/g to 50 mg/g, about 1 mg/g to 30 mg/g, or about 1 mg/g to 5 mg/g, including any range therebetween.

In some embodiments, the method comprises the step of spray drying the particles solution with a protein. In some embodiments, the method comprises the step of spray drying a particle with a polysaccharide. In some embodiments, the method comprises the step of spray drying a particle with a polysaccharide comprising maltodextrin. In some embodiments, the method comprises the step of spray drying a particle with a polysaccharide in a concentration from 1% to 60% (w/w).

In some embodiments, the method comprises the step of spray drying a particle with a polysaccharide in a concentration from 1% to 50%, 1% to 40%, 1% to 30%, 1% to 20%, 1% to 10%, 1% to 9%, 1% to 8%, 1% to 7%, or 1% to 6% (w/w), including any range therebetween. In some embodiments, a particle is a nanoparticle.

According to some embodiments, the present invention provides a method for increasing bioavailability of a compound to a subject upon administration. According to some embodiments, the present invention provides a method for increasing bioavailability of a lipophilic bioactive compound to a subject upon administration.

As used herein, the term “spray drying” refers to a method of producing a dry powder from a liquid or slurry by rapidly drying with a hot gas. This method is used for drying of many thermally-sensitive materials such as foods and pharmaceuticals. Air is the heated drying medium; however, if the liquid is a flammable solvent such as ethanol or the product is oxygen-sensitive then nitrogen is used. All spray dryers use some type of atomizer or spray nozzle to disperse the liquid or slurry into a controlled drop size spray. The most common of these are rotary disks and single-fluid high pressure swirl nozzles. Atomizer wheels are known to provide broader particle size distribution, but both methods allow for consistent distribution of particle size. Alternatively, for some applications, two-fluid or ultrasonic nozzles are used. Depending on the process needs, drop sizes from 5 to 500 μm can be achieved with the appropriate choices. The most common applications are in the 100 to 200 μm diameter range. The dry powder is often free-flowing. The most common spray dryers are called “single effect” spray dryers as there is only one stream of drying air at the top of the drying chamber. In most cases the air is blown in co-current of the sprayed liquid. The powders obtained with such type of dryers are fine with a lot of dusts and a poor flowability. In order to reduce dust and increase the flowability of the powders, a new generation of spray dryers known as “multiple effect” spray dryers have been developed. Instead of drying the liquid in one stage, the drying is done in two steps: one at the top (as per single effect) and one for an integrated static bed at the bottom of the chamber. The integration of this fluidized bed allows, by fluidizing the powder inside a humid atmosphere, to agglomerate the fine particles and to obtain granules having commonly a medium particle size within a range of 100 to 300 μm. Because of this large particle size, these powders are free-flowing. The fine powders generated by the first stage drying can be recycled in continuous flow either at the top of the chamber (around the sprayed liquid) or at the bottom inside the integrated fluidized bed. The drying of the powder can be finalized on an external vibrating fluidized bed. The hot drying gas is passed as a co-current or counter-current flow to the atomizer direction. The co-current flow enables the particles to have a lower residence time within the system and the particle separator (typically a cyclone device) operates more efficiently. The counter-current flow method enables a greater residence time of the particles in the chamber and usually is paired with a fluidized bed system. Alternatives to spray dryers include electrostatic spray dryers, freeze dryers, drum dryers, and pulse combustion dryers.

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion. Materials and methods

### Chemical and Reagents

Whey protein concentrate (WPC) 80% was gently provided by I.T.ALI. srl. (Italy). The protein content was 80% (w/w). Haematococcus pluvialis dried cells and oleoresin were obtained from Algatechnology (Israel). Maltodextrin DE 19 was from Agrana (Austria), whey protein isolate (WPI, Isolac) from Carbery (Ireland). Capsul and HICAP modified starch from Ingredion Incorporated (US). Starch, gum arabic ethyl acetate, HPLC-grade acetone, low molecular weight chitosan, pepsin, pancreatin, trypsin, sodium cholate were purchased from Sigma-Aldrich (US). All enzymes were of porcine origin.

Astaxanthin Extraction from H.p.

H.p. oleoresin was obtained using the protocol proposed by Bustos-Garza and co-workers with minor modifications. Briefly, the algae powder was pretreated by mixing 5 g of algae and 1 ml of 3 M HCl and treating the sample in a microwave oven for 1 min at 100 W. The pretreated algae were extracted with 25 ml of ethyl acetate in a tube with a screw cap, for 60 min under agitation at 50° C. in a thermal bath. The solid portion was separated by centrifugation at 3000 g for 10 min to eliminate the biomass. The oleoresin was dried by rotary evaporator (Buchi, Switzerland) and in the dark at 4° C. until use.

### Spectrophotometric Analysis

Quantification of ASX was performed using a UV/VIS spectrophotometer (Unicam UV2). The samples were diluted in ethyl acetate and the absorbance measured at 480 nm. The concentration of ASX was calculated following the equation:

$$[ A ] = \frac{10 \times {A}_{480} \times DF}{{E}_{( 1 \% ; 1 cm )} \times d}$$

Where [A] is the concentration of ASX expressed as mg/ml; A480 the sample's absorbance at 480 nm; DF: dilution factor; E(1%; 1 cm): ASX percent solution extinction coefficient [(g/100 ml)-1 cm-1] in ethyl acetate (2150); d: the optical path (cm).

### Turbidity Analysis

Turbidimetric analysis was performed by monitoring the absorbance at 660 nm. HPLC analysis

Reverse phase HPLC of astaxanthin-containing samples was performed with a Beckman System Gold (Beckman Coulter) on a C30 column (4.6×250 mm, particle size 5 um) (YMC Europe, Schermbeck, Germany) following a previously described method with minor modifications. The absorbance was monitored at 480 nm by a Beckman 168 diode array detector. The injection volume was 50 The elution was carried out at a flow rate of 1 ml/min using acetone (solvent A) and water (solvent B) as follows: isocratic elution at 84:16 (A:B) for 10 min and a gradient to 97:3 (A:B) for 100 min.

### Astaxanthin Nanoparticles Preparation

Astaxanthin nanoparticles (ASX NPs) were produced following a method described with some modifications. Whey protein concentrate (WPC) was dissolved in distilled water in a concentration range between 1 and 10%. The solution was stirred for 30 minutes at room temperature without pH modification. H.p. extract was differently diluted in ethyl acetate and combined with the protein solution at a ratio of 9:1 (protein solution:extract). A fine emulsion was produced using an ultra-sonicator for 10 minutes at a potency of 10 W (Microson ultrasonic cell disruptor XL). At the end of the process ethyl acetate was removed using a nitrogen flow in the dark. The WPC ASX NPs were kept in the dark at 4° C. until use.

In order to obtain an additional layer, the produced nanoparticles could be mixed with an equal volume of a cationic polymer solution such as chitosan at a concentration ranging from 0.01 to 0.5%, where chitosan was previously dissolved in a solution containing acetic acid. The contact between the negative shell constituted by WPC and the positive charge of chitosan give instantaneously the formation of a shell due to the electrostatic interaction between opposite charge.

In order to obtain a water dispersible powder ASX oleoresin was dissolved in ethyl acetate. The encapsulant matrices were rehydrated in deionized water for at least 1-hour prior the use, to allow a complete dissolution of the polymers. ASX oleoresin was diluted in ethyl acetate and combined with the polymer solution at a ratio of 9:1 (polymer solution:organic phase). A fine emulsion was produced using an ultra-sonicator for 10 minutes at a power of 12 W (Microson ultrasonic cell disruptor XL). At the end of the process ethyl acetate was removed using a rotavapor system. Further polymers (e.g. MD) can be added later before the atomization. The drying process was performed with the use of a Buchi Mini-Spray dryer B-290 (Switzerland). The condition used were as follows: drying air flow rate 40 m³/h; inlet air temperature 180±3; outlet air temperature 100±3 and a feed flow rate of 4 mL/min. The formed microparticles were collected in the collector at the bottom of the cyclone separator. The ASX particles powder was kept in alumina sealed bags and stored at 4° C. until use.

The NPs after evaporation can alternatively undergo a wet granulation process. Wet granulation, according to the present invention, may be any suitable wet granulation process selected from the group consisting of fluidized bed granulation, mixing granulation, extruder granulation, disc granulation, and roller granulation.

The granulation process was conducted by a fluid bed granulator (Mini-Glatt fluid bed system, Germany). Fifty grams of MD (used as seeds) were fluidized by a flowing stream of air of 6 m3/h, the temperature was set at 65° C. After preheating the powder for 5 minutes, 100 ml of ASX NPs was injected by a peristaltic pump at a speed of 1 ml/min. The liquid was top-sprayed on MD. The air pressure of the nozzle was set to 1 bar. During the process the stream of air was raised from 6 to 19 m3/h, to allow the fluidization of the growing particles. The product was left drying at 45° C. for 10 minutes until water activity of 0.2-0.25 was reached.

### Characterization of Astaxanthin Nanoparticles

### Surface Charge and Average Diameter

Zeta-potential, mean diameter and poly dispersity index (PDI) of ASX nanoparticles were analyzed by dynamic light scattering principles using a Malvern Zetasizer (Nano-ZS; Malvern Instruments, Worcestershire, U.K). Prior the analysis the sample were diluted 80 times to avoid multiple scattering effect. PDI value ranging from 0 to 1, indicated the distributions of the particle sizes, value close to 0 indicated a uniform population of particles and a value close to 1 indicated a wide variety of dimensions among particles size. Zeta potential give an important information about particles stability, for values closer to 0 the system is considered not stable, due to the absence of a net charge that can contrast the aggregation process of NPs. The obtained results are the average of at least three measurements.

### Encapsulation Efficiency

ASX NPs were treated enzymatically with 2 mg/ml of trypsin for 4 h in 0.1 M PBS 10 mM at 37° C. in a thermo-shaker (Biosan). The enzymatically digested solution was mixed with a double volume of ethyl acetate and placed on a rotating shaker for 60 minutes. The solution was centrifuged at 12,000 g for 5 minutes to allow the separation of the two immiscible phases. ASX was recovered from the upper phase, diluted opportunely and quantified by spectrophotometry as described above. The efficiency of encapsulation was estimated by the subsequent formula:

$$EE \% = \frac{ASXf}{ASXi} \times 100$$

Where ASXi represents the initial amount of ASX loaded in the NPs, whereas ASXf refers to the amount of ASX recovered from NPs after the breakage of the protein shell via enzymatic extraction.

Surface ASX (ASXs) was calculated as follows: 0.5 ml of NPs was mixed with 1 ml of ethyl acetate for 5 minutes. After centrifugation at 12,000 g for 5 minutes the supernatant was analyzed by spectrophotometry to measure ASXs by the following formula:

$$ASXs \% = \frac{ASXs}{ASXi} \times 100$$

### ABTS Radical Scavenging Activity (RSA)

For ABTS assay the procedure described with some modifications was followed. Two stock solutions, i.e. 7.4 mM ABTS and 2.6 mM potassium persulfate were prepared. The working solution was then obtained by mixing the two stock solutions in equal quantities and allowing them to react for 12 h at room temperature in the dark. The solution was then diluted opportunely with methanol (for the H.p. oleoresin), or PBS (for ASX NPs) to obtain an absorbance of 0.75 units at 734 nm. Fresh ABTS solution was prepared for each assay. Samples (20 μl) were loaded in a 96-well plate and left to react with 180 μl of the ABTS solution for 2 h in a dark condition. Then the absorbance was taken at 734 nm using a microplate reader (Bio-Tek). Results were presented as % scavenging activity following the equation:

$$\% RSA = \frac{A blank - A sample}{A blank} \times 100$$

Where A blank is the absorbance given by the solvent at 734 nm while A sample is the absorbance given by the sample.

### Chemical Stability

The chemical stability of ASX NPs was tested in solutions with different pH values (1-10) adjusted using NaOH or HCl solutions. The stability was evaluated spectrophotochemically using a UV/VIS spectrophotometer (Unicam UV2) reading the absorbance at 650 nm as a turbidimetry index.

### Effect of pH

The stability of ASX NPs was tested in solutions with different pH values (1-10) adjusted using NaOH or HCl. The stability was evaluated by spectrophotometry reading the absorbance at 660 nm as a turbidimetric index.

### UV Irradiation

The stability against UV-B light of ASX NPs and H.p. oleoresin (solubilized in DMSO as described above) was studied using a trans-illuminator (Bio-Rad, Hercules, Calif., USA). During the exposure aliquots of the samples were taken at different time points: 5′, 30′, 60′ and 120′. The % of residual ASX was determined by spectrophotometry as previously described.

### Oxidation Stability

Stability of ASX NPs and ASX from H.p. extract against chemical oxidation was analyzed according to the method proposed by Pan and co-workers with some modifications. Briefly, the H.p. oleoresin was solubilized in DMSO and diluted in water to reach a final concentration of 7.5 μg/ml. The samples 980 μl were mixed with 10 μl of a FeCl₃ solution (350 μg/ml) and incubated at room temperature for 24 hours. At different time points, an aliquot of the solution was analyzed and the % of ASX retained evaluated. The % of residual ASX from NPs was analyzed through enzymatic digestion with trypsin as previously described. The sample containing H.p. oleoresin was directly extracted in a double volume of ethyl acetate. Prior the extraction a solution of ascorbic acid (10 μl from a 1.3 mg/ml stock solution) was added to the sample in order to stop the oxidative reaction. The % of ASX retained was evaluated using the following equation:

$$ASX \% = \frac{ASXr}{ASXi} \times 100$$

where ASXr corresponds to the amount of ASX retained after the exposure to the oxidation condition and ASXi is the initial amount of ASX determined through enzymatic extraction after encapsulation process.

### Storage Stability

Storage stability of ASX NPs and H.p. oleoresin was evaluated by an accelerated system at at 65° C. The samples were kept in a static oven (Memmert, Germany) in closed polypropylene tubes. To study the stability of the extract, ethyl acetate was removed to obtain only the oleoresin from H.p. At different time points the samples were analyzed for the content of residual ASX by spectrophotometry and an HPLC analyses were performed.

### Simulated In Vitro Digestion

In vitro digestion was performed following Infogest protocol (Minekus et al. 2014). Simulated gastric fluids (SGF) and Simulated Intestinal Fluid (SIF) were prepared following the above mentioned protocol. The duration of the two phases were 1 h for the gastric and 4 h for the intestinal phase. The enzyme used were pepsin and pancreatin for the gastric phase (pH 3) and the intestinal phase (pH 7) respectively. Bile salt in the form of sodium cholate was added to SIF to a final concentration of 10 mM. Briefly, 0.5 mL samples dispersed in water and SGF were mixed in a ratio 1:1, the pH was adjusted to 3 with HCl 1M. After 1 hour, SIF was added in a ratio 1:1 and the pH adjusted to 7 with NaOH 1M. The reaction was conducted at 37° C. in a rotating shaker. At different time points samples were extracted with a double volume of ethyl acetate. The amount of ASX released from ASX NPs were evaluated by spectrophotometry. De-esterification degree was evaluated through HPLC analysis.

### Statistical Analysis

All measurements were performed at least three times and the results were reported as mean value±standard deviation. Statistical analysis was performed using one-way analysis of variance (ANOVA) using SigmaPlot v12.5 software (Systat Software Inc. CA, US).

### Cell Culture

The experiments dealing with cells have been carried out in collaboration with the laboratory of animal cell cultures of the University of Verona directed by Prof. Roberto Chignola. The utilized cell lines were from ATCC (American Type Culture Collection Rockville, Md., USA).

Monocyite macrophages cell line (J774A) from adult mice, HepG2 (human hepatocellular carcinoma, ATCC HB-8065, cell type: epithelial) and Caco2 (human colorectal adenocarcinoma, ATCC HT-B37, cell type: epithelial), were cultured in RPMI 1640 medium (Biochrom AG, Berlin, Germany) supplemented with 10% fetal bovine serum (FBS), 2 mM glutamine (Sigma, St. Louis, Mo., USA) and 35 mg/l of gentamycin. Cells were grown at 37° C. in a humidified 5% CO₂ atmosphere in T75 culture flasks (Greiner Bio-One, Frickenhausen, Germany) and periodically were diluted with fresh medium to avoid starvation. The concentration of viable cell was measured by an haemocytometer in presence of trypan blue (0.1% in PBS, Biochrom AG).

### Cytotoxicity Assay

HepG2 cells were initially seeded in 96-wells culture plates at a density of 25000 cells in 200 μl of complete medium. After 6 h, 25 μl of medium was removed and substituted with a same volume of antioxidant, and incubated for 30 minutes at 37° C. The antioxidant was represented by H.p. oleoresin dissolved in 1% DMSO and ASX NPs both tested at the following concentrations: 0.2, 0.1 and 0.05 mg/ml. Methanol 20% was used as negative control, RPMI medium as positive control and DMSO 1% to test if even in small quantity it could affect cell vitality. Cells were counted using light phase-contrast microscopy (Olympus IX51; Olympus, Tokyo, Japan), using a Burker chamber, and the vital dye trypan blue (dilution 1:2 v/v) to exclude dead cells. ATP was determined by the luciferine/luciferase method using CellTiter-Glo® Luminescent Cell Viability Assay Kit (Promega, Madison, Wis., USA), following the manufacturer's instructions. Emitted light was measured using a microplate luminometer (FLₓ 800, Bio-tek Instruments) and data were expressed in luminescence arbitrary units.

### Cellular Antioxidant Activity by Flow Cytometry

Adult mice macrophages cell line J774A were seeded at a density of 1×10⁵ cells in 2 ml of RPMI medium. After an incubation of 24 h at 37° C. 1 ml of the medium was removed and 500 μl of DCFH-DA 50 μM and 500 μl of antioxidant were added. The tested antioxidants were: H.p. oleoresin dissolved in 1% DMSO and ASX NPs dissolved in RPMI both at the concentration of 14, 7, 3.5, 1.75, 0.875 and 0.438 μg/ml of H.p. oleoresin. As a control, also the antioxidant activity of native WPC protein was analyzed at concentrations of 25, 12.5 and 6.25 μg/ml. After incubation with the antioxidant, the medium was removed and a same volume (500 μl) of an oxidant species was added to the plate (600 μM ABAP and 0.002% H₂O₂ were tested as different stressing conditions). The treated cells were incubated for 30 minutes at 37° C. After incubation the cells were scraped from the plate to obtain a cellular suspension for the cytofluorimetric analysis. The suspension was kept at 4° C. until use. The cells associated fluorescence was measured using a Guava easyCyte™ 5 flow cytometer v.2.7 software (Merck Millipore, Billerica, Mass., U.S). The cytometer was equipped with 488 nm, 20 mW, blue laser light, and forward scatter (FSC) photodiode and side scatter (SSC) photomultiplier. Green fluorescence 525/30 filter, yellow 583/26 and red 680/30 filters allow analysis of fluorescence emissions from samples. Calibration of the cytometer was routinely checked using the Guava EasyCheck kit (Merck Millipore, Billerica, Mass., U.S.) according to the manufacturer's instructions. The raw data were exported, and then processed and analyzed by Mathematica software.

### Confocal Microscopy

The uptake of ASX NPs was analyzed by confocal microscopy. To this purpose, ASX NPs were labelled with fluorescein isothiocyanate (FITC). In particular, 10 mg of lyophilized ASX NPs were resuspended in 1.695 μl of carbonate buffer pH 7.3. Ten milligrams of FITC was resuspended in 1 ml of DMSO. Eighty μl of FITC concentrated solution was added to the ASX NPs solution and left to react for 2 h in the dark. The non-reacted FITC was removed using a PD Mini-Trap desalting column with Sephadex G-25 resin (Supelco, Bellefonte, Pa., U.S.).

The following cell lines were used: Caco2 (human colorectal adenocarcinoma, ATCC HT-B37, cell type: epithelial), HepG2 (human hepatocellular carcinoma, ATCC HB-8065, cell type: epithelial). Cells were routinely cultured at 37° C. in a humidified 5% CO2 atmosphere, in RPMI 1640 (Biochrom AG, Berlin, Germany) supplemented with 2 mM glutamine (Sigma-Aldrich, St. Louis, Mo., USA), 35 mg/l gentamycin (Biochrom AG) and 10% heat-inactivated fetal bovine serum (Biochrom AG). Cells were seeded into the wells of glass bottom μ-Slide IbiTreat chambers (Ibidi GmBH, Martinsried, Germany) at the initial cell density of 5·10⁴ cells/well in 200 μl complete growth medium and incubated for 24 hours at 37° C. A volume of 50 μl of FITC-labelled ASX NPs solution (green fluorescence) was then added, and the cells were further incubated at 37° C. for 1 hour. Cells were carefully washed with PBS, fixed with 4% (w/v) paraformaldehyde for 30 min and, after washings with PBS and quenching with 50 mM NH₄Cl, permeabilized with PBS-0.1% Triton X-100. Cells were then stained with rhodamine-phalloidin (to label F-actin, red fluorescence; Cytoskeleton, Denver, Colo., USA) for 30 min and then with DAPI (to label nuclei, blue fluorescence; Sigma-Aldrich, St. Louis, Mo., USA) for 10 min. Images at Δz=0.5 μm were collected using the SP5 confocal microscope from Leica Microsystems (Mannheim, Germany) with 63× objective (HCX PL APO λ blue 1.4NA OIL). Image analyses were performed with ImageJ v. 2.0.0 software.

### Cell Nanoparticles Uptake

To gain information on the mechanism involved in the cellular uptake, the experiment was conducted in the presence of a blocking condition, i.e. at low temperature. Indeed, Caco2 and HepG2 cells were incubated with FITC-labelled ASX NPs (0.02 mg/ml) at 4° C. for 30 minutes and at 37° C. as a control.

### Example 1

### Physical Evaluation of Astaxanthin WPC NPs

ASX was extracted from H.p. cells with a maximum yield of 1.1% w/w. HPLC analysis of the oleoresin was performed to understand if the microwave-assisted extraction and the thermal treatment at 50° C. could have detrimental effects on ASX integrity. The profile showed the presence of ASX mainly in the mono-esterified form (80%), followed by the di-esterified form (18%) and a low amount of free form (2%). Traces of other carotenoids were observed but not quantified.

WPC was employed as a biocompatible matrix to improve the bioavailability and dispersibility of ASX in water through emulsification-solvent evaporation technique. The process was optimized taking into account two parameters.

The first parameter considered was WPC concentration, because as reported earlier the amount of the matrix material can influence not only the dimension of the NPs, because of the layering effect of the proteins that tend to form growing structures until a stable conformation is reach, but also the time requested to release the bioactive compound during digestion process.

The solution containing ASX NPs was transparent with a red-orange bright color, but the increasing concentration of protein leads to a loss of transparency. The average size, PDI and zeta potential were considered as reference parameters to evaluate the process. As shown in Table 1, the increase of proteins amount lead to a growth of the dimensions of ASX NPs from an average size of 90 nm, obtained with the lowest concentration, to 128 nm of the 10% WPC. The data show an evident dependence of the dimensions from protein concentration. PDI values were low for all the formulations, ranging from 0.247 of the 1% formulations to 0.261 of the 10% ones. Zeta potential values were highly negative, due to the WPC shell that at neutral pH are negatively charged.

TABLE 1
Z average, (b) PDI, and (c) Zeta potential variation as a consequence
of the different proteins concentration used to produce ASX NPs

| | | | Zeta |
|---|---|---|---|
| Proteins concentration | Z average | | Potential ± |
| % | (nm) | PDI | Zeta dev. |
| 0.1 | 90.83 ± 2.02 | 0.247 ± 0.0015 | −31.3 ± 8.95 |
| 0.5 | 92.3 ± 2.34 | 0.259 ± 0.005 | −31.2 ± 7.7 |
| 0.8 | 95.67 ± 0.08 | 0.262 ± 0.010 | −29.8 ± 6.87 |
| 1 | 103.37 ± 0.98 | 0.255 ± 0.014 | −28.5 ± 6.5 |
| 2 | 106.1 ± 1.25 | 0.260 ± 0.011 | −24.7 ± 6.32 |
| 5 | 116.5 ± 0.36 | 0.254 ± 0.005 | −20.0 ± 5.47 |
| 8 | 125.07 ± 1.11 | 0.259 ± 0.023 | −17.9 ± 4.56 |
| 10 | 128.27 ± 1.98 | 0.261 ± 0.011 | −17.0 ± 4 |

In was observed a decrease of zeta potential value by increasing the % of WPC used for encapsulation: 10% preparation showed a zeta potential of −17.0±4. The although the best values of zeta average, PDI and zeta potential were obtained with WPC concentration ranging from 0.1 to 1%, a rapid degradation of ASX was experienced at WPC concentrations from 0.1% to 0.5%. As a consequence, the subsequent experiments were performed using a WPC concentration of 1%.

The second parameter considered was the amount of H.p. oleoresin used. As shown in Table 2, the diameter of ASX NPs decreases with the increase of oleoresin, with a minimum of 94.98±1.27 and with a PDI of 0.235±0.015. This sample showed a surface charge higher than −20 mV (−17.9±4.56 mV), underlining the instability of the structure. This is possibly due to the high amount of oleoresin exceeding the amount of proteins constituting the shell. Satisfactory results were given by the NPs made with 4.5 mg of H.p. oleoresin with a Z-average of 102.7±0.36, PDI of 0.242±0.016 and high negatively surface charge of −28.5±6.5.

TABLE 2
Z average, PDI, and Zeta potential variation as a consequence of
the different oleoresin concentrations used to produce ASX NPs.

| | | | Zeta |
|---|---|---|---|
| | Z average | | Potential ± |
| % H.p. oleoresin | (nm) | PDI | Zeta dev. |
| 1 | 117.40 ± 0.52 | 0.256 ± 0.008 | −31.3 ± 8.95 |
| 2.5 | 108.53 ± 3.16 | 0.272 ± 0.012 | −31.2 ± 7.7 |
| 3.5 | 102.87 ± 1.97 | 0.245 ± 0.009 | −29.8 ± 6.87 |
| 4.5 | 102.7 ± 0.36 | 0.242 ± 0.016 | −28.5 ± 6.5 |
| 6.5 | 98.09 ± 1.71 | 0.245 ± 0.057 | −24.7 ± 6.32 |
| 9 | 96.99 ± 0.62 | 0.248 ± 0.032 | −20.0 ± 5.47 |
| 11 | 94.98 ± 1.27 | 0.235 ± 0.015 | −17.9 ± 4.56 |

The encapsulation efficiency was 96±2.5%. The minor loss of ASX could be caused by the oxidation generated from the sonication process, or from the incomplete degradation of the protein shell during the enzymatic extraction. HPLC analysis of the extract from ASX NPs was performed and compared to the ones of the H.p. oleoresin before the encapsulation process, no modifications were observed (data not shown). The payload of the higher concentration reached was 11%.

### Example 2

### Astaxanthin-WPC NPs with Chitosan

Evaluation of ASX NPs with WPC 1% and Chit 0.1-0.05% was done.

The superficial charge of ASX NPs was negative, since chitosan is a cationic polymer it was decided to try to form another shell around the already formed NP.

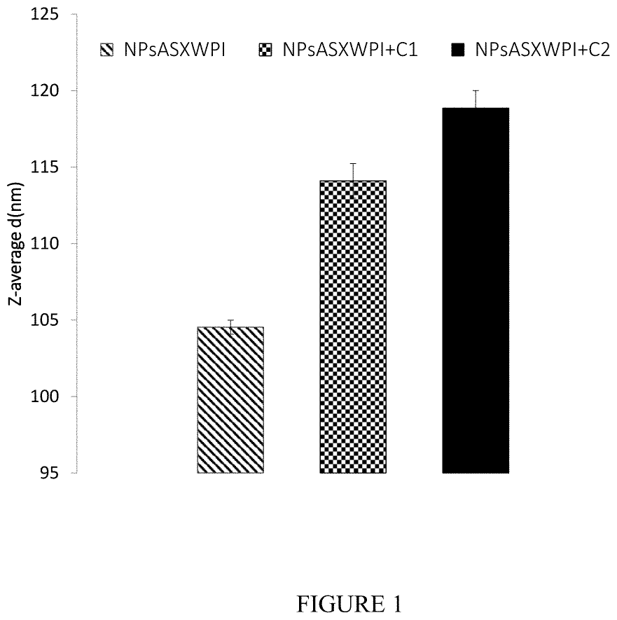

Size: NPs dimension increase with the addiction of chitosan in a dependent way from the concentration (from 105 to 120 nm) (FIG. 1).

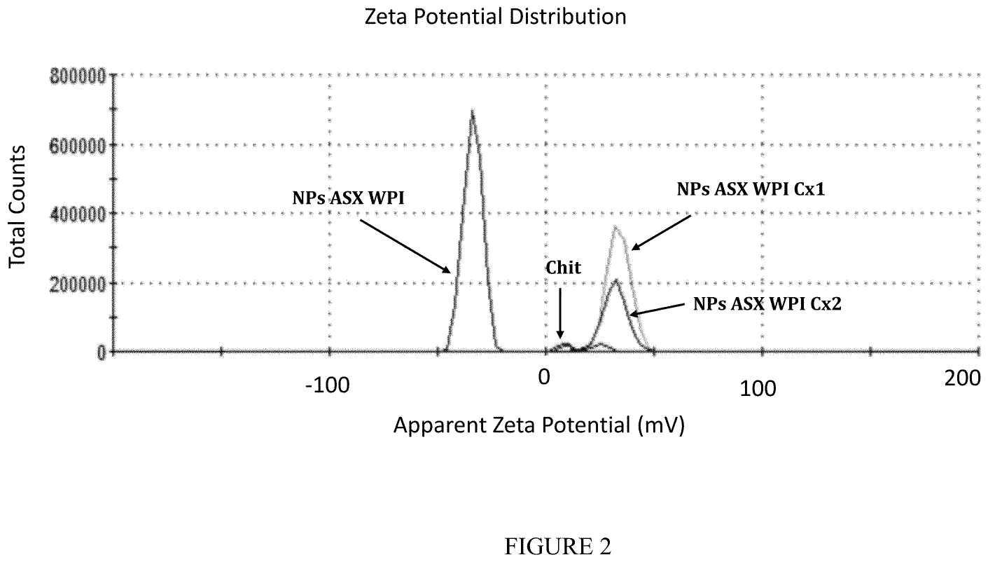

Superficial charge: starting charge of the ASX NPs was negative (−33±6.63 mV) but after the addition of chitosan became positive 33.5±5.21 (FIG. 2).

### Example 3

### Spray Dry of ASX NPs

Different formulations and conditions of encapsulation were evaluated and are present in Table 3.

Different polymers were tested to obtain ASX particles powder through spray drying. Whey protein isolate (WPI) was used in all the formulation to produce the main shell. As filler, two modified starches were studied: CAPSUL and HICAP, frequently used for spray dry applications. Non-modified starch was analyzed due to higher compliance showed by consumers for natural ingredients. Maltodextrin (MD) and gum arabic are the most employed polymer for spray dry with optimal encapsulation and specific release features.

Some of matrices were added during the sonication process with WPI, other added after sonication or at the end of the evaporation process to study the influence of their presence in the NPs formation. Formulations nos. 2,5,8,9, and 10 gave satisfactory results from the point of view of dispersibility in water, PDI and average size of the particles when dispersed in water. The best result was showed by the formulation no. 10 showing a PDI of 0.2 and an average size of 147.2 nm.

TABLE 3
Composition of formulations expressed as % w/v of different
polymers to obtain the spray dry powder of astaxanthin.

| | | Modified | Modified | Whey | | | |
|---|---|---|---|---|---|---|---|
| | | starch | starch | protein | Gum | | |
| | Starch | (HICAP) | (CAPSUL) | isolate | Arabic | Maltodextrin | |
| Formulations | % | % | % | % | % | DE 19% | Note |
| Formulation | 0 | 0 | 0 | 10 | 5 | 0 | Emulsification |
| 1 | | | | | | | together |
| Formulation | 0 | 0 | 0 | 20 | 5 | 0 | GA added |
| 2 | | | | | | | after |
| | | | | | | | emulsification |
| Formulation | 19 | 0 | 0 | 0 | 5 | 0 | Gelatinized |
| 3 | | | | | | | starch |
| | | | | | | | Emulsification |
| | | | | | | | together |
| Formulation | 0 | 0 | 0 | 10 | 5 | 0 | GA added |
| 4 | | | | | | | after |
| | | | | | | | emulsification |
| Formulation | 0 | 19 | 0 | 1 | 5 | 0 | GA added |
| 5 | | | | | | | after |
| | | | | | | | emulsification |
| Formulation | 0 | 0 | 0 | 5 | 10 | 0 | GA added |
| 6 | | | | | | | after |
| | | | | | | | emulsification |
| Formulation | 0 | 0 | 0 | 5 | 10 | 0 | Emulsification |
| 7 | | | | | | | together |
| Formulation | 10 | 0 | 0 | 5 | 0 | 0 | Emulsification |
| 8 | | | | | | | together |
| Formulation | 0 | 0 | 19 | 1 | 0 | 0 | Emulsification |
| 9 | | | | | | | together |
| Formulation | 0 | 0 | 0 | 10 | 0 | 5 | MD added |
| 10 | | | | | | | after |
| | | | | | | | emulsification |

TABLE 4
Characteristics of the different powder formulations

| | Solubility | |
|---|---|---|
| Formulation | description | DLS data |
| 1 | Not easy to | PDI too high |
| | disperse | |
| 2 | Easy | PDI 0.3 |
| | dispersible | D(nm) 226 nm |
| 3 | Not | PDI too high |
| | dispersible | |
| 4 | Easy | PDI too high |
| | dispersible | |
| 5 | Easy | PDI 0.36 |
| | dispersible | D(nm) 218 nm |
| | and clear | |
| | solution | |
| 6 | Easy | PDI too high |
| | dispersible | |
| 7 | Easy | PDI too high |
| | dispersible | |
| 8 | Not easy | PDI 0.35 |
| | dispersible | D(nm) 140 nm |
| | | Pellet formation |
| 9 | Easy | PDI 0.5 |
| | dispersible | D(nm) 786 nm |
| 10 | Easy | PDI 0.2 |
| | dispersible | D(nm) 147.2 nm |

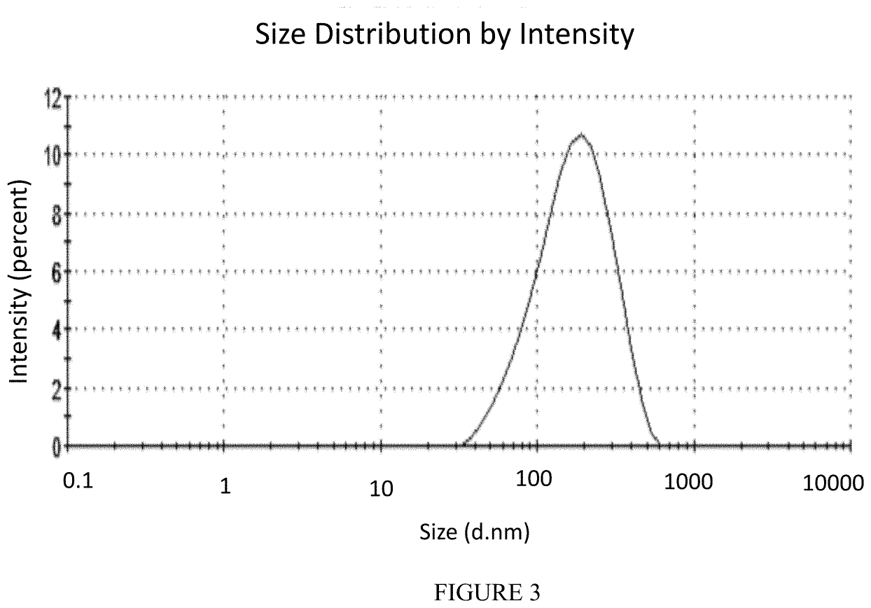

FIG. 3 shows the size distribution of particles obtained with Formulation 10.

### Example 4

### Astaxanthin-WPI-MD Powder Characterization

Astaxanthin water dispersible powder (ASX WD P) was produced via spray drying.

Different combinations of polymers were evaluated in order to obtain ASX particles with diameter around 100-200 nm when re-dispersed in water. Among the different combinations a mixture of WPI and MD was chosen. The obtained powder was bright orange in color and easily dispersible in water. The encapsulation efficiency (EE) was 95.73% and the yield of encapsulation (YE) 85±2%. ASX concentration was 11.8 mg/g of powder and antioxidant activity was 5 times higher than Trolox, 26 times higher than β-carotene and 8 higher than the ASX oleoresin at the same concentration.

### Example 5

### Astaxanthin-WPI-MD Powder Characterization

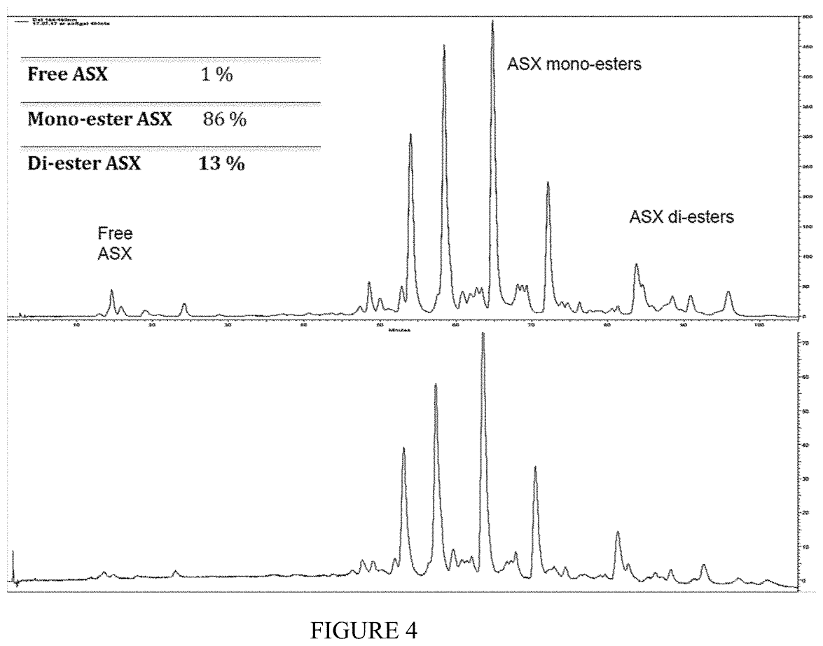

ASX extracted from the powder was composed mainly by mono-esters of ASX (86%), followed by the di-esters (13%) and 1% of the free form, the same as the initial oleoresin (FIG. 4).

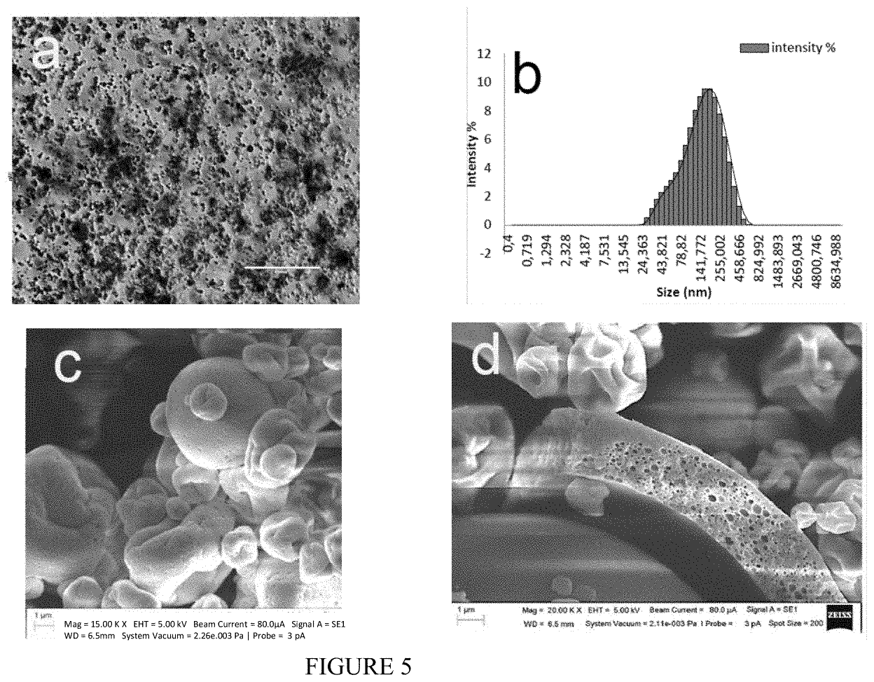

The particle size was found to be in the range of 100-200 nm, with a polydispersity index PDI of 0.214. Particles morphology was evaluated by optical microscope and SEM (FIG. 5), both images confirm the presence of spherical particles.

### Example 6

### Comparison of Release Profile During In Vitro Digestion

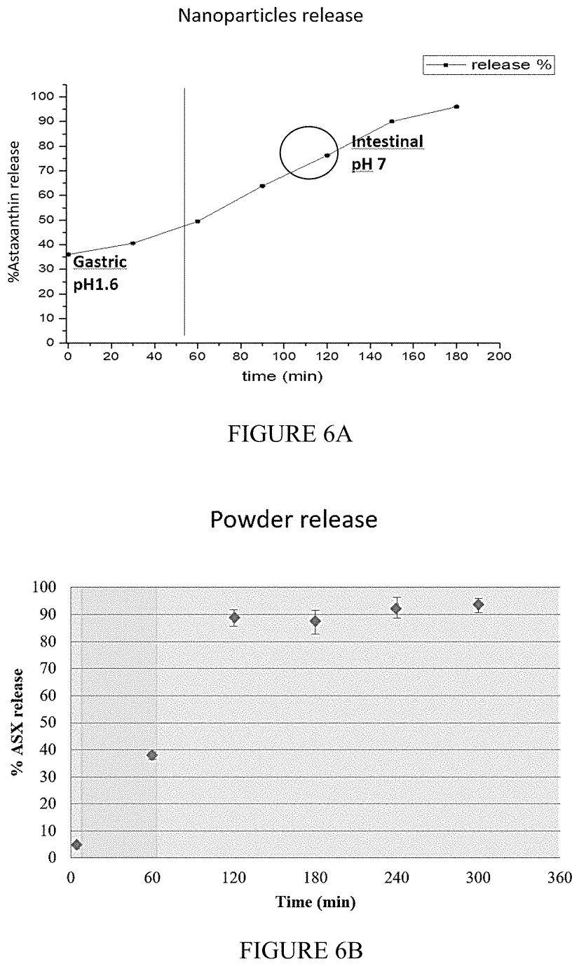

The release profile of ASX in the form of NPs and in the form of powder are shown in FIGS. 6A-B.

The absorption of carotenoids during digestion occurs mainly in the first part of intestine, where the uptake through the intestinal mucosa takes place. It is also acknowledged that the highly acidic conditions typically present in the stomach can significantly affect the stability of ASX.

The ideal system should protect ASX during the transit through the stomach and allow the release of ASX within the first two hours of intestinal stage. ASX NPs release during gastric phase was about 45% (FIG. 6A). This release can be explained considering that during this phase an evident aggregation of the NPs was observable probably as a consequence of the low pH value close to the pI of the whey proteins. In the intestinal phase the aggregates disappeared. The release of ASX from the NPs reached about 65% after 2 hours of intestinal digestion, that, in this model, represents the small intestine. After 4 hours of intestinal digestion the amount of ASX released was about 90%.

In accordance with the results obtained for the ASX NPs, the ASX particles powder formulation (FIG. 6B) showed a release of 38% at the end of gastric stage, during the intestinal phase the release of the bioactive compound rapidly increased until reaching 93% after 4 hours of the intestinal phase. The similar release profile showed that the higher solid content of the formulation does not affect the release of ASX during in vitro simulated digestion.

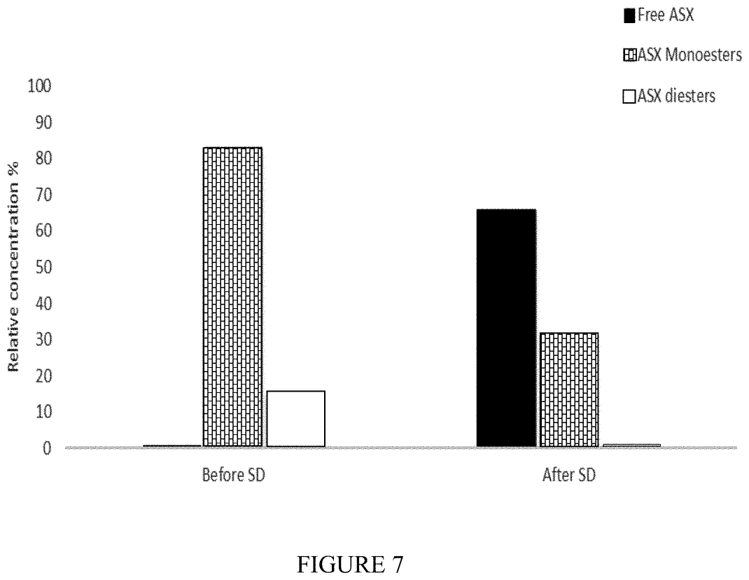

Simulated digestion (SD) was performed not only to address the bioaccessibility of the encapsulated ASX but also to evaluate possible chemical modifications of the carotenoid. HPLC analysis of the extracted from NPs collected before and after digestion could give a picture of the esterification degree of ASX. FIG. 7 shows that the composition of the extract from the NPs before digestion was mainly represented by monoesters and diesters accounting on the whole for the 99% of the ASX present, while after two hours of the intestinal digestion, the major form was represented by free ASX (66%). After 4 hours of digestion the conversion to the free form reached 75%.

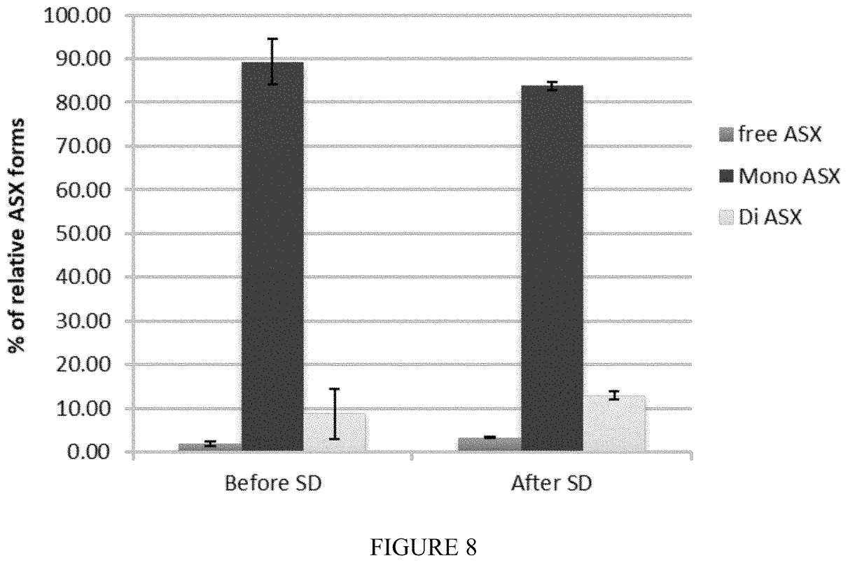

FIG. 8 shows the composition of ASX esters in the ASX particles powder before and after digestion.

HPLC analysis of the H.p. extract released during simulated digestion in vitro showed a high rate of de-esterification of ASX at intestinal level from 0.88 to 75.36% of free astaxanthin (FIG. 7). This parameter is considered as an index of the theoretical bioavailability, defined as the rate and extent to which the bioactive compound or a drug is absorbed and becomes available at the site of action making the de-esterification of carotenoids a mandatory step for the absorption through the intestinal mucosa. For the water dispersible powder form the rate was extensively lower (FIG. 8), from 1.8 to 3.4% of free ASX detected at the end of the intestinal stage. A possibility for the low de-esterification rate could be the higher amount of ASX in the formulation that needs to be processed by lipase. Indeed, the amount of ASX and lipid present in the oleoresin used in this formulation is 27.5 times higher than the one used to produce the NPs formulation, and the simulated digestion protocol employed is designed mostly for the digestion of proteins.

### Example 7

### Endocytosis of NPs: Confocal Microscopy Analyses

The following cell lines were used: Caco2 (human colorectal adenocarcinoma, ATCC HT-B37, cell type: epithelial), HepG2 (human hepatocellular carcinoma, ATCC HB-8065, cell type: epithelial), J774A1 (mouse reticulum cell sarcoma, ATCC TIB-67, cell type: monocyte/macrophage). Cells were routinely cultured at 37° C. in a humidified 5% CO2 atmosphere, in RPMI 1640 (Biochrom AG, Berlin, Germany) supplemented with 2 mM glutamine (Sigma-Aldrich, St. Louis, Mo., USA), 35 mg/l gentamycin (Biochrom AG) and 10% heat-inactivated foetal bovine serum (Biochrom AG). Cells were seeded into the wells of glass bottom μ-Slide IbiTreat chambers (Ibidi GmBH, Martinsried, Germany) at the initial cell density of 5·10⁴ cells/well in 200 μl complete growth medium and incubated for 24 hours at 37° C. A volume of 50 μl of FITC-labelled NPs solution (green fluorescence) was then added, and the cells were further incubated at 37° C. for 1 hour. Cells were carefully washed with PBS, fixed with 4% (w/v) paraformaldehyde for 30 min and, after washings with PBS and quenching with 50 mM NH₄Cl, permeabilized with PBS-0.1% Triton X-100. Cells were then stained with rhodamine-phalloidin (to label F-actin, red fluorescence; Cytoskeleton, Denver, Colo., USA) for 30 min and then with DAPI (to label nuclei, blue fluorescence; Sigma-Aldrich, St. Louis, Mo., USA) for 10 min. Images at Δz=0.5 μm were collected using the SP5 confocal microscope from Leica Microsystems (Mannheim, Germany) with 63× objective (HCX PL APO λ blue 1.4NA OIL). Image analyses were performed with ImageJ v. 2.0.0 software.

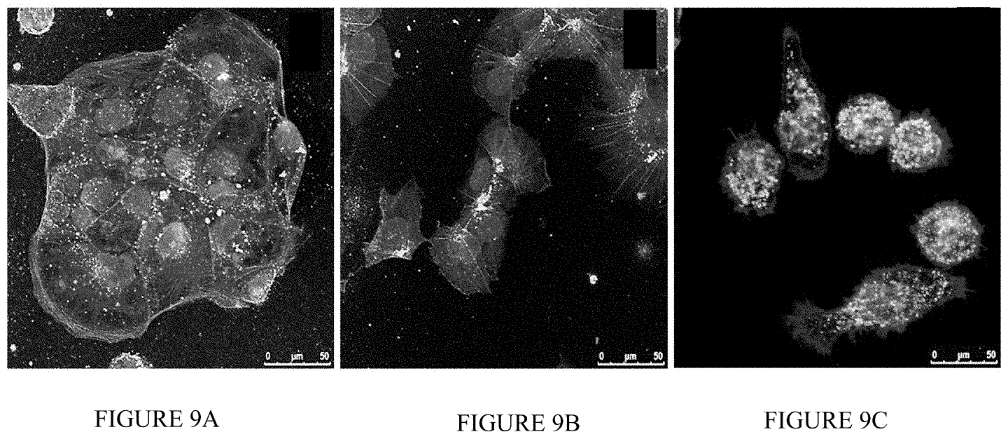

In FIGS. 9A-C confocal microscopy images are depicted, showing the cell uptake of the ASX NPs.

### Example 8

### Production of ASX Particles in Agglomerates Form

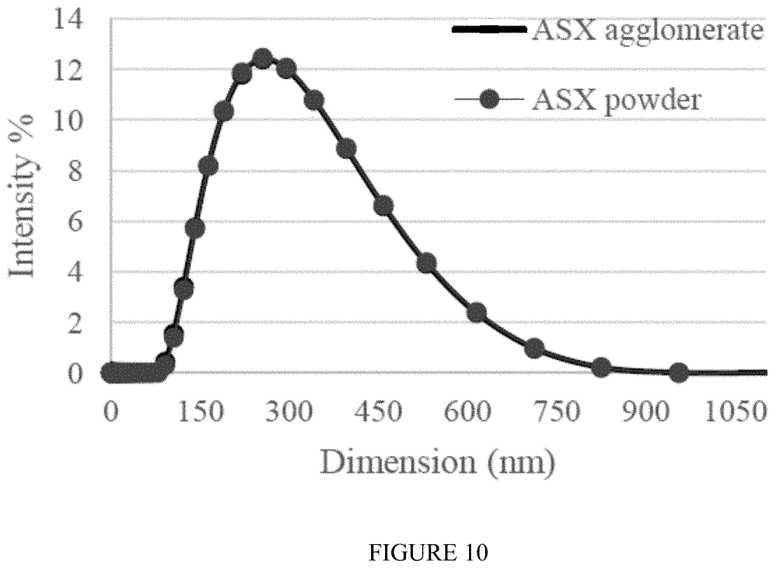

The results of the size distribution analysis of the agglomerated obtained by fluid bed in comparison with the water dispersible ASX particles powder formulation are shown in FIG. 10. Upon resuspension the average diameter of the NPs liberated are almost identical.

### Example 9

### Production of Curcumin-WPC/WPI NPs

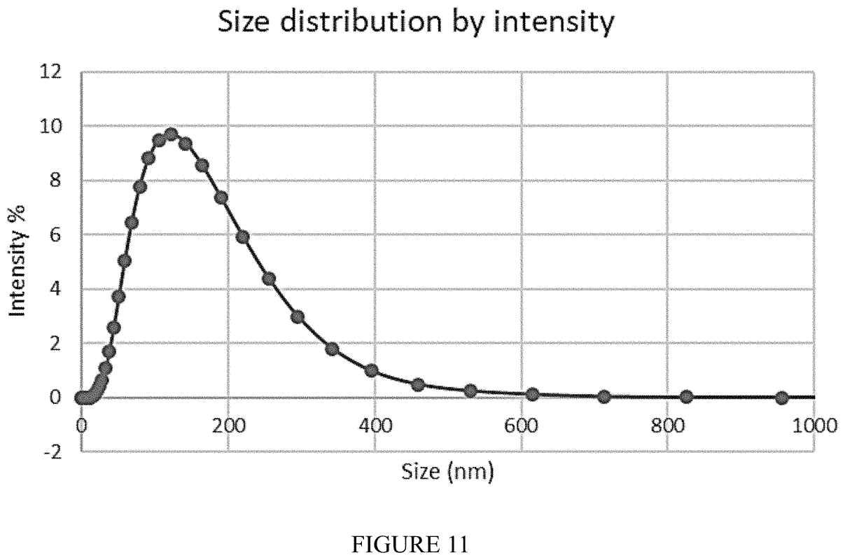

Curcumin from Curcuma longa (turmeric) was encapsulated by the same procedure disclosed above. Briefly, WPC or WPI was dissolved in distilled water in a concentration range between 1 and 10%. The solution was stirred for 30 minutes at room temperature without pH modification. Curcumin was dissolved in ethyl acetate and combined with the protein solution at a ratio of 9:1 (protein solution:extract). A fine emulsion was produced using an ultra-sonicator for 10 minutes at a potency of 10 W (Microson ultrasonic cell disruptor XL). At the end of the process ethyl acetate was removed using a nitrogen flow in the dark. The WPC or WPI curcumin-NPs were kept in the dark at 4° C. until use. Further layers enveloping the NPs can be produced as described above. Water dispersible powders can be obtained with the same procedure described for ASX-reach oleoresin. FIG. 11 shows the DLS analysis of the NPs obtained.

### Example 10

### Production of Omega 3-WPC/WPI NPs

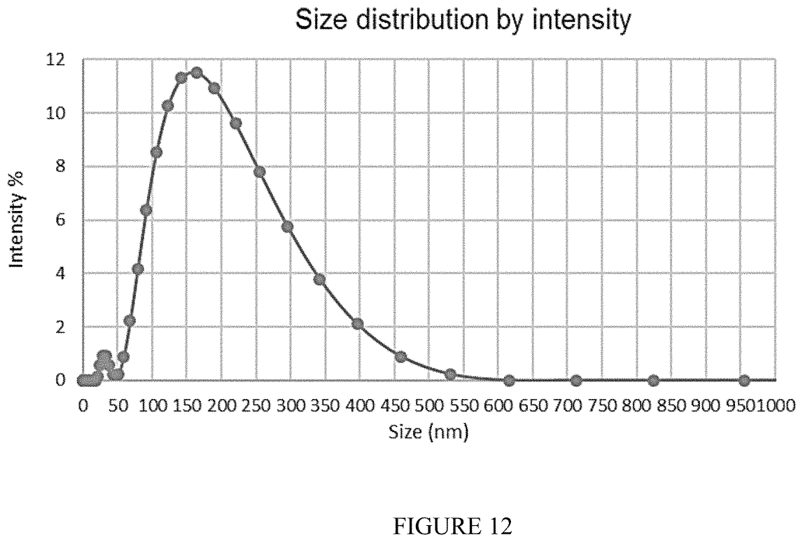

Fish oil rich in long chain omega 3 fatty acid such as docosahexaenoic acid (DHA) and eicosapentaenoic acid (EPA) was encapsulated by the same procedure disclosed above. Other sources of omega 3 fatty acid like cod liver oil or flaxseed oil are also suitable. Briefly, WPC or WPI was dissolved in distilled water in a concentration range between 1 and 10%. The solution was stirred for 30 minutes at room temperature without pH modification. Fish oil was dissolved in ethyl acetate and combined with the protein solution at a ratio of 9:1 (protein solution:extract). A fine emulsion was produced using an ultra-sonicator for 10 minutes at a potency of 10 W (Microson ultrasonic cell disruptor XL). At the end of the process ethyl acetate was removed using a nitrogen flow in the dark. The WPC or WPI fish oil-NPs were kept in the dark at 4° C. until use. Further layers enveloping the NPs can be produced as described above. Water dispersible powders can be obtained with the same procedure described for ASX-reach oleoresin. FIG. 12 shows the DLS analysis of the NPs obtained.

### Example 11

### Encapsulation of Oleoresin

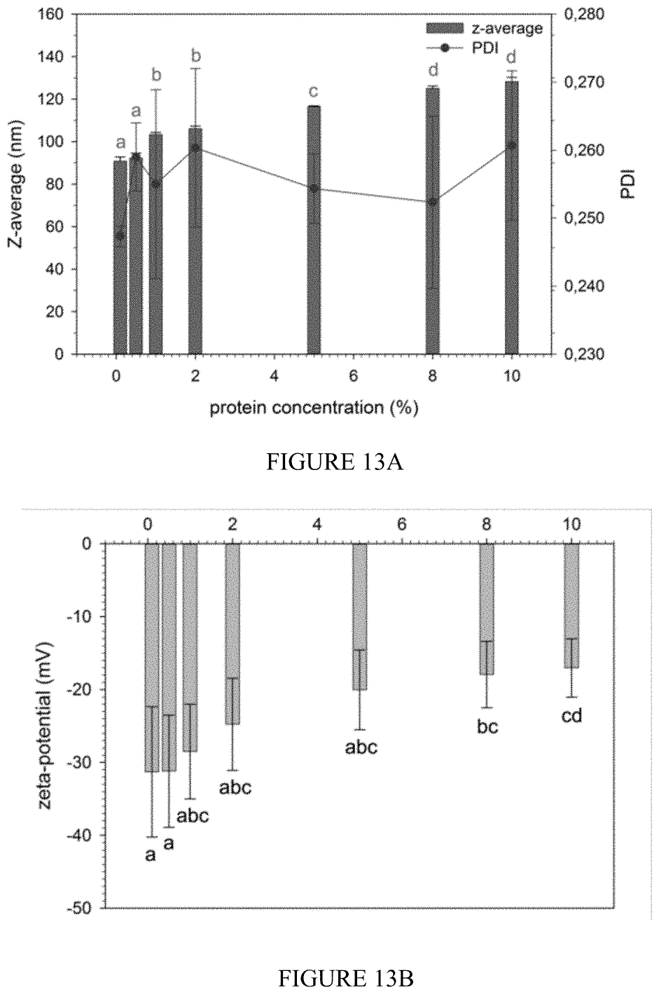

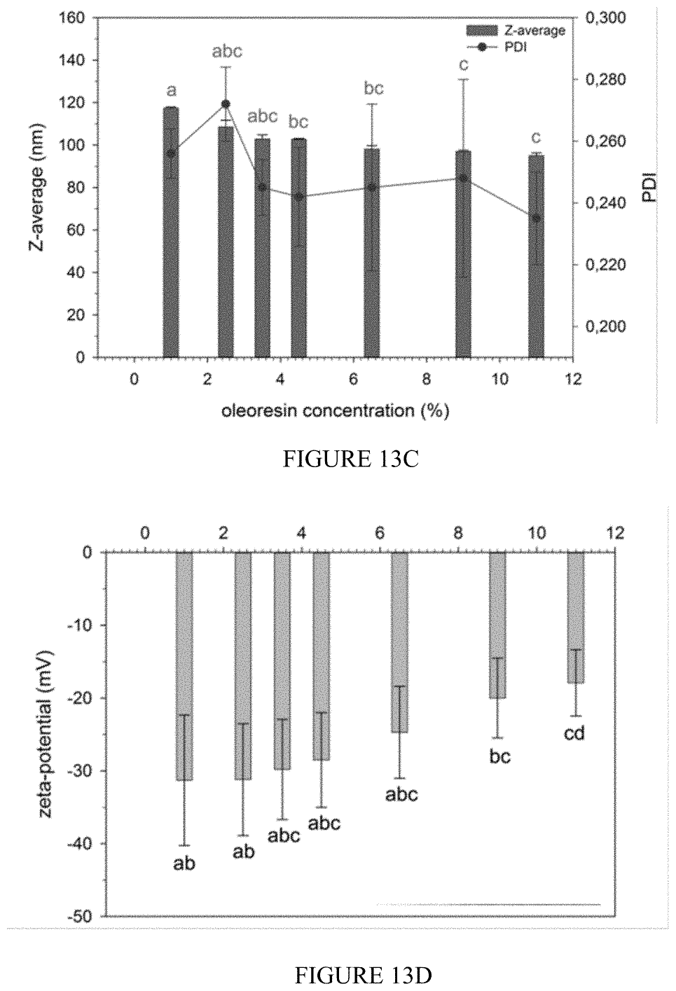

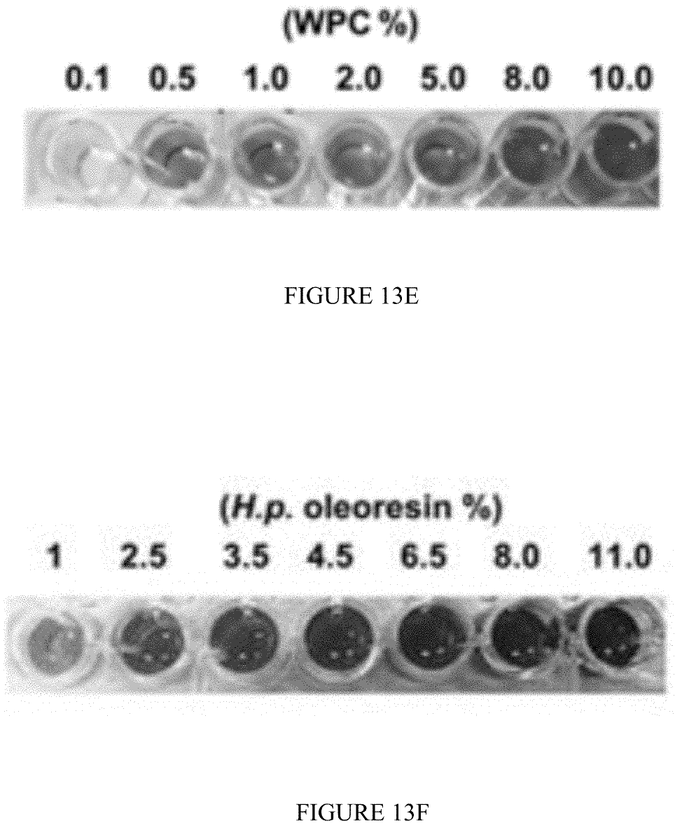

The encapsulation of H.p. oleoresin was carried out by emulsification-solvent evaporation approach, in which the lipophilic material is solubilized in ethyl acetate end emulsified with the water phase containing WPC that acts as a stabilizer of oil-in-water interfaces. Two key parameters were evaluated to optimize the process. The first was the concentration of WPC. Size (Z-average), polydispersity index (PDI) and charge (zeta-potential) were monitored to study the role of protein concentration on the formation of the nanoparticles. As shown in FIG. 13A, keeping the oleoresin concentration at 1%, the increase of proteins concentration led to the increase of the NPs diameter from an average size of 90 nm, obtained with the lowest concentration, to 128 nm with 10% WPC. PDI values were low for all the formulations, indicating a homogeneous size of NPs population, but no statistically significant differences were observed among the samples.

Zeta-potential values of the NPs were negative (FIG. 13B) due to the WP shell that at neutral pH is negatively charged. Values higher than ±20-30 mV are normally associated to stable NPs since in this potential range strong repulsive forces inhibit natural aggregation phenomena based hydrophobic and Van der Waals interactions. The observed decrease in the magnitude of the zeta-potential by increasing protein concentration can be the result of the different extent of the unfolding of the proteins at interfaces, multilayer formation, or preferential adsorption of certain proteins as previously described. Although the best values of Z-average, PDI and zeta-potential were obtained with WPC concentrations ranging from 0.1 to 1%, a rapid degradation of ASX was experienced at concentrations from 0.1% to 0.5%. As a consequence, the following experiments were performed using 1% WPC concentration.

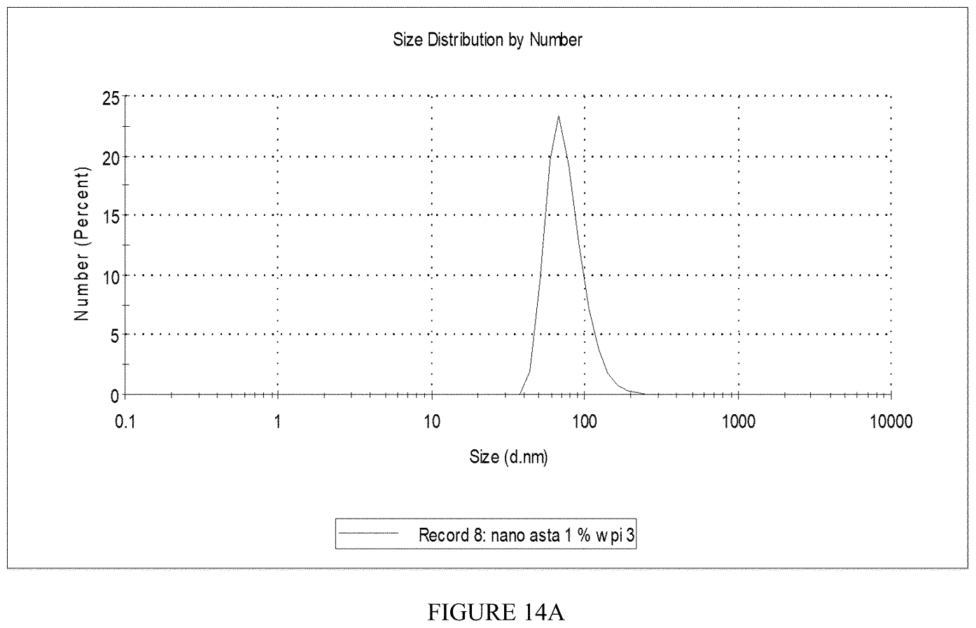

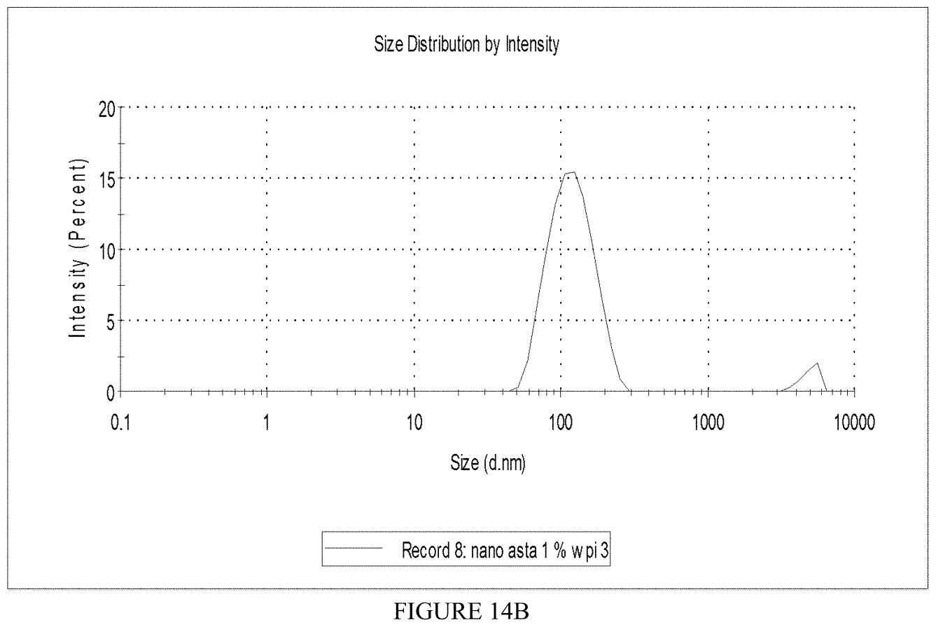

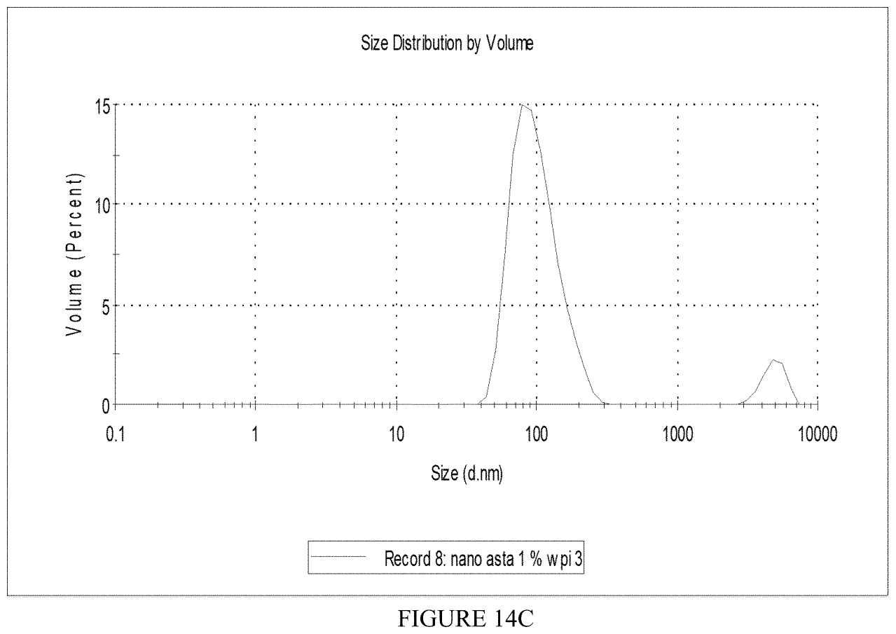

The second parameter considered was the concentration of H.p. oleoresin. As shown in FIG. 13C, the diameter of the NPs slightly diminished by increasing oleoresin concentration. This result is in agreement with the previous data shown in FIG. 13A since the increase of oleoresin-to-protein ratio (WPC was kept at 1%) would diminish the multilayer aggregation of the proteins decreasing the diameter of the particles. At the last point, obtained with 11% oleoresin, the main diameter reached 95 nm. Also in this case the zeta-potential magnitude decreased (FIG. 13D), in agreement with the reduction of the diameter, reaching a surface charge higher than −20 mV (−17.9±4.6 mV) at the last point. This value is normally associated with low stability of the nano structure. This is possibly due to the high amount of oleoresin that might have exceed the capacity of the proteins to arrange properly on oil drop surface and thus influencing the surface charge of the realized particles. Also, in this case the PDI values were low and no significant differences were measured between the samples obtained by increasing oleoresin concentration. The appearance of the nanoparticles produced as function of protein concentration and H.p. oleoresin concentration is presented in FIG. 13E and FIG. 13F respectively. Satisfactory results were given by the NPs produced with 4.5% of H.p. oleoresin with a Z-average of 103 nm, PDI of 0.242 and a surface charge of −28.5 mV. DLS results of the best solution (1% WPC, 4.5% H.p. oleoresin) are presented in FIGS. 14A-C.

### Example 12

### Characterization of NPs

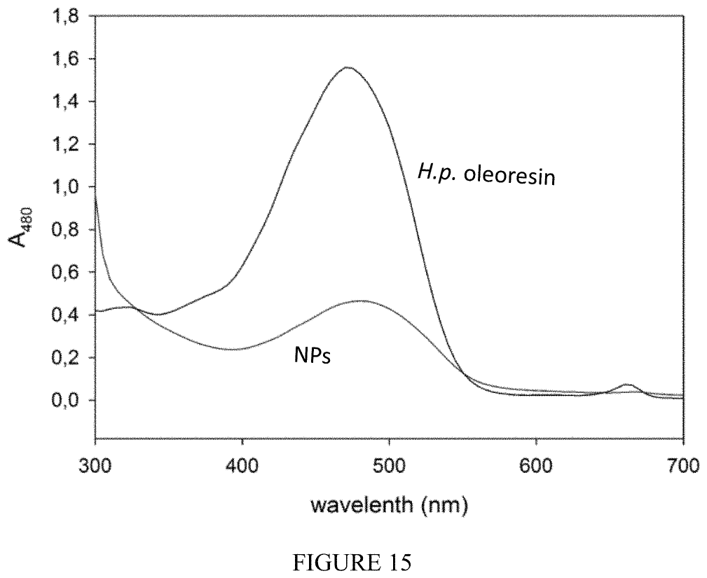

A comparison between the absorption spectra of H.p. oleoresin in ethyl acetate and NPs in water revealed a redshift of the maximum absorption of ASX from 470 nm to 480 nm after encapsulation process (FIG. 15). This might be due to the presence of the protein shell together with the fact that the solvent was necessarily different. In the case of NPs, the great absorption at wavelengths<300 nm is given by the presence of proteins. On the whole the absorption characteristics of encapsulated ASX in the visible spectrum are conserved.

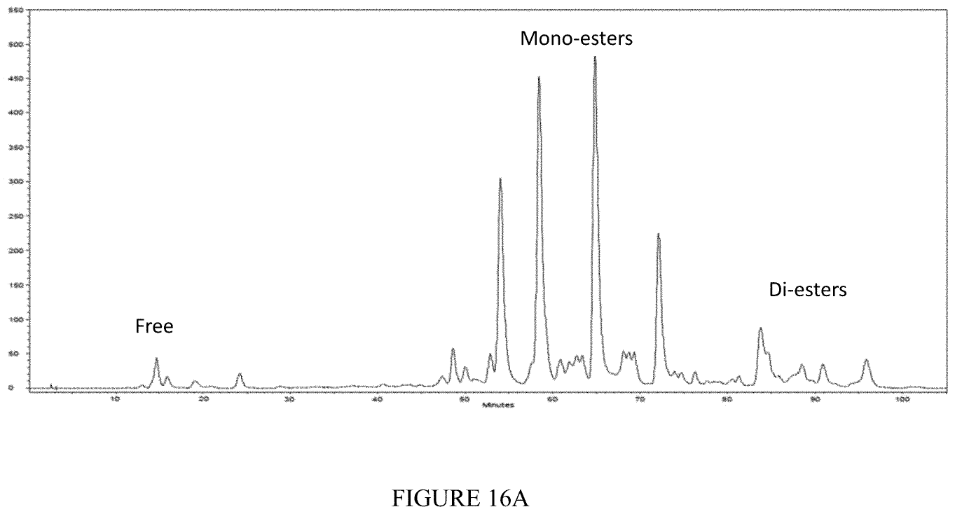

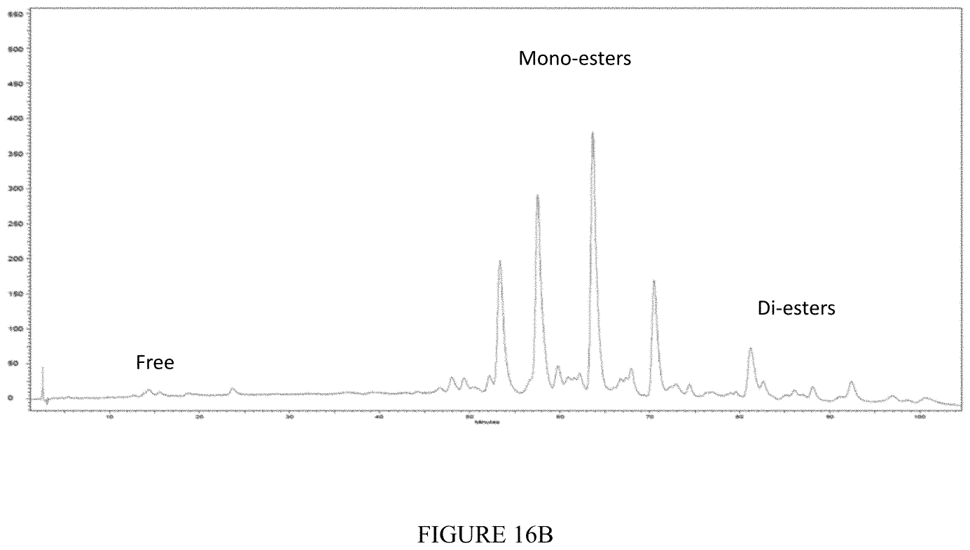

The encapsulation efficiency was 96.0±2.5% with surface ASX accounting only for 0.16±0.02%. The minor loss of ASX could be caused by the oxidation generated by the sonication process, or by the incomplete degradation of the protein shell during the enzymatic digestion that could limit the total solubilization of the carotenoid in the solvent. The HPLC analysis of the extract from optimized NPs was compared to the one of the H.p. oleoresin before the encapsulation process (FIGS. 16A-B). No particular qualitative differences were observed, indicating that encapsulation process did not affect the nature of the esters distribution.

Some works suggested that the radical scavenging activity (RSA) of ASX is mediated by the transfer of hydrogens or electrons, and in the case of the quenching of singlet oxygen, by energy transfer between the electrophilic singlet oxygen and the polyene chain. ABTS represents one of the most used ways to evaluate the RSA of hydrophilic and highly lipophilic molecules such as carotenoids. A concentration of 0.2 mg/ml of ASX from H.p. oleoresin was shown to have a RSA of 72.1%, while NPs, despite presenting 8 times lower ASX concentration (i.e. 0.025 mg/ml Vs 0.2 mg/ml) exhibited a RSA of 95.8% (Table 5). The activity of WPC native proteins was tested and found to contribute up to 74.2% of the total NPs activity. This might be explained taking into account of the scavenging properties of some amino acid residues like cysteine, tyrosine, tryptophan, phenylalanine and histidine present in the proteins structure.

TABLE 5
ABTS radical scavenging activity of H.p. oleoresin and NPs.

| | ASX concentration | |
|---|---|---|
| Sample | (mg/ml) | RSA (%) |
| H.p. oleoresin | 0.2 | 72.1 |
| NPs | 0.025 | 95.8 |

### Example 13

### Stability of the NPs

### Effect of pH

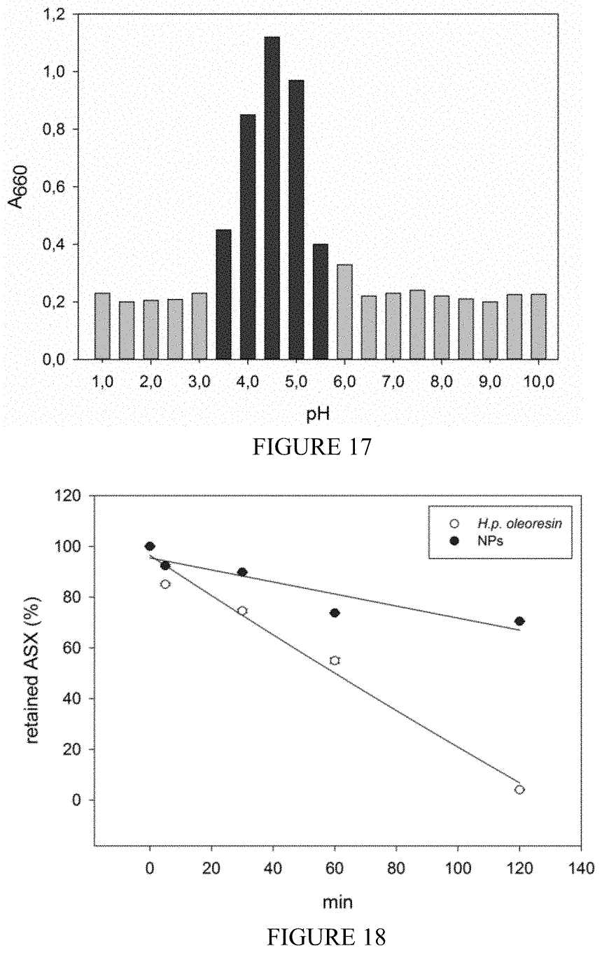

NPs stability was analyzed at different pH values. NPs were found unstable at pH between 3.5 and 5.5, giving the formation of agglomerates that tend to precipitate (FIG. 17). The pH range corresponds to the average isoelectric point of the whey proteins. Qian and co-workers reported that the agglomeration of protein-stabilized nanoemulsion might originate from the small net surface charge registered at pH value close to the pI of the proteins, and thus not sufficient to exert electrostatic repulsion among the particles.

### UV Irradiation

As already reported, one of the major factors responsible for the degradation of ASX is light. Given the importance of this aspect the stability of NPs to UV irradiation was analyzed and compared to H.p. oleoresin. As shown in FIG. 18, after two hours of exposure to UV-B light the percentage of ASX in NPs and in H.p. oleoresin was 70.5% and 4.1% respectively, showing that the protein shell exerts a protective effect. In both cases a zero-order degradation kinetics was observable.

### Fe (III)— Induced Oxidation

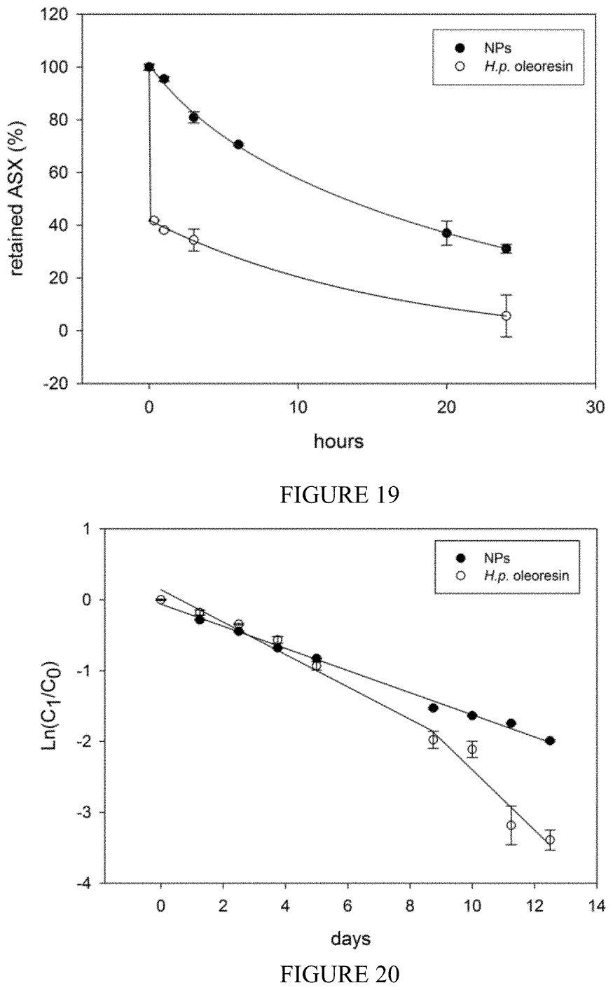

Another factor affecting ASX stability is the presence of pro-oxidant species. Many iron compounds that are ubiquitous in food products are known as harsh oxidizers. The physical barrier represented by the WPC shell and its intrinsic capacity to chelate metal ions could influence the stability of ASX contained in NPs. To this purpose ferric chloride (FeCl₃) is commonly used as an oxidizing agent to study carotenoids stability. The results reported in FIG. 19 show a different behavior between the samples: the H.p. oleoresin displayed a pattern characterized by a rapid degradation kinetics within the first 20 min of exposure with a loss of nearly 40% of the ASX content, followed by a slower rate of degradation until the end of the experiment with the retainment of only 5.6% ASX after 24 h. The NPs showed a slower decrease of ASX compared to the former. Indeed, after 20 minutes the amount of ASX retained was 95%. After 24 hours the amount of ASX was 31%. The results showed a protective effect of the WPC protein shell towards Fe³⁺-mediated degradation.

### Thermal Treatment

Accelerated tests are regularly applied to study the stability of lipophilic substances such as edible oils. This test was employed to study the thermal stability of NPs and H.p. oleoresin (FIG. 20). ASX present in NPs exhibited a typical first-order degradation kinetics, as already observed for many carotenoids. On the contrary, within H.p. oleoresin ASX showed a profile characterized by 2 first-order kinetics: the first one with a lower reaction rate constant, close to that of the oleoresin, and the second one, starting from day 8, with a higher degradation rate. The quicker degradation of H.p. oleoresin could derive from the absence of the protective glassy matrix that allows for a faster accumulation of reactive degradation species originating from the oxidation. When these degradation species reach a certain concentration they could further oxidize the carotenoids present in H.p. oleoresin. A gradual loss of color was observed for both the samples. As reported previously, the auto-oxidation products of carotenoids do not present color properties due to the lack of chromophores at the absorption wavelength of visible light. HPLC analyses of ASX extracted from the NPs and the H.p. oleoresin showed the lack of a selective degradation of ASX: indeed, losses were observed for all the compounds present in the encapsulated H.p. oleoresin. The absence of new peaks detected at 480 nm suggests the conversion of carotenoids into different products. It is reported that the thermal degradation of carotenoids in the presence of oxygen results in the formation of volatile compounds and larger non-volatile compounds. The scarce protection of WPC shell against the thermal treatment could be due to the high surface exposed of the NPs, that could lead to a major exposure of ASX to heat and as a consequence to the degradation of the ASX carbons chain.

### Example 14

### Evaluation of Bioaccessibility by In Vitro Simulated Digestion

The absorption of carotenoids during digestion occurs mainly in the first part of intestine. Lipophilicity of carotenoids is a well know limitation to their uptake, but in the case of ASX, the esterification with fatty acids represents another factor negatively affecting intestinal absorption, since esterified carotenoids are up-taken mainly as free form.

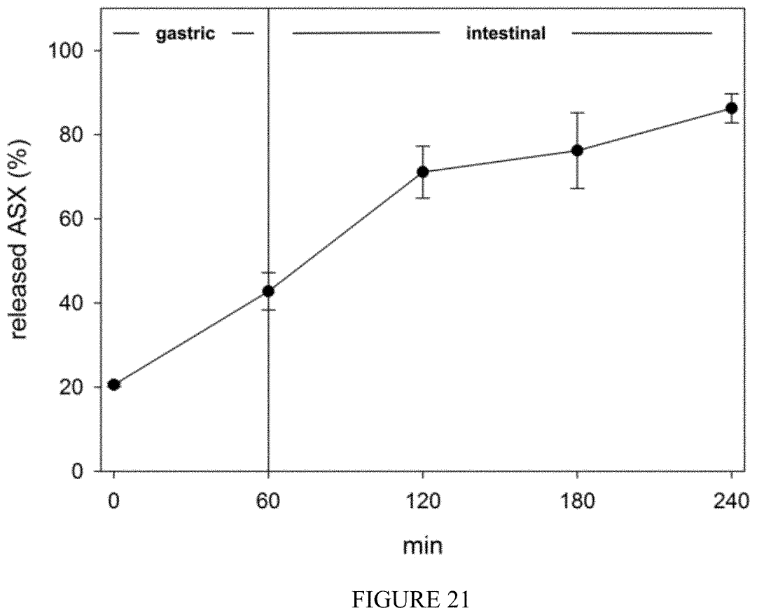

By simulated digestion (SD) experiments it was possible to calculate that the amount of ASX released from NPs, and thus bioaccessible, was about 43% after the gastric phase (FIG. 21). This release was probably induced by the combination of two factors: 1) the activity of pepsin that partially degraded the whey protein shell, and 2) the low pH that can induce the agglomeration of the particles and the destabilization of the protein shell, as proved by the release of 20% of the total ASX at time zero. Attempts to measure the particle size by DLS could not lead to reliable results due to high PDI values.

During the intestinal phase the agglomeration phenomena disappeared as a consequence of the neutralization of pH. This was confirmed also by the average size of the particles, 165.5 nm, and low PDI, 0.290. The release of ASX reached about 76% after 2 hours of intestinal digestion that in this model represents the small intestine. After 3 hours of intestinal digestion the amount of bioaccessible ASX was 86%.

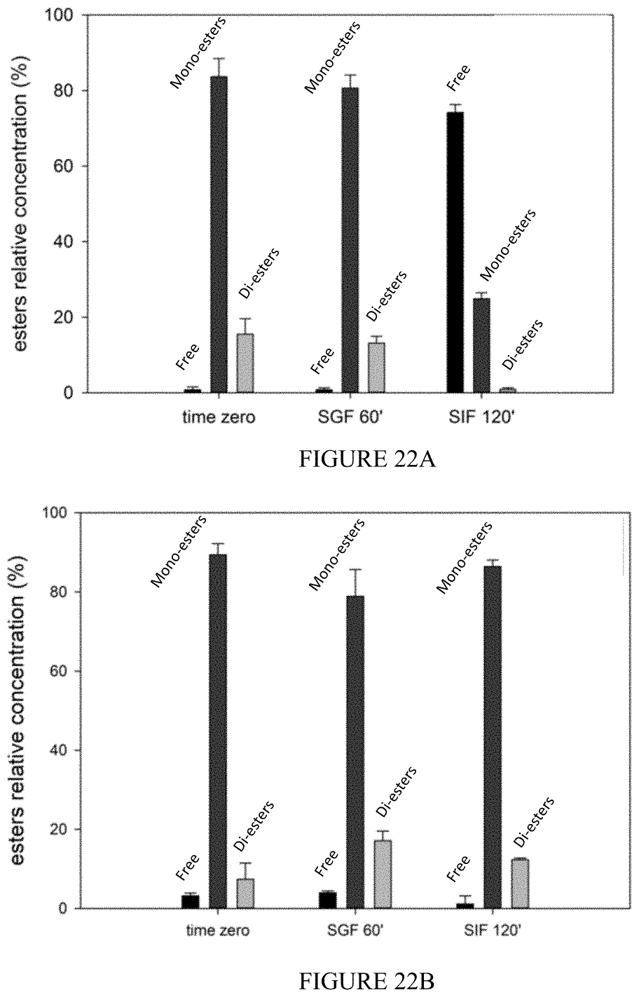

The simulated digestion (SD) was performed not only to address the bioaccessibility of the encapsulated ASX but also to evaluate possible chemical modifications of the carotenoid. HPLC analysis of the oleoresin extracted from NPs collected before and after digestion could give a picture of the variation of the esterification degree of ASX extract. FIGS. 22A-B show that the esters composition before digestion was mainly represented by monoesters and diesters, accounting on the whole for the 99% of the ASX present, while after two hours of the intestinal digestion the major form was represented by free ASX (75%). On the contrary, a sample of H.p. oleoresin diluted in soybean oil treated in the same way gave completely different results, i.e. the relative distribution of the esters was unaffected.

The combination of carotenoids with dietary fats and oils is reported to improve their bioaccessibility by facilitating the transfer to the micelle phase and the micellarization process itself mediated by bile salts. Without being bound to any particular theory, the difference observed between the two samples can be explained considering the dimension of the NPs. Indeed, the higher surface to volume ratio of the NPs could have promoted the greater hydrolysis of astaxanthin esters by lipase, the major enzyme involved in the hydrolysis of triacylglycerols. Another explanation to the loss of hydrolysis in the sample containing H.p. oleoresin can be connected to the great amount of triacylglycerols present that, being the preferential substrate of lipase, might have hampered the activity of this enzyme towards other molecules such as ASX esters.

This result is of great importance because, as mentioned above, the de-esterification of carotenoids is a crucial step required for their uptake through the intestinal mucosa.

### Example 15

### Encapsulation by Fluid Bed Granulation

Several compounds were encapsulated following the granulation process previously described using a fluid bed granulator (Mini-Glatt fluid bed system, Germany). In the process, the evaluation of the solubility of the compound in ethyl acetate is important, in order to avoid the precipitation of the active ingredient.

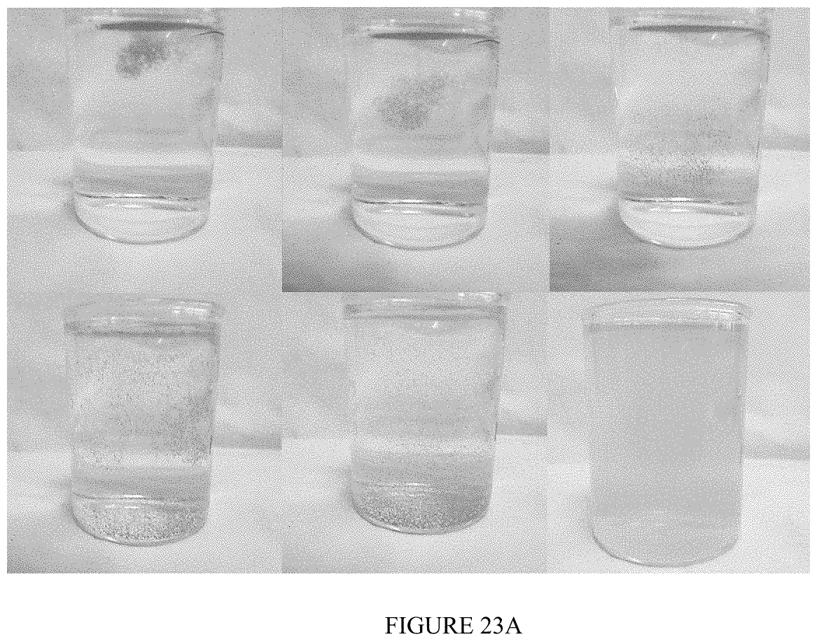

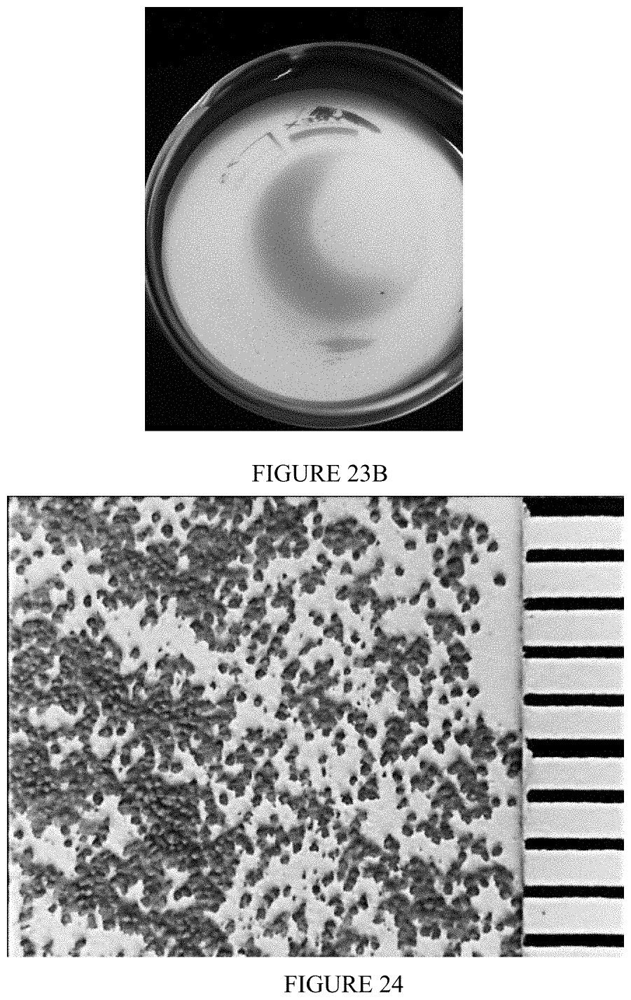

Curcumin (20% w/w) was encapsulated with fava bean proteins. (FIGS. 23A-B and FIG. 24). It can be observed that the dissolution created a homogenous solution with the absence of big visible particles (FIGS. 23A-B).

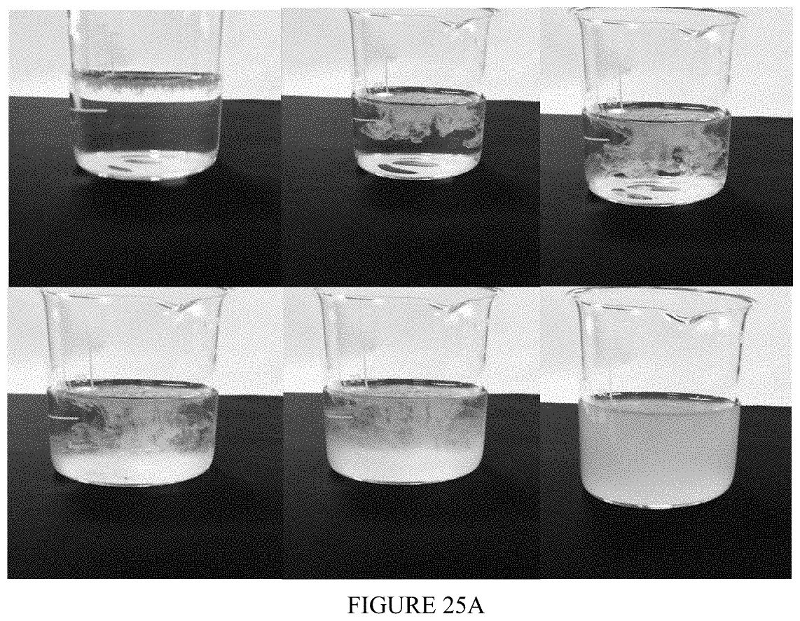

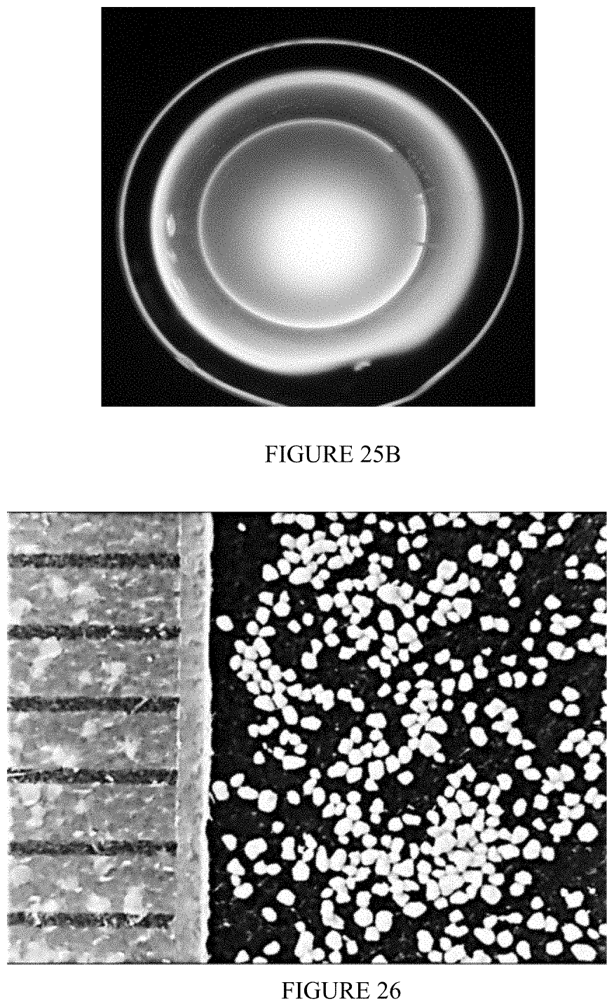

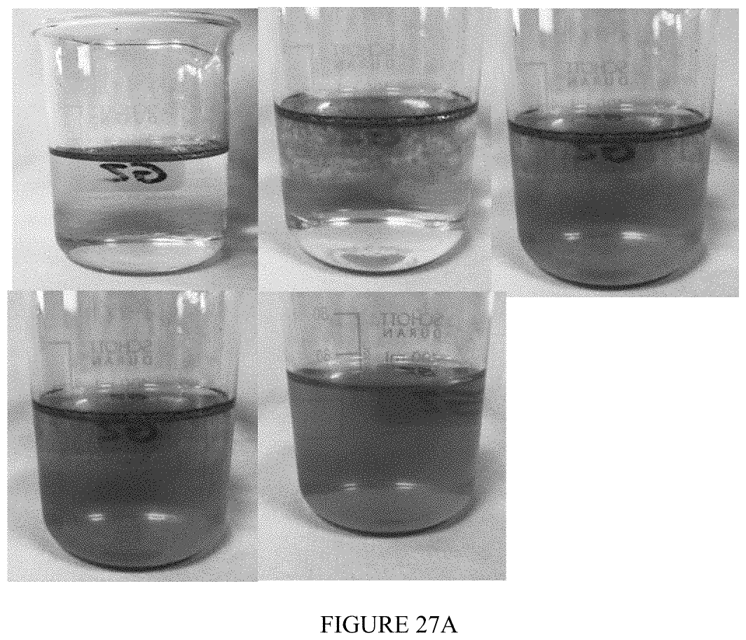

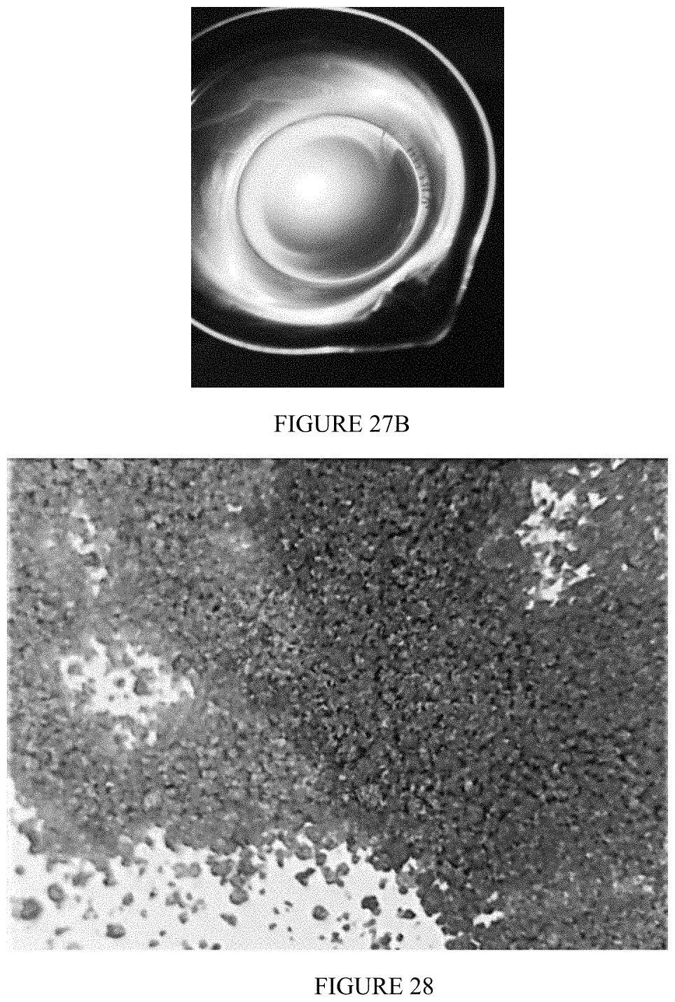

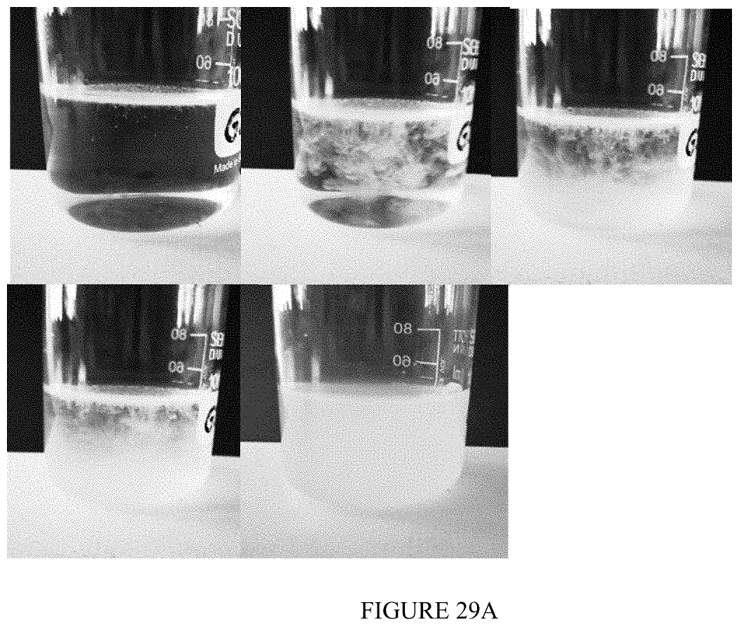

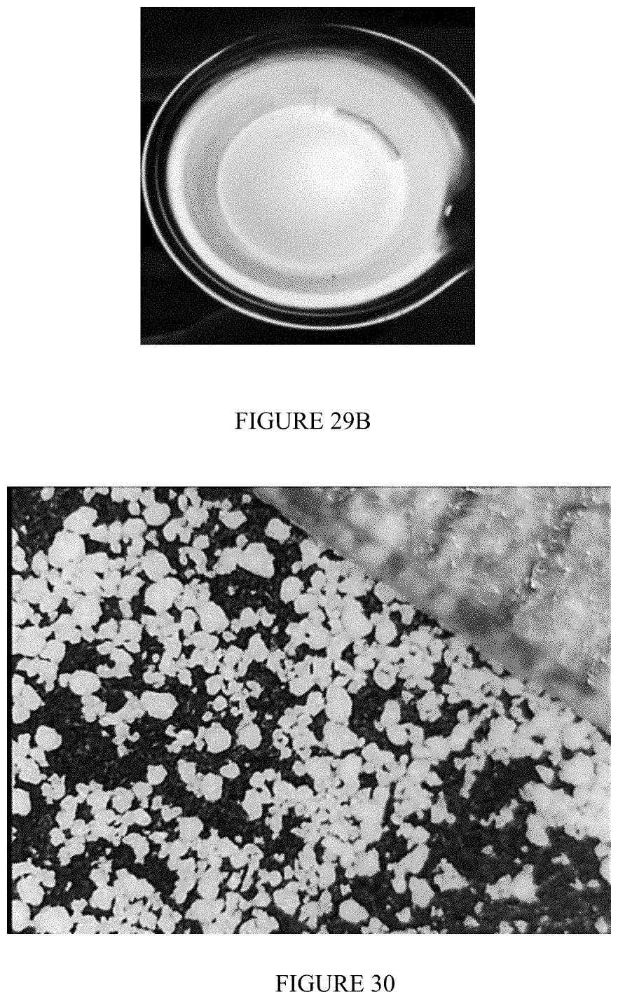

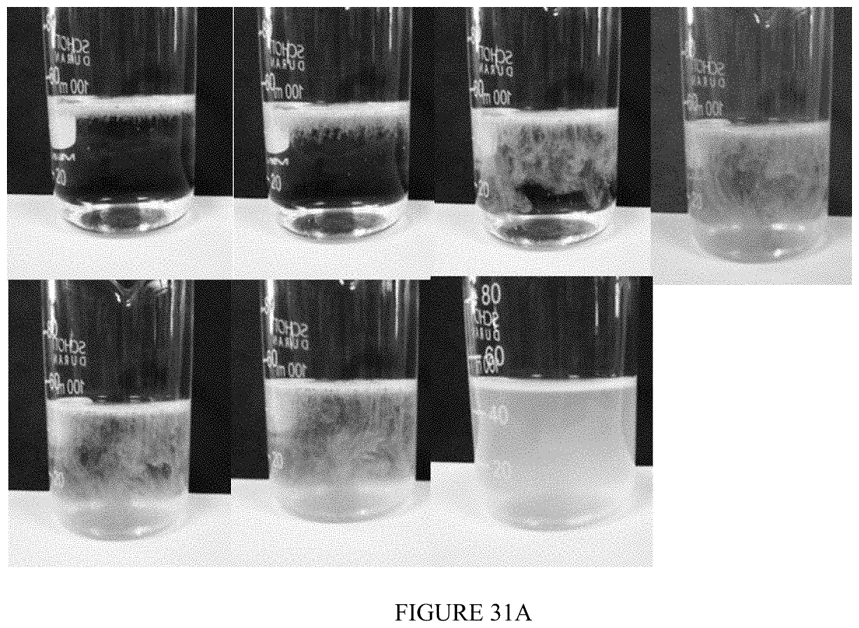

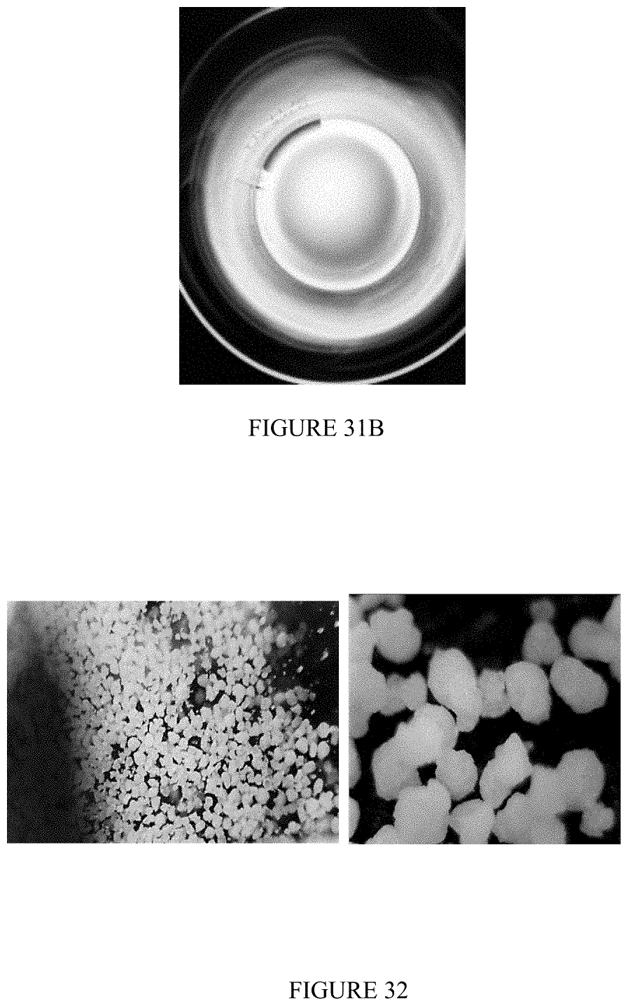

The following compounds 1) Coenzyme Q10 (12.4%±0.1 w/w) (FIGS. 25A-B and FIG. 26); 2) beta-carotene (1.6%±0.03 w/w) (FIGS. 27A-B and FIG. 28); 3) fish oil (10.53%±0.78 w/w) (FIGS. 29A-B and FIG. 30); 4) phytosterol (11.82%±0.07 w/w) (FIGS. 31A-B and FIG. 32); and 5) CBD extract (24%±0.5 w/w) were successfully encapsulated.

### Example 16

### Encapsulation by Spray Dry

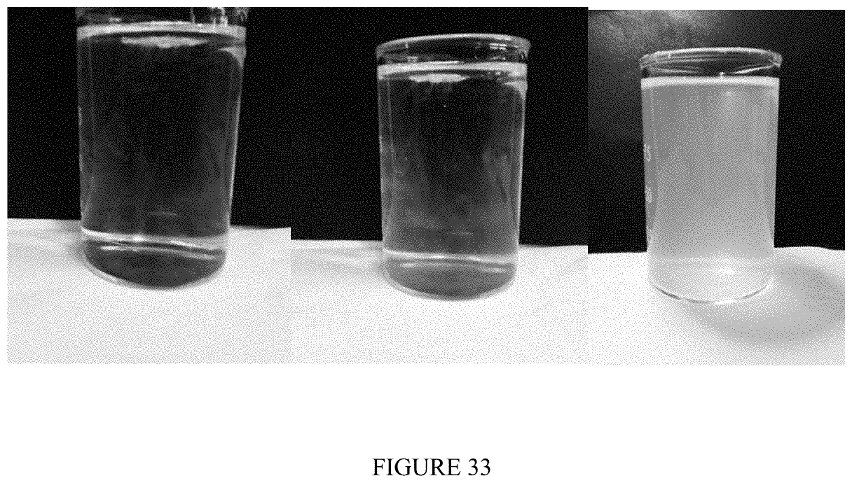

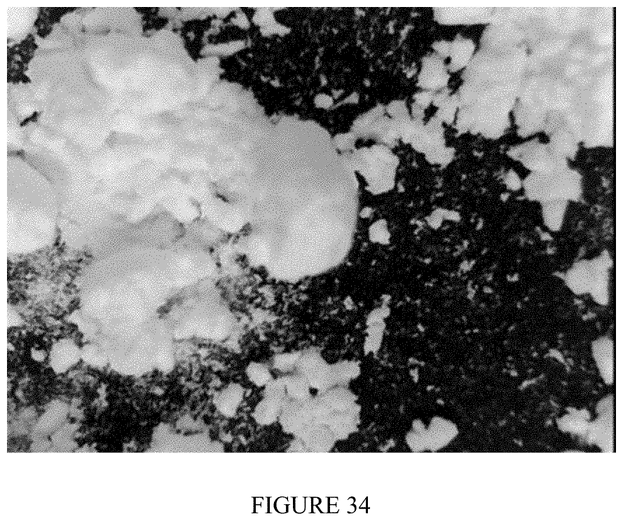

Caffeine (3%±0.17 w/w) (FIGS. 33-34) was successfully encapsulated by spray dry. In order to obtain the caffeine emulsion to feed the spray dry, caffeine was dissolved in ethyl acetate. During the process (NPs formation and production), the evaluation of the solubility of the compound in ethyl acetate is important, in order to avoid the precipitation of the active ingredient. Since caffeine showed low solubility in this solvent, the maximum load reached was only 20 mg/ml. The encapsulant matrices was rehydrated in deionized water for at least 1-hour prior the use, to allow a complete dissolution of the polymer. The polymer was dissolved in distilled water. The caffeine solution was combined with the polymer solution at a ratio of 9:1 (polymer solution: extract). A fine emulsion was produced using an ultra-sonicator for 10 minutes. At the end of the process, ethyl acetate was removed using a rotavapor system. The drying process was performed using a Buchi Mini-Spray dryer B-290 (Switzerland). The condition used were as follows: drying air flow rate 40 m³/h; inlet air temperature 180±3; outlet air temperature 100±3 and a feed flow rate of 4 mL/min. The formed microparticles were collected in the collector at the bottom of the cyclone separator.

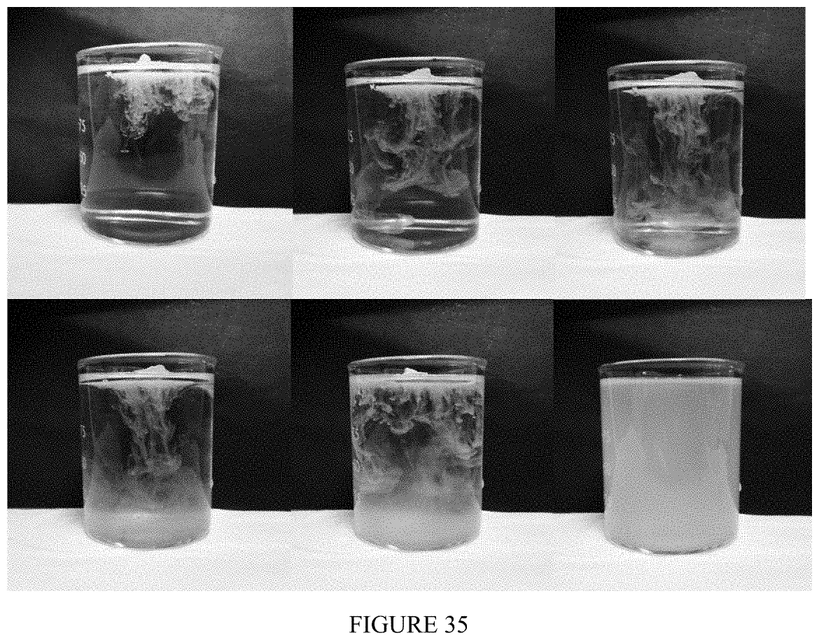

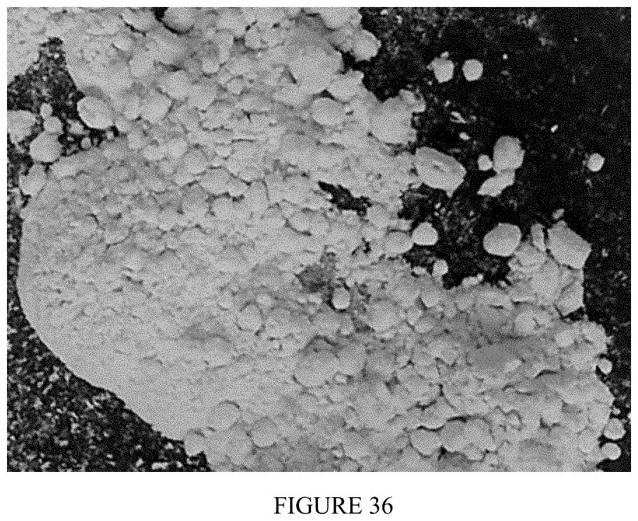

The same process was applied for epigallocatechin gallate. Since the solubility in ethyl acetate of this compound is higher, the final loading obtained in the powder was also higher. epigallocatechin gallate (24%±0.03 w/w) (FIGS. 35-36) was successfully encapsulated by spray dry.

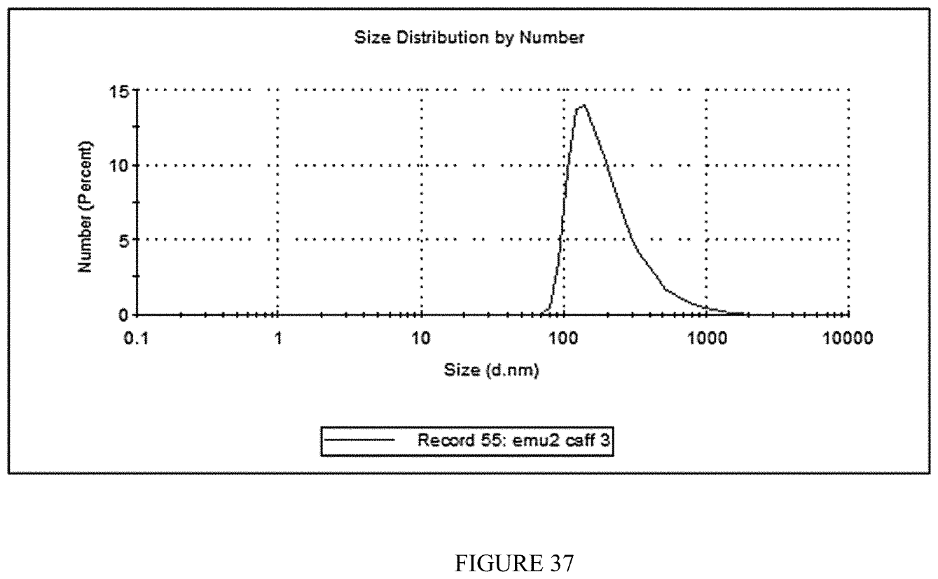

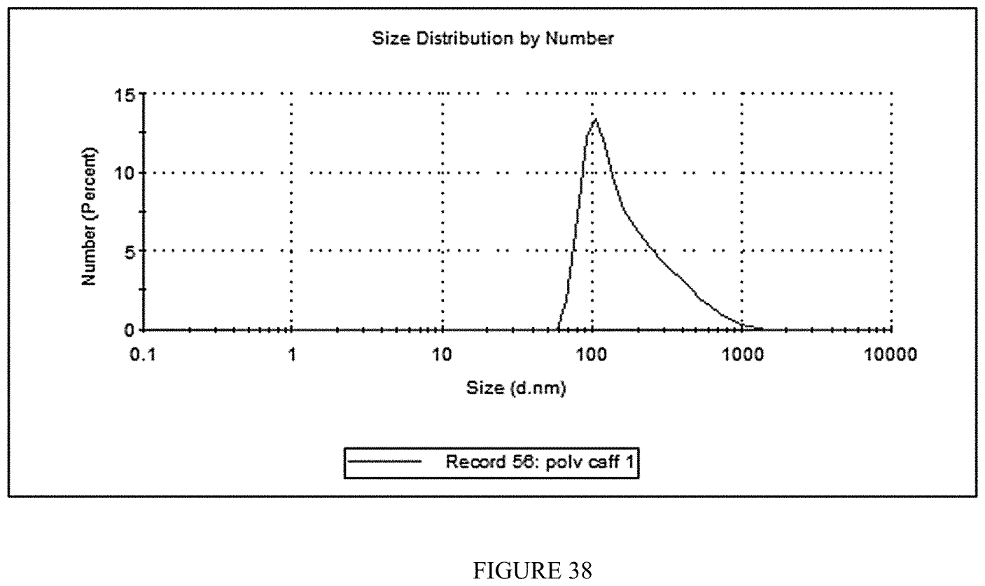

A DLS analysis was done for an emulsion obtained with WPC and 3% of caffeine, and corresponding powder form (FIGS. 37-38), both showing a low PDI of 0.348 and 0.244 respectively.

It can be observed that the formulation keeps the same dimension before and after the drying process. This is an indication that the obtained formulation is very stable and can sustain the high pressure and harsh conditions of the drying process.

### Example 17

### Antioxidant Capacity of Astaxanthin Nanoparticles

ASX NPs were obtained through emulsion solvent-evaporation technique with whey protein concentrate as encapsulant matrix. The obtained particles, were stable in solution, characterized by a highly negative Z-potential value (−28.5 mV), an average diameter of 90-100 nm) and a low polydispersity index (PDI) (0.245), underlining the presence of a slightly polydisperse population of NPs.

### Antioxidant Capacity

A concentration of 0.2 mg/ml of ASX from H.p. oleoresin was shown to have a Trolox Equivalent Antioxidant Capacity (TEAC) value of 30 (expressed as mmol Trolox/kg extract), between 6 and 9 times lower than the values found in literature, probably because of the low extraction efficiency of ASX or for the differences in the algae batch used. ASX NPs displayed an 11-folds higher antioxidant capacity than H.p. oleoresin tested at the same concentration. This result could be explained considering the antioxidant properties of whey proteins, and the small diameter of the NPs, if compared to the crystalline form of non-encapsulated H.p. oleoresin, that might increase the surface to volume ratio. The antioxidant capacity (AOC) of WPC native proteins were tested and found to contribute for the 74.2% of the total ASX NPs antioxidant capacity.

### Cellular Antioxidant Activity of WPC ASX NPs

The AOC of WPC ASX NPs was tested also through the use of model cell line.

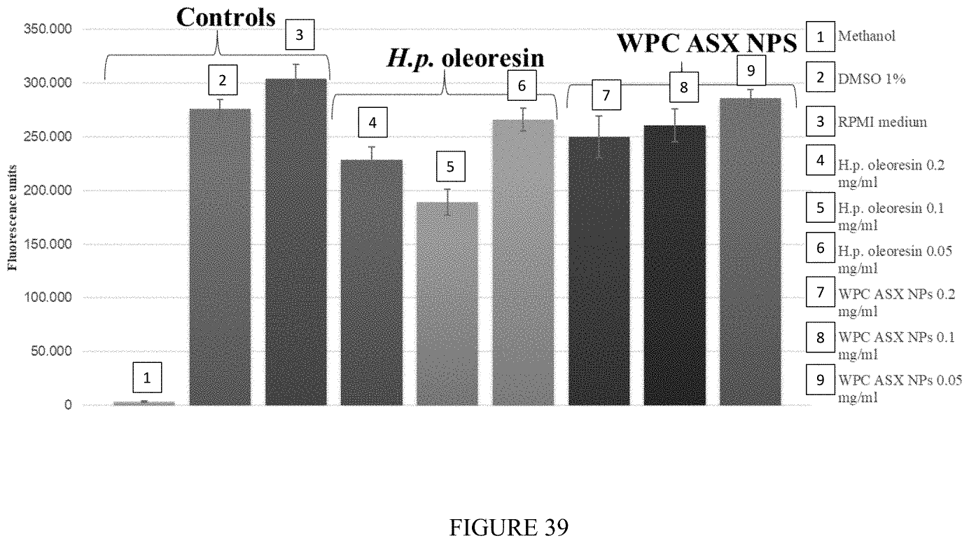

Initially, the cytotoxicity of H.p. oleoresin and WPC NPs were tested at three different concentrations (0.2, 0.1 and 0.05 mg/ml) on HepG2 cells. The obtained results (FIG. 39) showed negligible toxicity effect of WPC ASX NPs and a slight viability decrease of the cells treated with H.p. oleoresin at 0.1 mg/ml.

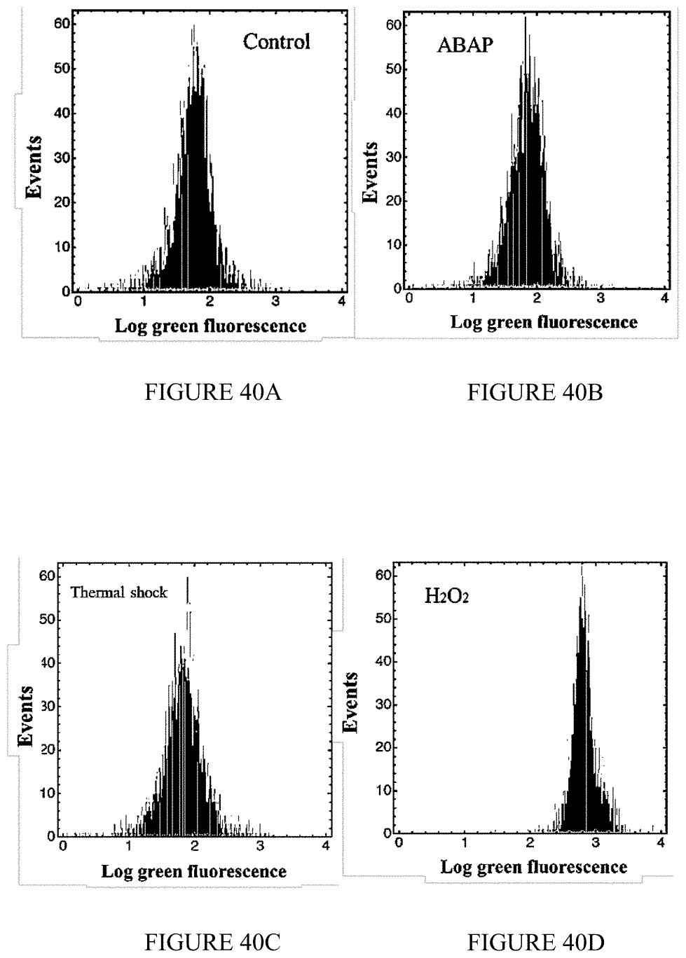

Cellular antioxidant activity (CAA) was developed by Wolfe and co-workers. It is one of the most employed antioxidant techniques used to study the effect of antioxidant substances in cells systems in presence of highly reactive species. Although this technique is well established into microplate experimental set up using HepG2 cells, our attempts to apply this system to our samples did not lead to consistent and reproducible results. For this reason, and because the method previously mentioned does not provide any information about cells viability during the experiments, a different approach based on flow cytometry was chosen. This approach gives the opportunity to discriminate between viable and non-viable cells due to their light forward and side scattering properties. To perform the assay, a macrophages cell line from adult mice (J774A.1) was selected. Compared to HepG2, frequently employed for the CAA assay, J774A.1 cells present higher phagocytic activity and are easier to manipulate. Moreover, they are naturally capable to produce high amounts of ROS. The first step to develop the method was to identify an appropriate stimulus for the generation of ROS by the cells. Macrophages cells were subjected to different treatments: thermal shock, and incubation with different chemicals, e.g. ABAP, H₂O₂. The generation of ROS would then oxidize DCFH-DA with the concomitant emission of green fluorescence. Among the treatments, as shown in FIGS. 40A-D, the fluorescence distribution relative to the treatment with ABAP (FIG. 40B) and thermal shock (FIG. 40C) were close to the signal produced by the control sample (FIG. 40A). In the case of cells treated with H₂O₂ (FIG. 40D) the fluorescence signal was largely increased in respect to the control, underlining a higher production of ROS species by these stressed cells. For this reason the treatment with H₂O₂ was chosen to induce a strong and homogeneous production of ROS species, in terms of time.

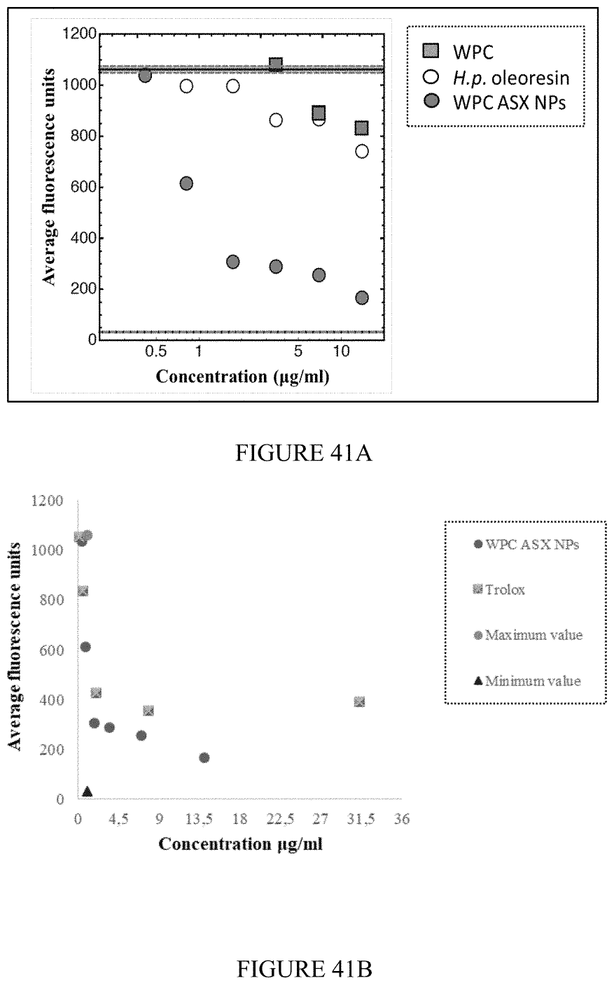

As shown from the fluorescence data, in FIG. 41A WPC ASX NPs were able to inhibit the fluorescence emission in a dose-dependent manner more effectively than WPC alone in the form of native proteins and H.p. oleoresin. In particular, WPC ASX NPs showed an AOC respectively 4 times higher than H.p. oleoresin at the maximum concentration tested (14 μg/ml) and 5 times higher than 1% WPC solution. It was interesting to observe that, differently from ABTS, by this system WPC did not show antioxidant properties, and all the activity seems to be related to ASX. The higher CAA showed by the nano-encapsulated system might derive from the higher uptake of ASX by the cells in respect of the H.p. oleoresin form. In this contest, another important point to underline is the fact that the dispersion of H.p. oleoresin solubilized in DMSO could have formed aggregate when in contact to aqueous media that had led to the formation of aggregate with larger size in respect to the NPs system reducing in this way the absorption of the active ingredient inside the cells.

The antioxidant capacity of NPs was compared also with Trolox, as shown in FIG. 41B. ASX in the form of NPs showed higher antioxidant activity at all the tested concentrations.

### Cellular Uptake of ASX NPs

Testing the bioavailability of NPs by in vivo systems is difficult to perform from a practical and ethical point of view. In this way, in the last decade, in vitro models, such as cellular uptake analysis through confocal microscopy systems had gained much interest due to the fact that it can provide useful information about the potential fate of NPs in a complex system. It is also relatively easy, quick to perform, and it allows for relatively inexpensive screenings of multiple samples.

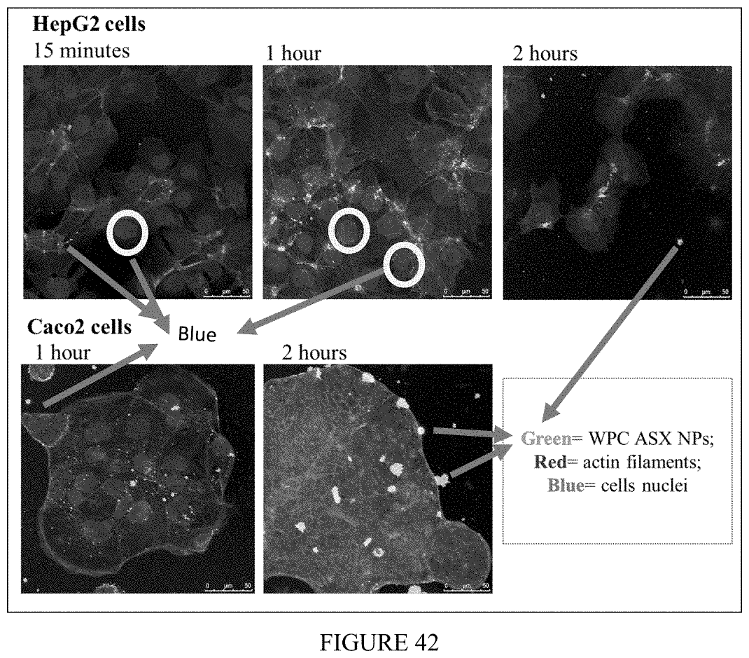

FIG. 42 shows the micrograph pictures obtained by confocal microscope. In particular, concerning HepG2 cells, ASX NPs are visible inside the cells after 15 minutes of incubation underling that the uptake process of NPs is very fast, the accumulation seems to proceed later on during the first and the second hour of incubation as can be observed by the slight increase of fluorescence.

ASX NPs uptake is visible also in Caco2 cells. NPs appear to accumulate close to the cell membrane leading to bigger aggregates after 2 hours.

### Inhibition Study

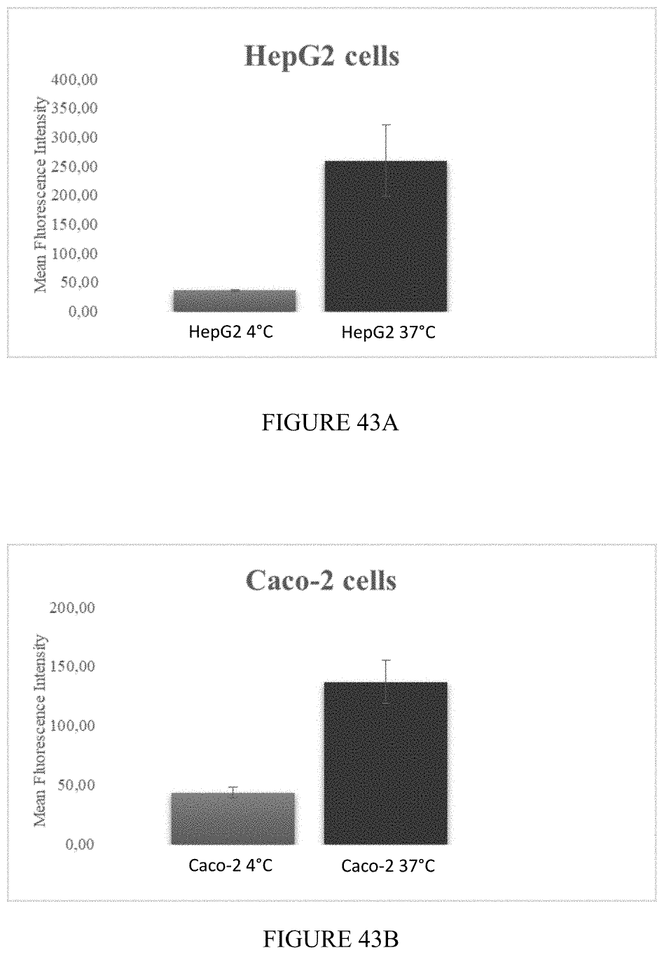

To study the cellular uptake mechanism in the case of ASX NPs, the uptake was tested in presence of an endocytic blocking condition for both HepG2 and Caco2 cells. At 4° C., the conditions tested, all the energy-dependent processes, and thus also endocytosis, are inhibited. In FIGS. 43A-B, HepG2 cells showed a NPs uptake inhibition percentage of 86%, Caco2 cells showed an inhibition of 68%. The lower uptake could be due to the reduced cell activity and to the scarce fluidity of the cell membrane. The uptake still measured could be explained by the presence of residual ATP, that can be used for the transportation of the NPs in the cells. In particular, during endocytic internalization it was reported the polymerization and rearrangement of actin filaments when the process was caveolae- and clathrin-mediated, this seems to validate our previous observations about the peculiar positioning of NPs along actin filament during cellular uptake in the case of HepG2 cells (FIG. 42).

### Example 18

### Evaluation of Different Plants Proteins for the Nano-Encapsulation of H.p. Oleoresin

### Chemicals and Reagents

Soya protein isolate (SPI) was purchased from ACEF (Milano, Italy). The protein composition (w:w) was protein 80%. Pea protein isolate (PPI) and rice protein isolate (RPI) were purchased from Raab Vitalfood (Rohrbach, Germany). PPI protein composition was 80% (w/w). RPI protein composition was 80% (w/w). Haematococcus pluvialis powder was purchased from a local supplier (Italy). Ethyl acetate, acetone HPLC-grade, pepsin, pancreatin, trypsin and sodium cholate were purchased from Sigma-Aldrich (St. Louis, Mo., USA).

### Superficial Astaxanthin

A volume of 0.5 ml of NPs was extracted with 1 ml of ethyl acetate by continuous agitation for 10 minutes. The sample was centrifuged at 12.000 rpm for 5 minutes. The supernatant was collected, diluted and analyzed by a spectrophotometer as previously described.

SPI, PPI and RPI were employed as alternative encapsulant matrices to improve the water-dispersibility and bioavailability of ASX through emulsion-solvent evaporation technique. Plant proteins are often better accepted by consumers since they can comply with specific cultural and religious indications, i.e. vegetarian, vegan, lactose-free and kosher. In addition, in compliance with the EU regulation (Annex II of the EU Food Information for Consumers Regulation No. 1169/2011 and Commission Delegated Regulation (EU) No. 78/2014 amending Annex II to Regulation (EU) No 1169/2011), pea and rice proteins are not considered as allergenic sources.

### Production of SPI and PPI ASX NPs

Using the same approach previously described, the optimization of the encapsulation parameters for SPI and PPI started from the study of the variation of dimensions and z-potential of the NPs as a function of the concentration of protein and H.p. oleoresin. The first parameter considered, i.e. the concentration of proteins needed to encapsulate ASX, is crucial since the presence of free unabsorbed protein molecules in the continuous phase may promote depletion and flocculation of oil droplets.

### ASX SPI NPs

SPI are currently one of the most abundant plant proteins. They exhibit high nutritional value and desirable functional properties as emulsifiers and texturizing agents. From the chemical point of view they are composed by a balanced composition of polar, non-polar and charged amino acids, and thus they are able to incorporate molecules with different chemical characteristics. The major fraction of SPI is composed by glycinin and β-conglycinin.

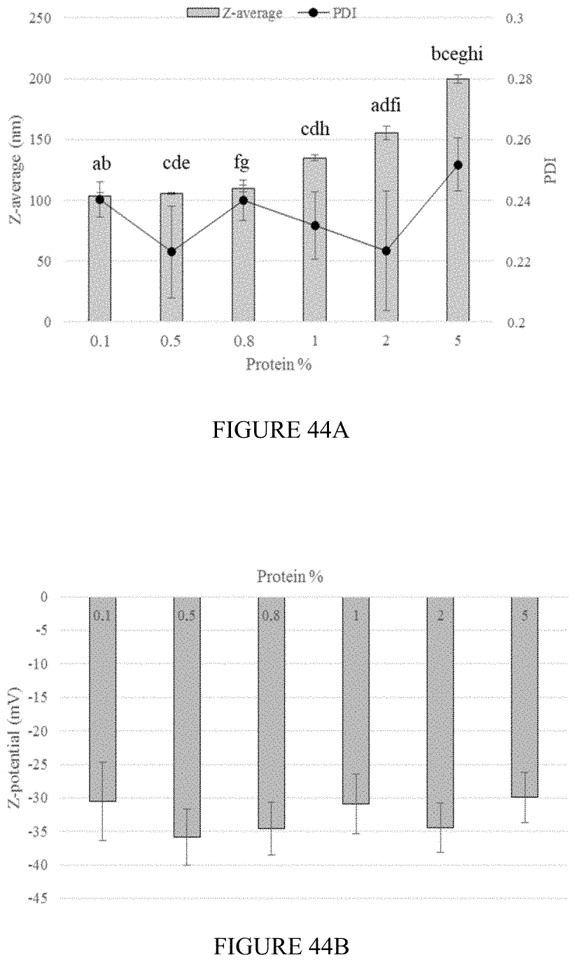

In the case of SPI ASX NPs production, differently from WPC, it was impossible to obtain NPs using solutions more concentrated than 5% due to the limited protein solubility (see FIGS. 44A-B). Average size, PDI and zeta-potential were considered as reference parameters to evaluate the performances of the process. In the first experiment, all the NPs were produced keeping constant the amount of H.p. oleoresin (1%) and varying the amount of encapsulant matrix. As presented in FIG. 44A, the dimensions ranged from 103 to 200 nm, confirming the strong dependence of size from proteins concentration, and the tendency of plant proteins to give bigger NPs probably due to the higher interfacial tension given by the higher tendency of this proteins to bind water, increasing as a consequence also the viscosity of the solution, rigid structure, higher molecular weights that renders the diffusion of proteins slower through the oil/water interface and the lower presence of hydrophobic amino acids in comparison to dairy proteins. PDI values were acceptable for all the formulations ranging from 0.24 (0.1% SPI) to 0.25 (5% SPI), describing moderate polydisperse samples. As already mentioned, zeta potential values higher than +20 and lower than −20 mV are good indicators of NPs stability, SPI ASX NPs displayed highly negative values for all the samples (FIG. 44B), from −30 to −35.8 mV. The more negative values compared to those obtained with WPC, are due to the chemical structure of soy proteins and the higher presence of negatively charged groups at neutral pH.

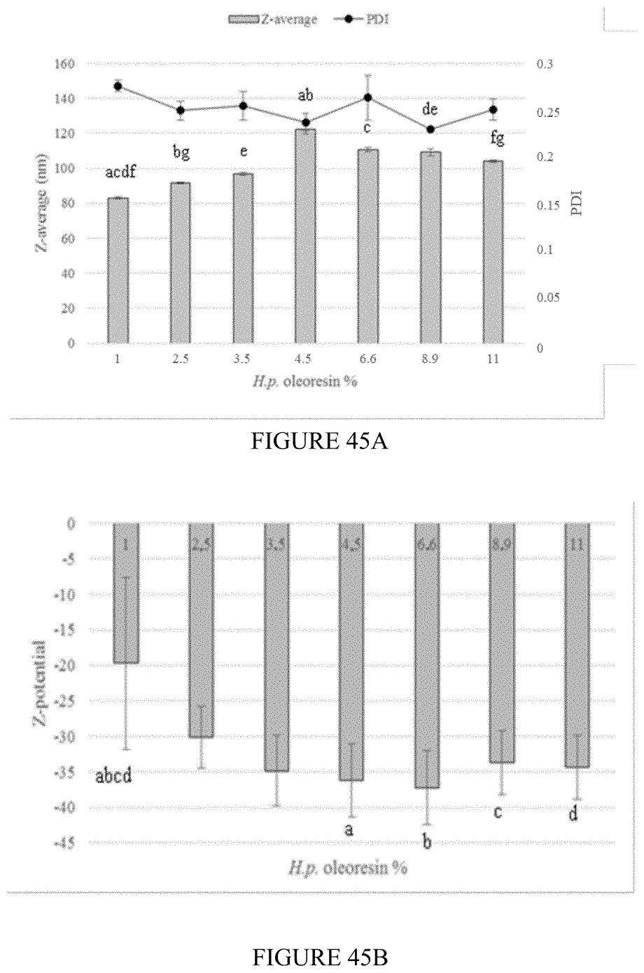

In order to evaluate the effect of H.p. oleoresin concentration on NPs production, SPI concentration was set to 1%. Differently from when using WPC, increasing concentrations of oleoresin led to the growth of NPs dimensions (FIG. 45A), from 83 nm using 1% oleoresin to 125 nm with 4.5% oleoresin. Increasing further the concentration of oleoresin resulted in an unexpected decrease of the diameter. PDI values were in the range of 0.23-0.28. Like in the case of WPC, no particular trends could be appreciated, and no correlation of PDI to the diameter was found. The sample at 1% oleoresin gave the less negative Z-potential value (−19.7 mV) (FIG. 45B). This last result could be explained taking into account that a specific ratio between the protein acting as emulsifier and the lipophilic molecules should be respected. Probably, in the case of the sample composed by 1% proteins and 1% H.p. oleoresin, the amount of proteins was too much higher in comparison to the oleoresin.

The interaction of this free protein fraction together with the neo-produced NPs could result in a partition neutralization of the superficial charge, thus explaining the low Z-potential for this sample. With the increased amount of H.p. oleoresin, the decrease of the ratio protein/oleoresin could allow for the migration of the proteins to the oil droplet surfaces, producing the higher charge measured for these samples.

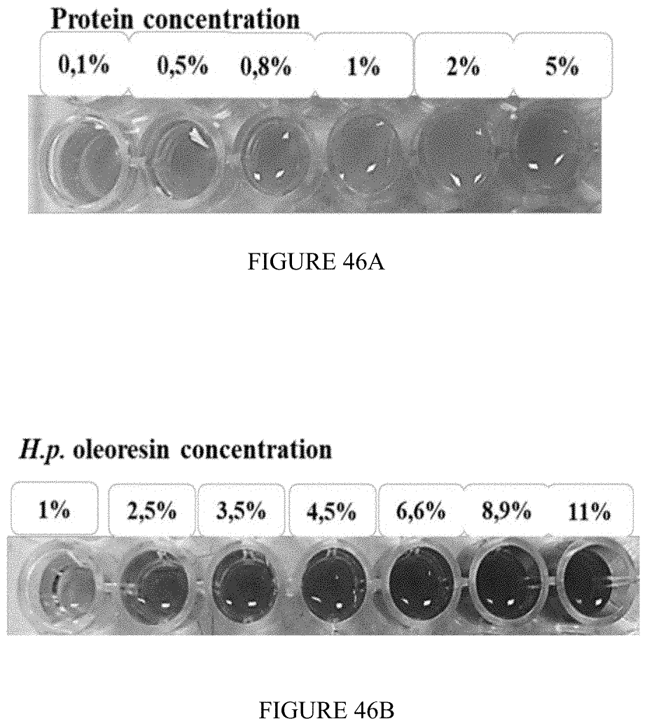

The ASX SPI NPs obtained (FIGS. 46A-B) were characterized by a red-orange color and a slight opalescence, especially at high protein concentration (FIG. 46A). Opalescence in this type of system can arise from the presence of particles characterized by high particle size (higher than 100 nm) or from the interactions among proteins that tend to form aggregates.

### ASX PPI NPs

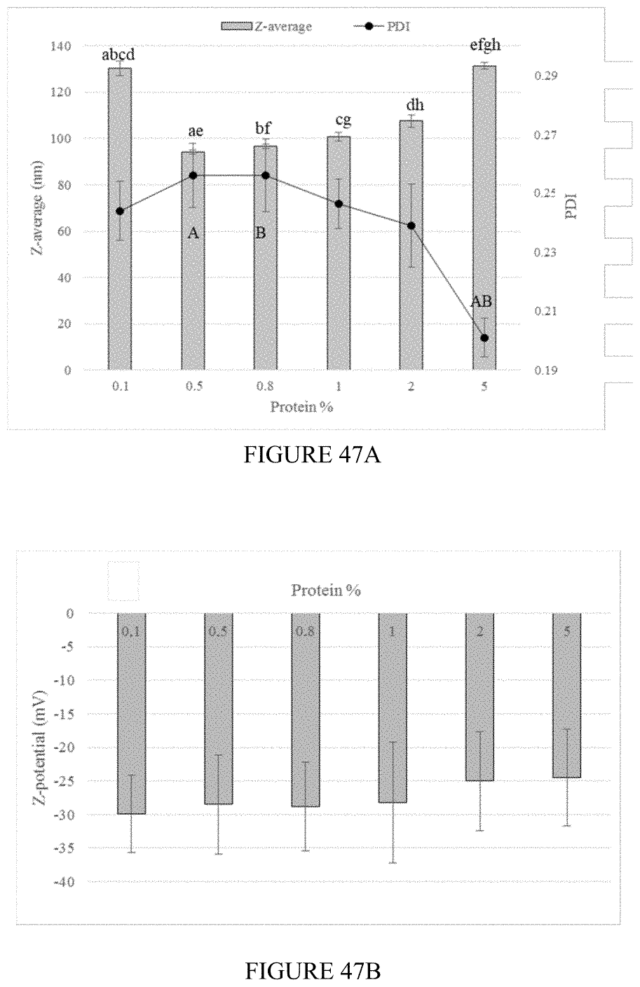

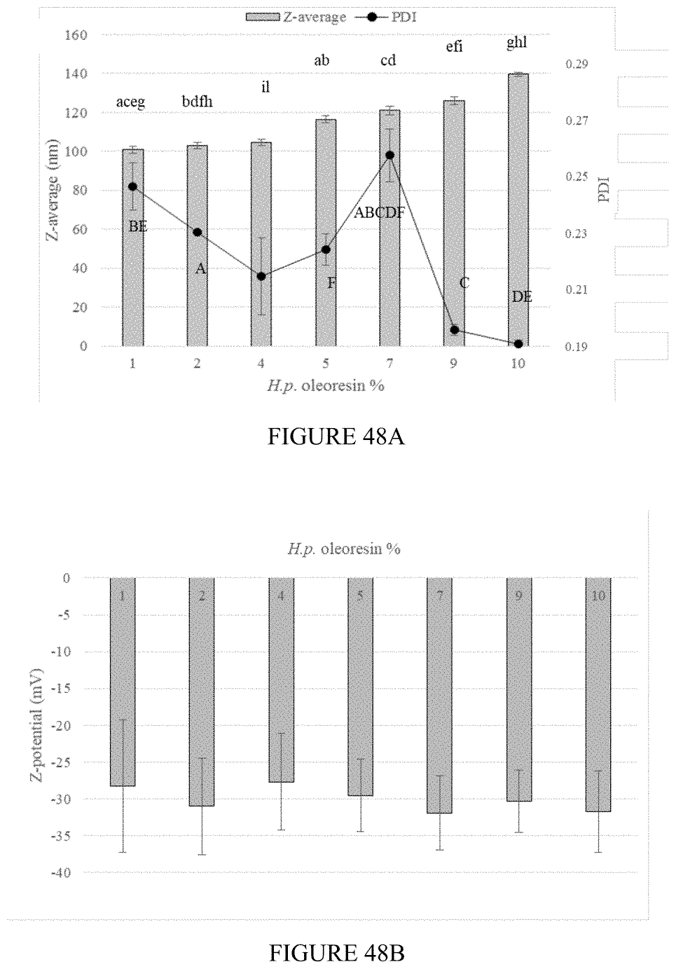

The same analyses were conducted also for the nanoencapsulation of ASX using PPI as encapsulant matrix. The main fraction of PPI is composed by legumin (60,000 Da), vicilin (50,000 Da) and convicilin (70,000). As in the case of SPI, the percentage of protein concentration tested was from 0.1 to 5% due to the fact that higher protein concentrations led to the production of a very viscous solutions not suitable for our purpose. As shown in FIG. 47A, the average diameter of ASX PPI NPs obtained ranged from 94 to 130 nm, with the NPs characterized by bigger dimensions produced respectively with the 0.1 and 5% protein concentrations. Z-potential ranged from −29.9 to −24.4 mV, underlying that all the NPs produced were characterized by a highly negative charge, and thus stability against flocculation and aggregation phenomena (FIG. 47B). No correlation was found between protein concentration and Z-potential. Keeping protein concentration constant (1%), by increasing the amount of H.p. oleoresin the dimensions of the obtained ASX PPI NPs ranged from 100 to 140 nm (FIG. 48A), where the dimension of the NPs produced was correlated to the % of oleoresin, confirming the trend already observed in the case of ASX SPI NPs. The PDI was comprised between 0.21 and 0.25 for the preparation containing H.p. oleoresin from 1 to 5%, meanwhile the NPs produced with 7% of H.p. oleoresin showed a higher PDI, i.e. 0.257, that can be still considered associated to a moderate polydisperse system. For the NPs produced with the highest concentration of H.p. oleoresin (9 and 10%) a significantly lower PDI was unsuspectedly observed (i.e. 0.19). The lower protein to oleoresin ratio of these samples produced a more homogeneous NP population with bigger average size. The surface charge was highly negative for all the samples, from −27.7 to −31.9 mV (FIG. 48B), without an evident dependency on the amount of H.p. oleoresin.

### Evaluation of Plant Proteins Encapsulation Properties

Commercial proteins isolates are commonly used as emulsifier in emulsification-solvent evaporation technique but sometimes they show poor solubility in the aqueous phase compared to the laboratory purified proteins, thus limiting the possibility to further scale-up the process. A typical problem associated to isolates is the possible incomplete dissolution of the matrix that could generate the production of big particles and agglomerates, hampering the efficiency of the process. For this reason, the inventors decided to study the encapsulation properties of proteins subjected to different pre-treatments: heat treatment, adjustment of pH to values far from isoelectric points of the proteins, and the combination of these two parameters.

### Soya Protein Isolate (SPI)

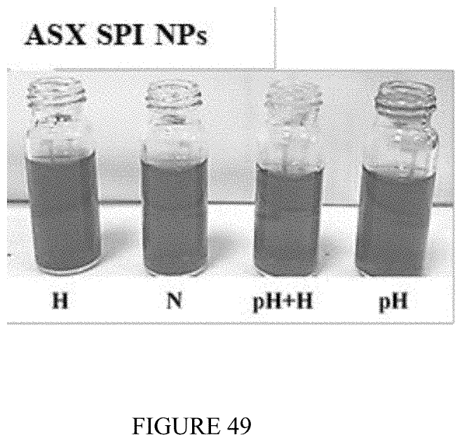

Comparative tests were performed in order to understand if the pre-treatments (heat, pH and a combination of the two) of proteins before encapsulation could have an effect on their solubility, and as a consequence, on the encapsulation efficiency of ASX. FIG. 49 shows the obtained ASX SPI NPs: all the solutions were transparent and show orange-red bright color.

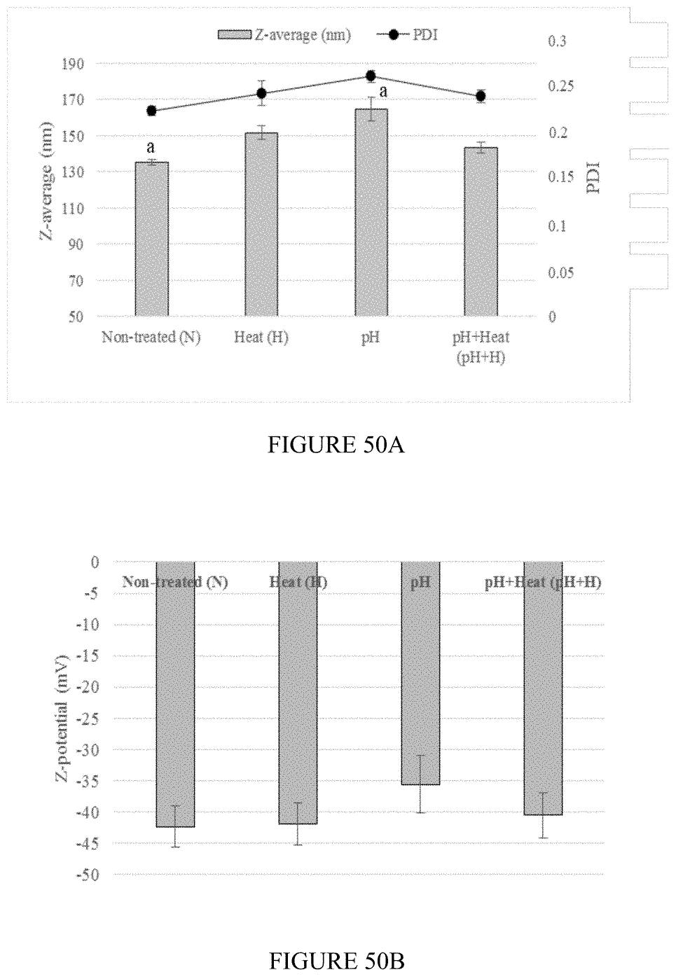

The smallest diameter was obtained with the non-treated proteins (N) i.e., 135 nm, the biggest diameter with the proteins solubilized at pH 8, i.e. 164.6 nm (FIG. 50A). PDI values were in the range of 0.22 and 0.26 for all the preparations. Z-potential values (FIG. 50B) were highly negative for all the preparations, underling a substantial stability of all the ASX SPI NPs prepared.

In Table 6 two significant parameters for the evaluation of the encapsulation process are reported: the encapsulation efficiency (EE), referred to the amount of ASX effectively encapsulated inside the NPs, and the superficial ASX (sASX), that describes the amount of ASX present on the NPs surface. sASX could easily undergo oxidation reactions losing its structural integrity and functionality. For this reason, a very low value of superficial ASX is recommended.

TABLE 6
Effects of the pretreatments on encapsulation efficiency
(EE %) and superficial ASX % of ASX SPI NPs. Differences
between values indicated by the same letter are statistically
significant (P < 0.05).

| ASX SPI NPs | EE % | sASX % |
|---|---|---|
| Non-treated | 93 ± 0.0 l^{abc} | 0.0037 ± 0.0001^{AB} |
| Heat | 99 ± 1.03^{a} | 0.0097 ± 0.0004^{C} |
| pH | 99 ± 0.85^{b} | 0.0261 ± 0.0023^{AC} |
| pH ± heat | 96 ± 0.14^{c} | 0.0153 ± 0.0004^{B} |

The EE was high for all the samples, with the highest value of 99% obtained for the heat-treated sample. The lowest values were obtained for the sample N with an EE of 93%. The higher EE displayed by sample H could be a consequence of the temperature-induced protein conformational changes that could allow to better allocate the oil phase. The amount of superficial ASX was negligible for all the samples: from 0.026 to 0.0037%.

### Pea Protein Isolate (PPI)

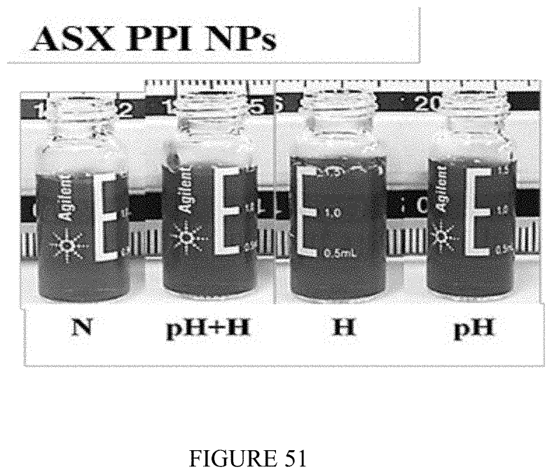

The appearance of ASX PPI NPs is shown in FIG. 51. The solutions were transparent and orange-red in color. No evident differences were observable among the samples.

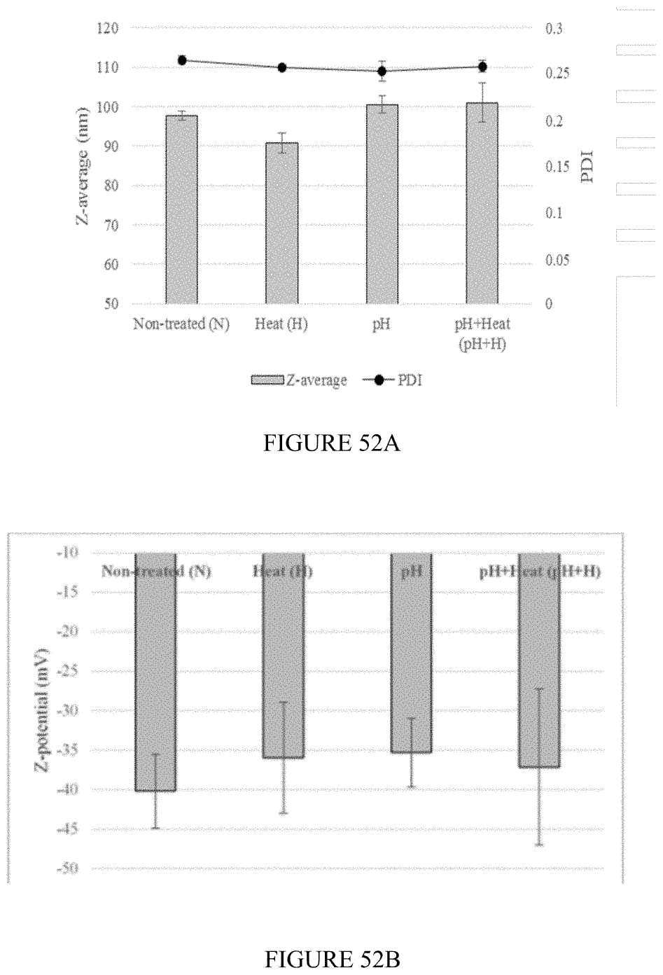

The average sizes of the obtained NPs (FIG. 52A) were smaller than those obtained with SPI and much more similar to those obtained with WPC. Sample H gave the NPs with the smallest average dimension (91 nm), pH and pH+heat treated samples gave the bigger NPs diameters (around 100 nm). PDI values were very similar for all the samples, ranging from 0.25 to 0.265, underling that probably the pre-treatment of proteins did not have a considerable effect on the size distribution of ASX PPI NPs. Z-potential values were highly negative for all the samples (FIG. 52B), and no significant difference was observable between the samples. EE % ranged from 94-96%, with the lowest value exhibited by the sample N, and the highest by the sample H. Sample H displayed also the lowest amount of superficial ASX, 0.026%, and sample N the highest, 0.0325%. The sASX was in general higher in comparison with SPI, but still very low (Table 7).

TABLE 7
Effects of the pretreatments on encapsulation efficiency
(EE %) and superficial ASX % of ASX PPI NPs

| ASX PPI NPs | EE % | sASX % |
|---|---|---|
| Non-treated | 94 ± 2.6 | 0.033 ± 0.0005 |
| Heat | 96 ± 0.5 | 0.027 ± 0.0001 |
| pH | 93 ± 1.4 | 0.028 ± 0.0012 |
| pH ± heat | 95 ± 0.3 | 0.027 ± 0.0003 |

### Rice Protein Isolate (RPI)

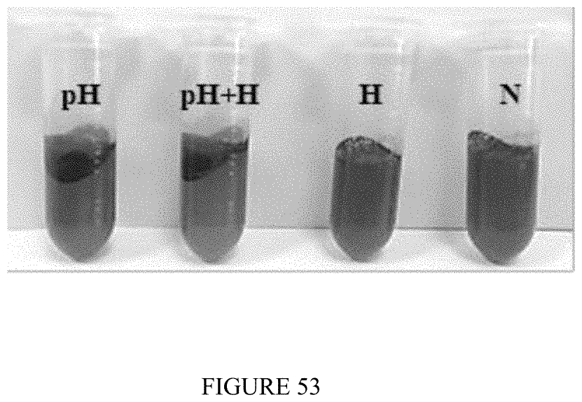

With the goal of identifying novel non-allergenic and gluten free protein candidates the inventors tried to employ also rice protein isolate (RPI). As shown in FIG. 53, it was impossible to obtain ASX NPs with the use of this protein as emulsifier: large amounts of non-encapsulated ASX and opalescence were observed for all the samples, probably because of the presence of aggregated or non-solubilized proteins. To the inventors knowledge no data are reported in literature involving this type of matrix for nanoparticle production by emulsification-solvent evaporation technique. In the study, the absence of a purification step aiming at removing the non-soluble protein fraction, i.e. glutelins, could have negatively affected the process. This protein fraction is characterized by high presence of disulfide bond and high molecular weight. These characteristics make RPI a scarce emulsifier and thus not suitable for NPs production.

The obtained data suggest that SPI and PPI could be used for the production on NPs with satisfactory results in terms of small particle size, narrow PDI and stability to coalescence. In both cases, the pre-treatment of proteins by heating and/or adjusting the pH did not lead to a significance variation of the encapsulation performance. For this reason, in the following experiment the inventors decided to avoid these treatments.

### Evaluation of ASX Release by Simulated Digestion

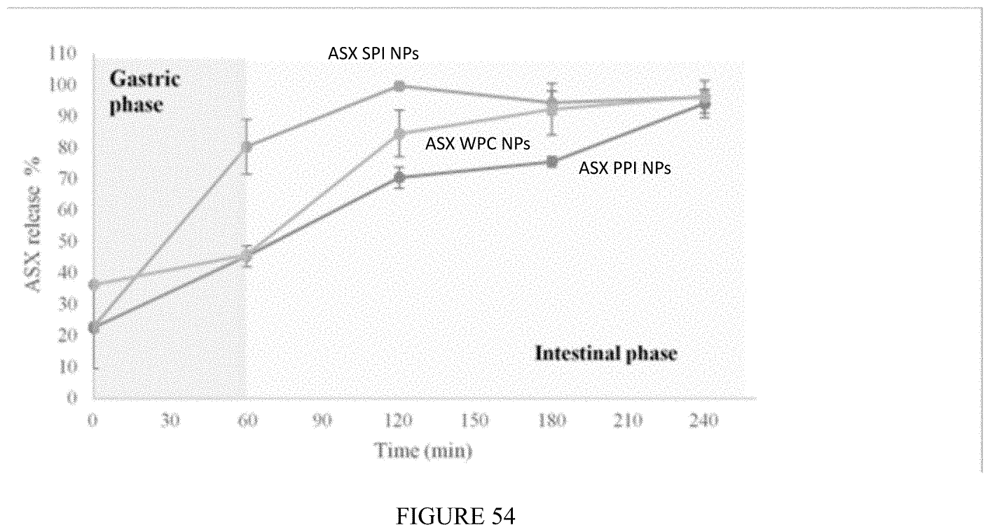

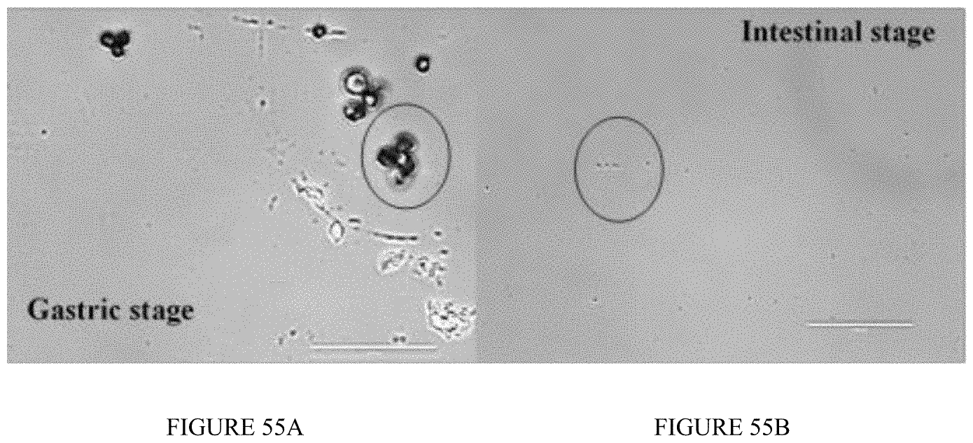

In vitro release of ASX from ASX SPI NPs and ASX PPI NPs, was assessed by simulated gastrointestinal digestion and the results were compared to ASX WPC NPs. These experimental data are important to study possible differences in the release profiles and thus in the bioaccessibility of encapsulated active molecule. Ideally, the release of ASX should occur in the intestine where absorption is supposed to take place. The time-dependent release of ASX is shown in FIG. 54. At time 0 ASX release from ASX WPC NPs was the highest, accounting for 36% of the total amount. ASX SPI and PPI NPs showed similar patterns, with 22-23% release. This initial release for all the formulations could be due to the acidic conditions of the gastric medium (pH 3) that could destabilize the NPs structure. To further confirm this hypothesis, the average size of ASX PPI NPs was analyzed by microscopy and DLS (FIGS. 55A-B), showing the formation of aggregates in the GS. It was impossible to obtain information about the particles size due to the high PDI (>1) and presence of aggregates in the sample. At the end of the GS the release of ASX from ASX WPC and PPI NPs was comparable, i.e. 45% and 46% respectively. Much higher was the release from ASX SPI NPs, that accounted for 80% of total ASX. For both ASX WPC and PPI NPs during the intestinal stage (IS) the release of ASX increased constantly within the first 3 hours except for the SPI formulation that, due to the large amount of ASX released during GS, showed a plateau after the first hour of IS reaching 99.7% release. At the end of the IS the amount of ASX released from the three formulation was equivalent ranging from 96 and 94%.

### Example 19

### Encapsulation of Sunflower Oil

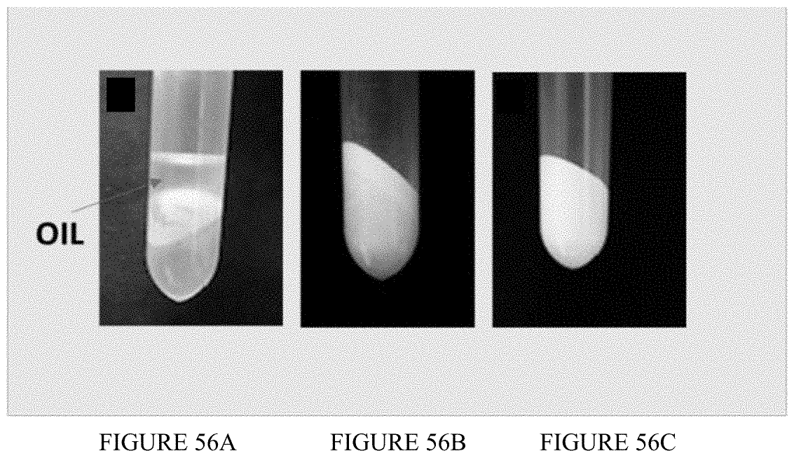

Sunflower oil was used for encapsulation, following the same procedure described previously for ASX NPs. In this case, 71% (w/w) of sunflower oil was successfully encapsulated. FIGS. 56A-C show the NPs solution obtained after the evaporation of the solvent, after centrifugation at 15000 rpm for 5 minutes. In the image is possible to observe the absence of non-encapsulated oil in FIG. 56B and FIG. 56C.

### Example 20

### Control of NPs Size

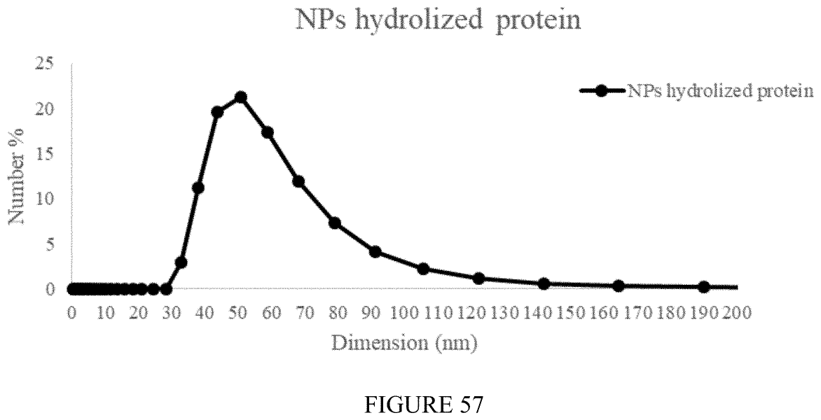

The dimension of the particles can be decreased and increased in different ways: modulating the amount of energy, type of equipment or the number of cycles used during the preparation of the nanoemulsion. For example, the inventors were able to decrease the NPs size from 100 nm to 60 nm sonicating the solution for 10 minutes instead of 5 minutes using, as already described, a potency of 10 W. On the contrary, the inventors were able to increase the size of the particles in the range of 1 μm, using a high shear homogenizer for 5 minutes at 13.500 rpm in order to produce an emulsion. It was also observed that, the concentration of solid inside the solution can have an impact on the size of the particles produced. The use of hydrolyzed protein, due to the low molecular weight can help in decrease the diameter of the particles. In this particular case, using the same formulation and preparation procedure previously described for ASX NPs, but using hydrolyzed whey protein instead of whey protein isolate, the inventors were able to obtain particles of 56 nm (FIG. 57).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1.-38. (canceled)

39. A composition comprising a plurality of particles, each particle having a diameter of 50 nm to 1000 nm, and comprising (i) at least one compound having a protein-based shell at least partially surrounding the at least one compound, and (ii) a coating comprising a polysaccharide encapsulating the at least one shelled compound.

40. The composition of claim 39, wherein a thickness of said coating is between 1 and 30 nm.

41. The composition of claim 39, wherein the concentration of said compound in said particle is 0.01 mg/g to 500 mg/g.

42. The composition of claim 39, wherein said at least one compound is soluble in an organic solvent.

43. The composition of claim 39, wherein said at least one compound is selected from the group consisting of a lipophilic compound, volatile organic compound, fragrance, protein, aroma, vitamin, lipophilic metabolite, partially lipophilic metabolite, or any combination thereof, astaxanthin, curcumin, omega 3, caffeine, beta-carotene, fish oil, sunflower oil, phytosterol, epigallocatechin gallate, Coenzyme Q10, cannabinoid or a functional derivative thereof, vitamin D, or any combination thereof.

44. The composition of claim 39, wherein said protein-based shell is selected from the group consisting of whey protein, soya protein, pea protein, fava bean protein, collagen or any combination thereof.

45. The composition of claim 39, further comprising a cationic polymer interacting with at least a portion of said protein-based shell.

46. The composition of claim 39, wherein said interaction is an electrostatic interaction.

47. The composition of claim 39, being selected from the group consisting of an edible composition, dietary supplement, pharmaceutical composition, agrochemical composition, or a cosmetic composition.

48. The composition of claim 39, being in the form of a powder.

49. The composition of claim 48, wherein said powder comprises 1% to 80% (w/w) of said at least one compound.

50. The composition of claim 48, wherein said powder has a content of 0.5 mg/g to 500 mg/g of said at least one compound.

51. The composition of claim 39, wherein at least 80% of said particles have a size in the rage of 5 nm to 300 nm when re-dispersed in water.

52. The composition of claim 39, having a polydispersity index of 0.05 to 0.5.

53. The composition of claim 39, wherein said partially surrounding is at least 85% of the total surface of said at least one compound.

54. The composition of claim 39, wherein said at least one compound has a zeta potential of −50 mV to −10 mV.

55. The composition of claim 39, wherein said plurality of particles have a zeta potential of 14 mV to 100 mV.

56. The composition of claim 39, wherein said composition is stable at a temperature between 25 and 40° C.

57. A method for producing the composition of claim 39, comprising the steps:
a. mixing a compound and a solvent, thereby obtaining a mixture;
b. mixing a protein to said mixture, thereby obtaining a nanoemulsion;
c. evaporating said solvent thereby obtaining a particle; and
d. drying said particle with a polysaccharide,
thereby encapsulating said compound.

58. The method of claim 57, wherein said solvent has a boiling point in the range of 35° C. to 80° C., optionally wherein said solvent comprises ethyl acetate.
